(19) United States
(12) Patent Application Publication
(10) Pub. No.: US 2018/0208920 A1
(43) Pub. Date: Jul. 26, 2018

(54) MOLECULAR MACHINES

(71) Applicant: Commonwealth Scientific and Industrial Research Organisation, Acton, Australian Capital Territory (AU)

(72) Inventors: Colin SCOTT, Acton, Australian Capital Territory (AU); Carol HARTLEY, Acton, Australian Capital Territory (AU); Charlotte WILLIAMS, Acton, Australian Capital Territory (AU); Quentin CHURCHES, Acton, Australian Capital Territory (AU); Judith SCOBLE, Acton, Australian Capital Territory (AU); Nicholas TURNER, Acton, Australian Capital Territory (AU); Nigel FRENCH, Acton, Australian Capital Territory (AU)

(21) Appl. No.: 15/746,299

(22) PCT Filed: Jul. 19, 2016
(86) PCT No.: PCT/AU2016/050641

§ 371 Date: Jan. 19, 2018

(30) Foreign Application Priority Data
Jul. 20, 2015 (AU) 2015902880
Jul. 24, 2015 (AU) 2015902961

(51) Int. Cl. C12N 11/18; C12N 11/06; C12P 9/00; C12N 9/12; C12N 9/04; C12N 9/02; C12N 9/18

(52) U.S. Cl. CPC C12N 11/18; C12N 11/06; C12P 9/00; C12N 9/1205; C12Y 207/0103; C12N 9/18; C12Y 101/01094; C12Y 106/03001; C12N 9/0036; C12Y 301/01; C12N 9/0006

(57) ABSTRACT

The present disclosure relates to isolated enzyme complexes comprising a tethered cofactor and at least two enzymes paired to catalyse an enzymatic reaction and recycle the cofactor.

### FIELD OF THE INVENTION

The present disclosure relates to isolated enzyme complexes comprising a tethered cofactor and at least two enzymes paired to catalyse an enzymatic reaction and recycle the cofactor.

### BACKGROUND OF THE INVENTION

Biocatalysts have the potential to significantly reduce the waste produced and energy cost in organic syntheses. In part, this is because the exquisite selectivity of biocatalysts (many of which operate at low temperatures and pressures) reduces the formation of unwanted side products, which has the additional benefit of simplifying downstream separation. Indeed, the number of organic syntheses in which enzymes are used as catalysts is increasing rapidly, due to their superior stereo- and regio-specificity under mild pH and temperature conditions (Leonida et al., 2001).

Various industrial processes are now performed by immobilising enzyme catalysts in flow reactors. Immobilizing enzyme catalysts in flow reactors has a number of advantages including enzyme reuse, enzyme stabilisation (in particular prevention of aggregation), continuous reaction processes and the prevention of contamination of product with enzyme.

Furthermore, coupling cascading enzyme reactions for the conversion of low value renewable feedstocks into high value products represents a keystone of renewable green chemistry.

However, one of the main limitations to the application of current enzyme systems to energy-intensive synthetic reactions is the cost of providing a continuous supply of diffusible cofactors or co-substrates (Zhao et al., 2003). Thus, there is an emerging requirement to develop improved enzyme catalysts, in particular for use in industrial processes and renewable green chemistry.

### SUMMARY OF THE INVENTION

The present inventors have found that stable enzyme fusions can be produced from various enzyme pairings. The present inventors have also found that various cofactors can be tethered to these fusions to form enzyme complexes capable of performing an enzymatic reaction and in situ cofactor regeneration.

Accordingly, in one aspect the present disclosure relates to an isolated enzyme complex comprising;

a) a cofactor,

b) a first enzyme that requires the cofactor to perform an enzymatic reaction, and

c) a second enzyme that recycles the cofactor,

wherein the first enzyme, second enzyme and cofactor form the enzyme complex through covalent attachments, and wherein the cofactor is covalently attached via a tether that allows the cofactor to be used by the first enzyme and recycled by the second enzyme.

In an example, the cofactor is selected from the group consisting of ATP/ADP, NAD+/NADH, NADP+/NADPH, and FAD+/FADH₂.

In an example, the cofactor has a ribonucleotide core. In an example, the tether is covalently attached to the ribonucleotide core via a C—N(carbon to nitrogen) bond to the base portion of the ribonucleotide core.

In an example, the tether comprises a polyethylene glycol (PEG) chain, hydrocarbon chain, a polypeptide, polynucleotide. In an example, the length of the polyethylene glycol chain is PEG₂-PEG₄₈ (i.e. (—CH₂CH₂O—)₂ to (—CH₂CH₂O—)₄₈. In an example, the length of the polyethylene glycol chain is PEG₂₄ (i.e. (—CH₂CH₂O—)₂₄). In an example, the length of the hydrocarbon chain is C₈-C₁₈. In an example, the length of the hydrocarbon chain is C₁₂-C₁₈. In an example, the length of the hydrocarbon chain is C₁₂.

In an example, the cofactor is tethered to one of the enzymes.

In an example, the first and second enzymes are covalently attached by a linker. In an example, the cofactor is tethered to the linker.

In an example, the linker is an amino acid linker. In an example, the linker comprises a Cys, a Thr, a Glu or a Lys amino acid residue. In an example, the linker comprises GlySerSer amino acid residue repeats (GlySerSer)ₙ. In an example, the linker comprises (GlySerSer)₃Cys(GlySerSer)₃.

The first enzyme can be any protein which is able to convert a suitable substrate into a product of interest. Examples of suitable first enzymes include, but are not limited to, a kinase, a dehydrogenase, an oxygenase, an aldolase, a reductase and a synthase.

The second enzyme can be any protein which is able to convert a cofactor of the first enzyme into a form in which it can be used by the first enzyme to convert the suitable substrate into the product of interest. Examples of suitable second enzymes include, but are not limited to, a kinase, a dehydrogenase, an oxidase, a reductase, and a peroxidase.

In an example, the enzyme complex further comprises a covalently attached conjugation module for conjugating the complex to a solid support. In an example, the conjugation module is covalently attached to the first enzyme or the second enzyme by a linker. In an example, the linker is a linker referenced in the above examples.

In an example, the conjugation module is a protein. Examples of proteins that can be used as part of the conjugation module include, but are not necessarily limited to, an esterase, streptavidin, glutathione S-transferase, a metal binding protein, a cellulose binding protein, a maltose binding protein and an antibody or antigen binding fragment thereof.

In an example, the enzyme complex is covalently or non-covalently attached to a solid support.

In an example, the solid support is a functionalised polymer. In an example, the functionalised polymer is selected from, but not necessarily limited to, the group consisting of: agarose, cotton, polyacrylonitrile, polyester, polyamide, protein, nucleic acids, polysaccharides, carbon fibre, graphene, glass, silica, polyurethane and polystyrene.

In an example, the solid support is in the form of a bead, a matrix, a woven fibre or a gel.

In another aspect, the present disclosure relates to a method for producing an enzyme complex of the invention, the method comprising:

i) expressing a polynucleotide encoding a chimeric protein comprising the first enzyme and the second enzyme in a host cell or cell-free expression system; and

ii) attaching the cofactor to the chimeric protein via the tether.

In an example, the first enzyme and the second enzyme are separated by a linker and step ii) comprises covalently attaching the tether to the linker.

In an example, the chimeric protein may further comprise an above exemplified conjugation module protein. In an example, the method further comprises conjugating the enzyme complex to a solid support.

The host cell may be any cell type. Examples include, but are not limited to, a bacterial cell, a yeast cell, a plant cell or an animal cell.

Enzyme complexes of the invention can be used in a wide variety industrial and non-industrial systems for producing a product of interest where the synthesis requires a recyclable cofactor. The ability of the enzyme complex of the invention to recycle the cofactor reduces the cost and work load associated with conducting these types of reactions. Thus, in a further aspect the present invention provides a method for producing a product, comprising,

i) providing an enzyme complex according to the present disclosure and a substrate of the first enzyme, and

ii) incubating the enzyme complex and substrate for a time and under conditions sufficient for the first enzyme to convert the substrate to the product and for the second enzyme to recycle the cofactor for use by the first enzyme.

The product may be suitable for commercial sale, or an intermediary product required for the synthesis of a desired end product.

In an example, the method may comprise two or more enzymatic steps and at least two of the enzymatic steps may be performed using two different enzyme complexes of the present disclosure.

In an example, the method is performed in a bioreactor. In an example, the bioreactor is a continuous flow bioreactor.

In an example, the present disclosure relates to a bioreactor comprising an enzyme complex of the present disclosure.

In a further aspect, the present invention provides a composition comprising at least one enzyme complex of the invention. Such a composition may comprise a suitable carrier and/or excipient. Such a composition may be suitable for being used in a method of the invention for producing a product.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Expression and purification of bi-enzymatic fusion proteins TkG1pK:MsAK; BiF1 (a) and EcG3PD::CaNOX; BiF2 (b).

FIG. 2. Combined batch reaction with BiF1 and BiF2: conversion of glycerol to DHAP.

FIG. 3. Effect of pH (a) and overall yield (b) of large scale combined bi-enzymatic batch reactions with BiF1 and BiF2. Reactions were conducted at room temperature in 1 mL total volume with 100 mM glycerol, 500 μM each of both ATP and NAD, 100 mM acetyl phosphate and 400 μg/mL (˜4 μM) each bi-enzymatic fusion protein.

FIG. 4. A. Scheme of multi-enzyme reactions to convert glycerol via DHAP to sugar and sugar analogues using three different aldehyde acceptors. B. Multi-enzyme batch reaction conversions of glycerol to glycerol-3-phopshate, DHAP and chiral sugars using BiF1, BiF2 and aldolases from S. carnosus I (ScFruA) and T. caldophilus (TcFruA).

FIG. 5. Optimization of pH for multi-enzyme batch reactions to convert glycerol to fructose-1,6-biphosphate. Error bars present standard error of the mean (SEM; n=3).

FIG. 6. The structures of adenosine triphosphate (ATP, left) and nicotinamide adenine dinucleotide (NAD⁺).

FIG. 7. Scheme depicting optimised overall route to prepare functionalised NAD (N⁶-(2-aminoethyl)-b-nicotinamide adenine dinucleotide, referred to herein as N⁶-2AE-NAD).

FIG. 8. Scheme depicting optimised overall route to prepare examples of functionally tethered NAD constructs.

FIG. 9. Scheme for the preparation of an NAD-tether group suitable for attaching to a linker. The scheme shows reaction of N⁶-2AE-NAD with an maleimide-PEG-NHS linker to produce an NAD-tether group terminating in a maleimide group.

FIG. 10. The BiF2 was purified by gel filtration on a S200 2660 column equilibrated with PBS containing 0.1 mM TCEP and the absorbance at 280 nm, 259 nm and 450 nm was monitored. The fractions pooled for conjugation are indicated with red arrows. Gel filtration standards (BioRad) were run on the column; the volume where each protein elutes are indicated below the chromatogram.

FIG. 11. The NAD-2AE-PEG₂₄-BiF2 conjugate was purified by gel filtration on a S200 2660 column equilibrated with PBS containing 0.1 mM TCEP and the absorbance at 280 nm, 259 nm and 450 nm was monitored.

FIG. 12. The UV-vis spectra of BiF2 and NAD-2AE-PEG₂₄-BiF2.

FIG. 13. The UV-vis spectra of denatured high MW and low MW fractions of BiF2 and NAD-2AE-PEG₂₄-BiF2.

FIG. 14. Aldolase coupled reactions demonstrate the production of DHAP by NAD-2AE-PEG₂₄-BiF2 fusion protein biocatalysts without the addition of exogenous cofactor.

FIG. 15. Converson of glycerol-3-phosphate into DHAP with concomitant recycling of tethered NAD (TriF2). Key: − is no added NAD; + added 1 mM NAD; unc—unconjugated; conj—conjugated to NAD-2AE-PEG₂₄.

FIG. 16. Comparative activity of two different variations of TriF1 with different spacer lengths between the bienzymatic fusion component and the esterase component of the trienzymatic fusion protein.

FIG. 17. Thermal stability of tri-enzymatic fusion protein (TkG1pK:MsAK::AaE2).

FIG. 18. Thermal stability (A) and storage stability (B) of tri-enzymatic fusion protein 2 (EcG3PD::CaNOX::AaE2).

FIG. 19. A. Scheme of multi-enzyme reactions to convert glycerol via DHAP to sugar and sugar analogues using three different aldehyde acceptors. B. Multi-enzyme batch reaction conversions of glycerol to glycerol-3-phopshate, DHAP and chiral sugars using TriF1 (with and without tethered ATP), TriF2 and aldolase enzyme from S. carnosus I (ScFruA). * Denotes that the value for DHAP in these cases is an estimate only, based on subtraction of known amount of added glyceraldehde-3-phosphate acceptor (which shares same molecular mass and m/z as DHAP).

FIG. 20. Gel filtration profile of the reaction to tether ATP-CM-C₆-PEG₂₄-MAL (ATP-carboxymethyl-hexyl-PEG₂₄-maleimide) to TriF1.

FIG. 21. Activity of tethered ATP-CM-C₆-PEG₂₄-TriF1 with and without added ATP. Reactions were performed in 0.5 mL reaction volume at pH 8.0 with 2 mM glycerol substrate, and 100 μM ATP was added where indicated.

FIG. 22. Optimization of tethering NAD-2AE-PEG₂₄-MAL cofactor to TriF2: activity with and without addition of 100 μM exogenous NAD+ illustrating efficient tethering of cofactor.

FIG. 23. Hierarchal, modular enzymatic flow reactor concept.

FIG. 24. Esterase activity of CaNOX::AaE2 and EcG3PD::CaNOX::AaE2 (TriF2) in the presence of TFK inhibitors.

FIG. 25. Comparative activity of NAD-tethered TriF2 immobilized by conjugation onto cotton cloth discs in the presence and absence of exogenous NAD+ comparative activity.

FIG. 26. Residence time distribution measured with 3 cm plug of cotton discs packed in the column measuring at 1 ml/min.

FIG. 27. Conversion yield of glycerol-3-phophate from TriF1Reactor2 as a function of flow rate.

FIG. 28. TriF1Reactor2 flow reactor stability: continuous production of glycerol-3-phosphate from glycerol at maximum yield rate for over 30 hours in the absence of exogenous ATP (top line; circles) and with 10 μM exogenous ATP (bottom line; squares).

FIG. 29. TriF2Reactor2 with and without added NAD cofactor.

FIG. 30. Immobilisation of TriF2 to Sepharose-trifluoroketone beads from purified protein or crude lysate.

FIG. 31. Triple nanomachine multi-enzyme reactor cascade to convert glycerol-3-phosphate and CBZ-aminopropanediol into the CBZ protected amino ketohexose phosphate. Percent substrate conversion with cumulative the CBZ protected amino ketohexose phosphate production (A and C) with rate of activity (B and D) for two different flow rates: 0.3 mL per minute (A and B) and 0.2 mL/min (C and D).

FIG. 32. Efficiency of triple nanomachine reactor multi-enzyme cascade to convert glycerol-3-phosphate and CBZ-aminopropanediol into the CBZ protected amino ketohexose phosphate. Average % conversion is shown for each reactor step.

FIG. 33. Coupling reaction between a divinyl-sulfone activated bead and the hexyl-TFK inhibitor, followed by covalent interaction of the TFK inhibitor-derivatised bead with a serine residue (Ser155) in the fusion enzyme esterase active site.

FIG. 34. Triple nanomachine multi-enzyme reactor cascade to convert glycerol-3-phosphate and CBZ-aminopropanediol into CBZ-amino ketohexose phosphate (or 1-(dihydrogen phosphate) 6-(N—CBZ)-amino-6-deoxy,-L-Sorbose). Percent substrate conversion with cumulative CBZ-amino ketohexose phosphate production (A and C) with rate of activity (B and D) for two different flow rates: 0.3 mL per minute (A and B) and 0.2 mL/min (C and D).

FIG. 35. Efficiency of triple nanomachine reactor multi-enzyme cascade to convert glycerol-3-phosphate and CBZ-aminopropanediol into CBZ-amino ketohexose phosphate. Average % conversion is shown for each reactor step.

FIG. 36. Serial nanomachine reactor design for the synthesis of D-fagomine, a commercially relevant aminocylitol anti-diabetic drug.

FIG. 37. Phosphotransfer reactor TriF1 R3: Conversion of glycerol and acetyl phosphate to G3P and acetate by immobilised ADP-2AE-PEG₂₄-TriF1 in a column (1.5 cm id, 12 cm) run at a flow rate of 0.25 mL/min.

FIG. 38. The oxidation reactor TriF2 R2: conversion of G3P to DHAP in a flow reactor. The immobilised NAD-2AE-PEG₂₄-TriF2 nanomachine beads prepared in the presence of 10 μM TCEP were packed into a column (1.5 cm id×16.5 cm). 10 mM G3P in 50 μM TCEP pH 8 was passed through the column at a flow rate of 0.25 mL/min and the amount of G3P remaining and DHAP produced determined by LCMS for fractions F1 to F10.

FIG. 39. Optimisation of immobilisation of BiF4 (ScFruA-AaE2) to Sepharose-DVS-hexyl-TFK beads in small scale batch reactions.

FIG. 40. The aldol condensation reactor ScFru-AaE2 R2: conversion of Cbz-aldehyde and DHAP into a chiral dihydroxyketonephopshate in a flow reactor. The immobilised ScFru-AaE2 nanomachine beads prepared in the presence of 10 μM TCEP were packed into a column (1.5 cm id×16.5 cm). 5 mM Cbz-aminopropanal and DHAP in 50 mM citrate buffer pH 7 was passed through the column at a flow rate of 0.1 mL/min and the amount of DHAP and Cbz-aminopropanal remaining quantified by LCMS for fractions F1 to F10. Whilst the expected Cbz-dihydroxyketophosphate product was detectable by LCMS from reactor fractions, it was not quantifiable due to a lack of available standard to establish a calibration curve.

FIG. 41. Nanofactory 1: Serial nanomachine reactors for the synthesis of the chiral (3S,4R) dihydroxyketophosphate precursor to anti-diabetic drug D-fagomine.

FIG. 42. Flux of substrates and products throughout operation of the nanofactory comprising serial phosphotransfer, oxidation and aldol condensation reactors for the synthesis of the chiral (3S,4R) dihydroxyketophosphate precursor to anti-diabetic drug D-fagomine. The reactors were fed 5 mM glycerol in 50 mM citrate buffer pH8.0 with 50 μM TCEP at 0.25 mL/min for 1200 minutes (20 hrs), and 60 fractions of 3 mL volume were collected for analysis.

### DETAILED DESCRIPTION OF THE INVENTION

### General Techniques

Unless specifically defined otherwise, all technical and scientific terms used herein shall be taken to have the same meaning as commonly understood by one of ordinary skill in the art (e.g., in cell culture, molecular genetics, enzymology, protein chemistry, biochemistry and bioprocessing).

Unless otherwise indicated, the recombinant protein, cell culture, chemical functionalisation and bioprocessing techniques utilised in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), J. E. Coligan et al., (editors) Current Protocols in Protein Science, John Wiley & Sons (including all updates until present) and G. T Hermanson, Bioconjugate Techniques, Third Edition Elsevier (2013).

### Enzyme Complex

As used herein, an “enzyme” is a protein that accelerates or catalyses chemical reactions. An enzyme may have one or more active sites that bind to a substrate or selection of substrates. An enzyme may be naturally occurring or it may be of synthetic origin.

The term “enzyme complex” is used in the context of the present disclosure to refer to the structure formed through covalent attachment of the first enzyme, the 20 second enzyme and the cofactor. The attachments may be direct, or indirect through an intervening moiety or moieties such as a linker. Various examples of covalent attachments are discussed below.

The terms “recycle”, “recycled” and “recycling” are used in the context of the present disclosure to define the capacity for conversion of a cofactor to a form that can be used by the first enzyme to catalyse an enzymatic reaction.

Various other components can be covalently attached to the “enzyme complex” of the present disclosure. For example, an additional enzyme can be covalently attached to the complex. In an example, a third, a fourth, a fifth, a sixth, a seventh, an eighth, a ninth or a tenth enzyme can be covalently attached to the complex. The additional enzyme(s) may catalyse a similar or different enzymatic reaction to the first or second enzymes of the complex. In another example, a conjugation module is covalently attached to the complex.

### First and Second Enzymes

The “first enzyme” can be any enzyme that uses a cofactor to catalyse an enzymatic reaction and the “second enzyme” can be any enzyme that recycles the cofactor. The selection of “first enzyme” is not particularly limited by enzyme type or activity. In various examples, the first enzyme may be an oxidoreductase (EC 1), a transferase (EC 2), a hydrolase (EC 3), a lyase (EC 4) or a isomerase (EC 5). In various examples the first enzyme has an activity selected from Table 1.

TABLE 1
Exemplary enzyme activity.

| | |
|---|---|
| Number | Activity |

Oxidoreductase (EC 1)

| | |
|---|---|
| EC 1.1 | Acting on the CH—OH group of donors |
| EC 1.1.1 | With NAD+ or NADP+ as acceptor |
| EC 1.1.2 | With a cytochrome as acceptor |
| EC 1.1.3 | With oxygen as acceptor |
| EC 1.1.4 | With a disulfide as acceptor |
| EC 1.1.5 | With a quinone or similar compound as acceptor |
| EC 1.1.9 | With a copper protein as acceptor |
| EC 1.1.98 | With other, known, physiological acceptors |
| EC 1.1.99 | With unknown physiological acceptors |
| EC 1.2 | Acting on the aldehyde or oxo group of donors |
| EC 1.2.1 | With NAD+ or NADP+ as acceptor |
| EC 1.2.2 | With a cytochrome as acceptor |
| EC 1.2.3 | With oxygen as acceptor |
| EC 1.2.4 | With a disulfide as acceptor |
| EC 1.2.5 | With a quinone or similar compound as acceptor |
| EC 1.2.7 | With an iron-sulfur protein acceptor |
| EC 1.2.98 | With other, known, physiological acceptors |
| EC 1.2.99 | With unknown physiological acceptors |
| EC 1.3 | Acting on the CH—CH group of donors |
| EC 1.3.1 | With NAD+ or NADP+ as acceptor |
| EC 1.3.2 | With a cytochrome as acceptor |
| EC 1.3.3 | With oxygen as acceptor |
| EC 1.3.4 | With oxygen as acceptor |
| EC 1.3.5 | With a quinone or related compound as acceptor |
| EC 1.3.7 | With an iron-sulfur protein as acceptor |
| EC 1.3.8 | With a flavin as acceptor |
| EC 1.3.98 | With other, known, physiological acceptors |
| EC 1.3.99 | With unknown physiological acceptors |
| EC 1.4 | Acting on the CH—NH2 group of donors |
| EC 1.4.1 | With NAD+ or NADP+ as acceptor |
| EC 1.4.2 | With a cytochrome as acceptor |
| EC 1.4.3 | With oxygen as acceptor |
| EC 1.4.4 | With a disulfide as acceptor |
| EC 1.4.5 | With a quinone or similar compound as acceptor |
| EC 1.4.7 | With an iron-sulfur protein as acceptor |
| EC 1.4.9 | With a copper protein as acceptor |
| EC 1.4.98 | With other, known, physiological acceptors |
| EC 1.4.99 | With unknown physiological acceptors |
| EC 1.5 | Acting on the CH—NH group of donors |
| EC 1.5.1 | With NAD+ or NADP+ as acceptor |
| EC 1.5.3 | With oxygen as acceptor |
| EC 1.5.4 | With a disulfide as acceptor |
| EC 1.5.5 | With a quinone or similar compound as acceptor |
| EC 1.5.8 | With a flavin as acceptor |
| EC 1.5.98 | With other, known, physiological acceptors |
| EC 1.5.99 | With unknown physiological acceptors |
| EC 1.6 | Acting on NADH or NADPH |
| EC 1.6.1 | With NAD+ or NADP+ as acceptor |
| EC 1.6.2 | With a heme protein as acceptor |
| EC 1.6.3 | With oxygen as acceptor |
| EC 1.6.4 | With a disulfide as acceptor |
| EC 1.6.5 | With a quinone or similar compound as acceptor |
| EC 1.6.6 | With a nitrogenous group as acceptor |
| EC 1.6.7 | With an iron-sulfur protein as acceptor |
| EC 1.6.8 | With a flavin as acceptor |
| EC 1.6.99 | With unknown physiological acceptors |
| EC 1.7 | Acting on other nitrogenous compounds as donors |
| EC 1.7.1 | With NAD+ or NADP+ as acceptor |
| EC 1.7.2 | With a cytochrome as acceptor |
| EC 1.7.3 | With oxygen as acceptor |
| EC 1.7.5 | With a quinone or similar compound as acceptor |
| EC 1.7.6 | With a nitrogenous group as acceptor |
| EC 1.7.7 | With an iron-sulfur protein as acceptor |
| EC 1.7.99 | With unknown physiological acceptors |
| EC 1.8 | Acting on a sulfur group of donors |
| EC 1.8.1 | With NAD+ or NADP+ as acceptor |
| EC 1.8.2 | With a cytochrome as acceptor |
| EC 1.8.3 | With oxygen as acceptor |
| EC 1.8.4 | With a disulfide as acceptor |
| EC 1.8.5 | With a quinone or similar compound as acceptor |
| EC 1.8.7 | With an iron-sulfur protein as acceptor |
| EC 1.8.98 | With other, known, physiological acceptors |
| EC 1.8.99 | With unknown physiological acceptors |
| EC 1.9 | Acting on a heme group of donors |
| EC 1.9.3 | With oxygen as acceptor |
| EC 1.9.6 | With a nitrogenous group as acceptor |
| EC 1.9.98 | With other, known, physiological acceptors |
| EC 1.9.99 | With unknown physiological acceptors |
| EC 1.10 | Acting on diphenols and related substances as donors |
| EC 1.10.1 | With NAD+ or NADP+ as acceptor |
| EC 1.10.2 | With a cytochrome as acceptor |
| EC 1.10.3 | With oxygen as acceptor |
| EC 1.10.5 | With a quinone or related compound as acceptor |
| EC 1.10.9 | With a copper protein as acceptor |
| EC 1.10.99 | With unknown physiological acceptors |
| EC 1.11 | Acting on a peroxide as acceptor |
| EC 1.11.1 | Peroxidases |
| EC 1.11.2 | With H2O2 as acceptor, one oxygen atom of which |
| | is incorporated into the product |
| EC 1.12 | Acting on hydrogen as donor |
| EC 1.12.1 | With NAD+ or NADP+ as acceptor |
| EC 1.12.2 | With a cytochrome as acceptor |
| EC 1.12.5 | With a quinone or similar compound as acceptor |
| EC 1.12.7 | With an iron-sulfur protein as acceptor |
| EC 1.12.98 | With other, known, physiological acceptors |
| EC 1.12.99 | With unknown physiological acceptors |
| EC 1.13 | Acting on single donors with incorporation of molecular |
| | oxygen (oxygenases) |
| EC 1.13.11 | With incorporation of two atoms of oxygen |
| EC 1.13.12 | With incorporation of one atom of oxygen (internal |
| | monooxygenases or internal mixed function oxidases) |
| EC 1.13.99 | Miscellaneous |
| EC 1.14 | Acting on paired donors, with incorporation or reduction |
| | of molecular oxygen |
| EC 1.14.11 | With 2-oxoglutarate as one donor, and incorporation |
| | of one atom each of oxygen into both donors |
| EC 1.14.12 | With NADH or NADPH as one donor, and incorporation |
| | of two atoms of oxygen into one donor |
| EC 1.14.13 | With NADH or NADPH as one donor, and incorporation |
| | of one atom of oxygen |
| EC 1.14.14 | With reduced flavin or flavoprotein as one donor, and |
| | incorporation of one atom of oxygen |
| EC 1.14.15 | With reduced iron-sulfur protein as one donor, and |
| | incorporation of one atom of oxygen |
| EC 1.14.16 | With reduced pteridine as one donor, and incorporation |
| | of one atom of oxygen |
| EC 1.14.17 | With reduced ascorbate as one donor, and incorporation |
| | of one atom of oxygen |
| EC 1.14.18 | With another compound as one donor, and incorporation |
| | of one atom of oxygen |
| EC 1.14.19 | With oxidation of a pair of donors resulting in the |
| | reduction of molecular oxygen to two molecules of water |
| EC 1.14.20 | With 2-oxoglutarate as one donor, and the other |
| | dehydrogenated |
| EC 1.14.21 | With NADH or NADPH as one donor, and the other |
| | dehydrogenated |
| EC 1.14.99 | Miscellaneous |
| EC 1.15 | Acting on superoxide as acceptor |
| EC 1.16 | Oxidizing metal ions |
| EC 1.16.1 | With NAD+ or NADP+ as acceptor |
| EC 1.16.3 | With oxygen as acceptor |
| EC 1.16.5 | With a quinone or similar compound as acceptor |
| EC 1.16.8 | With flavin as acceptor |
| EC 1.16.9 | With a copper protein as acceptor |
| EC 1.16.98 | With other, known, physiological acceptors |
| EC 1.17 | Acting on CH or CH2 groups |
| EC 1.17.1 | With NAD+ or NADP+ as acceptor |
| EC 1.17.2 | With a cytochrome as acceptor |
| EC 1.17.3 | With oxygen as acceptor |
| EC 1.17.4 | With a disulfide as acceptor |
| EC 1.17.5 | With a quinone or similar compound as acceptor |
| EC 1.17.7 | With an iron-sulfur protein as acceptor |
| EC 1.17.98 | With other, known, physiological acceptors |
| EC 1.17.99 | With unknown physiological acceptors |
| EC 1.18 | Acting on iron-sulfur proteins as donors |
| EC 1.18.1 | With NAD+ or NADP+ as acceptor |
| EC 1.18.3 | With H+ as acceptor (now EC 1.18.99) |
| EC 1.18.6 | With dinitrogen as acceptor |
| EC 1.18.96 | With other, known, physiological acceptors |
| EC 1.18.99 | With H+ as acceptor |
| EC 1.19 | Acting on reduced flavodoxin as donor |
| EC 1.19.6 | With dinitrogen as acceptor |
| EC 1.20 | Acting on phosphorus or arsenic in donors |
| EC 1.20.1 | With NAD(P)+ as acceptor |
| EC 1.20.2 | With a cytochrome as acceptor |
| EC 1.20.4 | With disulfide as acceptor |
| EC 1.20.9 | With a copper protein as acceptor |
| EC 1.20.98 | With other, known, physiological acceptors |
| EC 1.20.99 | With unknown physiological acceptors |
| EC 1.21 | Acting on the reaction X-H + Y-H = X-Y |
| EC 1.21.3 | With oxygen as acceptor |
| EC 1.21.4 | With a disulfide as acceptor |
| EC 1.21.98 | With other, known, physiological acceptors |
| EC 1.21.99 | With unknown physiological acceptors |
| EC 1.22 | Acting on halogen in donors |
| EC 1.22.1 | With NAD+ or NADP+ as acceptor |
| EC 1.23 | Reducing C—O—C group as acceptor |
| EC 1.23.1 | With NADH or NADPH as donor |
| EC 1.23.5 | With a quinone or related compound as acceptor |
| EC 1.97 | Other oxidoreductases |

Transferase (EC 2)

| | |
|---|---|
| EC 2.1 | Transferring one-carbon groups |
| EC 2.1.1 | Methyltransferases |
| EC 2.1.2 | Hydroxymethyl-, Formyl- and Related Transferases |
| EC 2.1.3 | Carboxy- and Carbamoyltransferases |
| EC 2.1.4 | Amidinotransferases |
| EC 2.2 | Transferring aldehyde or ketonic groups |
| EC 2.2.1 | Transketolases and Transaldolases |
| EC 2.3 | Acyltransferases |
| EC 2.3.1 | Transferring groups other than amino-acyl groups |
| EC 2.3.2 | Aminoacyltransferases |
| EC 2.3.3 | Acyl groups converted into alkyl on transfer |
| EC 2.4 | Glycosyltransferases |
| EC 2.4.1 | Hexosyltransferases |
| EC 2.4.2 | Pentosyltransferases |
| EC 2.4.99 | Transferring other glycosyl groups |
| EC 2.5 | Transferring alkyl or aryl groups, other than methyl groups |
| EC 2.5.1 | Transferring Alkyl or Aryl Groups, Other than Methyl |
| | Groups |
| EC 2.6 | Transferring nitrogenous groups |
| EC 2.6.1 | Transaminases |
| EC 2.6.2 | Amidinotransferases |
| EC 2.6.3 | Oximinotransferases |
| EC 2.6.99 | Transferring Other Nitrogenous Groups |
| EC 2.7 | Transferring phosphorus-containing groups |
| EC 2.7.1 | Phosphotransferases with an alcohol group as acceptor |
| EC 2.7.2 | Phosphotransferases with a carboxy group as acceptor |
| EC 2.7.3 | Phosphotransferases with a nitrogenous group as acceptor |
| EC 2.7.4 | Phosphotransferases with a phosphate group as acceptor |
| EC 2.7.5 | Phosphotransferases with regeneration of donors, apparently |
| | catalysing intramolecular transfers |
| EC 2.7.6 | Diphosphotransferases |
| EC 2.7.7 | Nucleotidyltransferases |
| EC 2.7.8 | Transferases for other substituted phosphate groups |
| EC 2.7.9 | Phosphotransferases with paired acceptors |
| EC 2.7.10 | Protein-tyrosine kinases |
| EC 2.7.11 | Protein-serine/threonine kinases |
| EC 2.7.12 | Dual-specificity kinases (those acting on Ser/Thr and Tyr |
| | residues) |
| EC 2.7.13 | Protein-histidine kinases |
| EC 2.7.14 | Protein-histidine kinases |
| EC 2.7.99 | Other protein kinases |
| EC 2.8 | Transferring sulfur-containing groups |
| EC 2.8.1 | Sulfurtransferases |
| EC 2.8.2 | Sulfotransferases |
| EC 2.8.3 | CoA-transferases |
| EC 2.8.4 | Transferring alkylthio groups |
| EC 2.9 | Transferring selenium-containing groups |
| EC 2.9.1 | Selenotransferases |
| EC 2.10 | Transferring molybdenum- or tungsten-containing groups |
| EC 2.10.1 | Molybdenumtransferases or tungstentransferases with |
| | sulfide groups as acceptors |

Hydrolase (EC 3)

| | |
|---|---|
| EC 3.1 | Acting on ester bonds |
| EC 3.1.1 | Carboxylic ester hydrolases |
| EC 3.1.2 | Thioester hydrolases |
| EC 3.1.3 | Phosphoric monoester hydrolases |
| EC 3.1.4 | Phosphoric diester hydrolases |
| EC 3.1.5 | Triphosphoric monoester hydrolases |
| EC 3.1.6 | Sulfuric ester hydrolases |
| EC 3.1.7 | Diphosphoric monoester hydrolases |
| EC 3.1.8 | Phosphoric triester hydrolases |
| EC 3.1.11 | Exodeoxyribonucleases producing 5′-phosphomonoesters |
| EC 3.1.12 | Exodeoxyribonucleases producing 3′-phosphomonoesters |
| EC 3.1.13 | Exoribonucleases producing 5′-phosphomonoesters |
| EC 3.1.14 | Exoribonucleases producing 3′-phosphomonoesters |
| EC 3.1.15 | Exonucleases active with either ribo- or deoxyribonucleic |
| | acids and producing 5′-phosphomonoesters |
| EC 3.1.16 | Exonucleases active with either ribo- or deoxyribonucleic |
| | acids and producing 3′-phosphomonoesters |
| EC 3.1.21 | Endodeoxyribonucleases producing 5′-phosphomonoesters |
| EC 3.1.22 | Endodeoxyribonucleases producing 3′-phosphomonoesters |
| EC 3.1.25 | Site-specific endodeoxyribonucleases specific for altered |
| | bases |
| EC 3.1.26 | Endoribonucleases producing 5′-phosphomonoesters |
| EC 3.1.27 | Endoribonucleases producing 3′-phosphomonoesters |
| EC 3.1.30 | Endoribonucleases active with either ribo- or |
| | deoxyribonucleic acids and producing 5′- |
| | phosphomonoesters |
| EC 3.1.31 | Endoribonucleases active with either ribo- or |
| | deoxyribonucleic acids and producing 3′- |
| | phosphomonoesters |
| EC 3.2 | Glycosylases |
| EC 3.2.1 | Glycosidases, i.e. enzymes hydrolysing O- and 5-glycosyl |
| | compounds |
| EC 3.2.2 | Hydrolysing N-glycosyl compounds |
| EC 3.2.3 | Hydrolysing S-Glycosyl compounds (discontinued) |
| EC 3.3 | Acting on ether bonds |
| EC 3.3.1 | Thioether and trialkylsulfonium hydrolases |
| EC 3.3.2 | Ether hydrolases |
| EC 3.4 | Acting on peptide bonds (Peptidases) |
| EC 3.4.11 | Aminopeptidases |
| EC 3.4.13 | Dipeptidases |
| EC 3.4.14 | Dipeptidyl-peptidases and tripeptidyl-peptidases |
| EC 3.4.15 | Peptidyl-dipeptidases |
| EC 3.4.16 | Serine-type carboxypeptidases |
| EC 3.4.17 | Metallocarboxypeptidases |
| EC 3.4.18 | Cysteine-type carboxypeptidases |
| EC 3.4.19 | Omega peptidases |
| EC 3.4.21 | Serine endopeptidases |
| EC 3.4.22 | Cysteine endopeptidases |
| EC 3.4.23 | Aspartic endopeptidases |
| EC 3.4.24 | Metalloendopeptidases |
| EC 3.4.25 | Threonine endopeptidases |
| EC 3.4.99 | Endopeptidases of unknown catalytic mechanism |
| EC 3.5 | Acting on carbon-nitrogen bonds, other than peptide bonds |
| EC 3.5.1 | In linear amides |
| EC 3.5.2 | In cyclic amides |
| EC 3.5.3 | In linear amidines |
| EC 3.5.4 | In cyclic amidines |
| EC 3.5.5 | In nitriles |
| EC 3.5.99 | In other compounds |
| EC 3.6 | Acting on acid anhydrides |
| EC 3.6.1 | In phosphorus-containing anhydrides |
| EC 3.6.2 | In sulfonyl-containing anhydrides |
| EC 3.6.3 | Acting on acid anhydrides; catalysing transmembrane |
| | movement of substances |
| EC 3.6.4 | Acting on acid anhydrides; involved in cellular and |
| | subcellular movement |
| EC 3.6.5 | Acting on GTP; involved in cellular and subcellular |
| | movement |
| EC 3.7 | Acting on carbon-carbon bonds |
| EC 3.7.1 | In ketonic substances |
| EC 3.8 | Acting on halide bonds |
| EC 3.8.1 | In C-halide compounds |
| EC 3.9 | Acting on phosphorus-nitrogen bonds |
| EC 3.10 | Acting on sulfur-nitrogen bonds |
| EC 3.11 | Acting on carbon-phosphorus bonds |
| EC 3.12 | Acting on sulfur-sulfur bonds |
| EC 3.13 | Acting on carbon-sulfur Bonds |

| | |
|---|---|
| Number | Name |

Lyase (EC 4)

| | |
|---|---|
| EC 4.1 | Carbon-Carbon Lyases |
| EC 4.1.1 | Carboxy-Lyases |
| EC 4.1.2 | Aldehyde-Lyases |
| EC 4.1.3 | Oxo-Acid-Lyases |
| EC 4.1.99 | Other Carbon-Carbon Lyases |
| EC 4.2 | Carbon-Oxygen Lyases |
| EC 4.2.1 | Hydro-Lyases |
| EC 4.2.2 | Acting on Polysaccharides |
| EC 4.2.3 | Acting on Phosphates |
| EC 4.2.99 | Other Carbon-Oxygen Lyases |
| EC 4.3 | Carbon-Nitrogen Lyases |
| EC 4.3.1 | Ammonia-Lyases |
| EC 4.3.2 | Lyases acting on Amides, Amidines, etc. |
| EC 4.3.3 | Amine-Lyases |
| EC 4.3.99 | Other Carbon-Nitrogen Lyases |
| EC 4.4 | Carbon-Sulfur Lyases |
| EC 4.5 | Carbon-Halide Lyases |
| EC 4.6 | Phosphorus-Oxygen Lyases |
| EC 4.7 | Carbon-Phosphorus Lyases |
| EC 4.99 | Other Lyases |

Isomerase (EC 5)

| | |
|---|---|
| EC 5.1 | Racemases and Epimerases |
| EC 5.1.1 | Acting on Amino Acids and Derivatives |
| EC 5.1.2 | Acting on Hydroxy Acids and Derivatives |
| EC 5.1.3 | Acting on Carbohydrates and Derivatives |
| EC 5.1.99 | Acting on Other Compounds |
| EC 5.2 | cis-trans-Isomerases |
| EC 5.3 | Intramolecular Oxidoreductases |
| EC 5.3.1 | Interconverting Aldoses and Ketoses |
| EC 5.3.2 | Interconverting Keto- and Enol-Groups |
| EC 5.3.3 | Transposing C═C Bonds |
| EC 5.3.4 | Transposing S—S Bonds |
| EC 5.3.99 | Other Intramolecular Oxidoreductases |
| EC 5.4 | Intramolecular Transferases |
| EC 5.4.1 | Transferring Acyl Groups |
| EC 5.4.2 | Phosphotransferases (Phosphomutases) |
| EC 5.4.3 | Transferring Amino Groups |
| EC 5.4.4 | Transferring Hydroxy Groups |
| EC 5.4.99 | Transferring Other Groups |
| EC 5.5 | Intramolecular Lyases |
| EC 5.99 | Other Isomerases |

Examples of suitable first enzymes include, but are not limited to, a kinase, a dehydrogenase, an oxygenase, an aldolase, a reductase and a synthase.

In an example, the kinase is selected from the group consisting of EC 2.7.1-EC 2.7.14. In another example, the kinase is selected from the group consisting of EC 2.7.1.1-EC 2.7.1.188.

In an example, the dehydrogenase is a NAD-dependent dehydrogenase. In an example, the dehydrogenase is a NADP-dependent dehydrogenase. In an example, the dehydrogenase is selected from the group consisting of EC 1.1.1.1-EC 1.1.1.386. In an example, the dehydrogenase is selected from the group consisting of EC 1.1.2.1-EC 1.1.2.8, EC 1.1.3.1-EC 1.1.3.47, EC 1.1.5.2-EC 1.1.5.10, EC 1.1.9.1, EC 1.1.98.1-EC 1.1.98.5, EC 1.1.99.1-EC 1.1.99.39, EC 1.2.1.1-EC 1.2.1.92, EC 1.3.1.1-EC 1.3.1.107, EC 1.20.1.1.

In an example, the oxygenase is a NAD-dependent oxygenase. In an example, the oxygenase is a NADP-dependent oxygenase. In an example, the oxygenase is selected from the group consisting of EC 1.14.12, EC 1.1.4.13, EC 1.14.21. In an example, the oxygenase is a monooxygenase. In an example, the monooxygenase is selected from the group consisting of EC 1.14.13.1-EC 1.14.13.203.

In an example, the aldolase is selected from the group consisting of EC 4.1.2.1 to EC 4.1.2.57.

In an example, the reductase is selected from the group consisting of EC 1.7.1.1-EC 1.7.1.15, EC 1.8.1.2-EC 1.8.1.19, EC 1.16.1.1-EC 1.16.1.10.

In an example, the synthase is selected from the group consisting of EC 1.14.21.1-EC 1.14.21.10.

In an example, the first enzyme is a glycerol kinase (EC 2.7.1.30) such as Thermococcus kodakarensis glycerol kinase (TkGlpk). In another example, the first enzyme is a glycerol-3-phosphate dehydrogenase (EC 1.1.1.8) such as Escherichia coli glycerol-3-phosphate dehydrogenase. In a further example, the first enzyme is an old yellow enzyme such as Shewanella yellow enzyme (SYE2) or Bacillus subtilis yellow enzyme (YqjM). In an example, the first enzyme is an alcohol dehydrogenase (EC 1.1.1.1) such as Geobacillus thermodenitrificans alcohol dehydrogenase.

In various examples, the second enzyme also has an activity selected from Table 1. However, the second enzyme is selected on the basis that it has the capacity to catalyse recycling of the cofactor used by the first enzyme. For instance, examples of suitable second enzymes include, but are not limited to, a kinase, a dehydrogenase, an oxidase, a reductase, and a peroxidase.

Where the first enzyme converts ATP to ADP to perform an enzymatic reaction, an appropriate second enzyme is an enzyme that has the capacity to catalyse recycling of ADP to ATP. For example, where the first enzyme is a glycerol kinase (EC 2.7.1.30), one of skill in the art would appreciate (at least from the EC number database record) that the first enzyme converts ATP to ADP to catalyse phosphorylation of glycerol and therefore, an appropriate second enzyme is an enzyme that has the capacity to recycle ATP from ADP such as a pyruvate kinase (EC 2.7.1.40).

Where the first enzyme converts NAD to NADH to catalyse an enzymatic reaction, an appropriate second enzyme is an enzyme that has the capacity to catalyse recycling of NADH to NAD. For example, where the first enzyme is glycerol-3-phosphate dehydrogenase (EC 1.1.1.8), one of skill in the art would appreciate that the first enzyme converts NAD to NADH to catalyse metabolism of glycerol-3-phosphate to DHAP and therefore an appropriate second enzyme is an enzyme that has the capacity to recycle NAD from NADH such as an NADH oxidase (EC 1.6.3.4).

Where the first enzyme converts NADPH to NADP to catalyse an enzymatic reaction, an appropriate second enzyme is an enzyme that has the capacity to catalyse recycling of NADP to NADPH. For example, where the first enzyme is a NADPH dehydrogenase (EC 1.6.99.1) such as Bacillus subtilis yellow enzyme, one of skill in the art would appreciate that the first enzyme converts NADPH to NADP to catalyse reduction of aldehydes/ketones and therefore an appropriate second enzyme is an enzyme that has the capacity to recycle NADPH from NADP such as a formate dehydrogensase (NADP) (EC 1.2.1.43).

In an example, the kinase is selected from the group consisting of EC 2.7.1.-EC 2.7.14. In an example, the kinase is selected from the group consisting of EC 2.7.4.1-EC 2.7.4.28, EC 2.7.6.1-EC 2.7.6.5. In an example, the kinase is an acetate kinase. In an example, the acetate kinase is selected from the group consisting of EC 2.7.2.12. In an example, the kinase is a pyruvate kinase. In an example, the pyruvate kinase is selected from the group consisting of EC 2.7.1.40.

In an example, the dehydrogenase is selected from the group consisting of EC 1.1.1.1-EC 1.1.1.386. In an example, the dehydrogenase is selected from the group consisting of EC 1.1.2.1-EC 1.1.2.8, EC 1.1.3.1-EC 1.1.3.47, EC 1.1.5.2-EC 1.1.5.10, EC 1.1.9.1, EC 1.1.98.1-EC 1.1.98.5, EC 1.1.99.1-EC 1.1.99.39, EC 1.2.1.1-EC 1.2.1.92, EC 1.3.1.1-EC 1.3.1.107, EC 1.8.1.2-EC 1.8.1.19, EC 1.12.1.2-EC 1.12.1.5. In an example, the dehydrogenase is an acyl CoA FAD dehydrogenase. In an example, the acyl CoA FAD dehydrogenase is selected from the group consisting of EC 1.3.8.1-EC 1.3.8.12.

In an example, the oxidase selected from the group consisting of EC 1.6.3. In an example, the oxidase is a NADH oxidase. In an example, the NADH oxidase is selected from the group consisting of EC 1.6.3.3, EC 1.6.3.4. In an example, the oxidase is a NADPH oxidase. In an example, the NADPH oxidase is selected from the group consisting of EC 1.6.3.1, EC 1.6.3.2.

In an example, the reductase is selected from the group consisting of EC 1.7.1.1-EC 1.7.1.15, EC 1.8.1.2-EC 1.8.1.19.

In an example, the peroxidase is a NADH peroxidase. In an example, the NADH peroxidase is selected from the group consisting of EC 1.11.1.1. In an example, the peroxidase is a NADPH peroxidase. In an example, the NADPH peroxidase is selected from the group consisting of EC 1.11.1.2.

In an example, the second enzyme is a pyruvate kinase (EC 2.7.1.40) such as Mycobacterium smegmatis ATP kinase (MsAK). In an example, the second enzyme is an NADH oxidase (EC 1.6.3.4) such as Clostridium aminoverlaricum NADH oxidase (CaNOX). In an example, the second enzyme an alcohol dehydrogenase (EC 1.1.1.1) such as Geobacillus thermodenitrificans alcohol dehydrogenase (GtADH). In another example, the second enzyme is a formate dehydrogenase (EC 1.2.1.43) such as C. boidinii formate dehydrogenase.

One of skill in the art will appreciate that the first and second enzymes of the complex may have broadly overlapping enzymatic functions. For example, the first enzyme may be an:

i) an oxidoreductase (EC 1);

ii) a transferase (EC 2);

iii) a hydrolase (EC 3);

iv) a lyase (EC 4); or,

v) an isomerase (EC 5).

and the second enzyme may also be:

i) an oxidoreductase (EC 1);

ii) a transferase (EC 2);

iii) a hydrolase (EC 3);

iv) a lyase (EC 4); or,

v) an isomerase (EC 5).

For example, both the first and second enzymes may be a kinase, a dehydrogenase or a reductase. Nonetheless, the first and second enzymes are distinguished according to their use of the cofactor tethered to the complex at least because the first enzyme uses the cofactor to perform an enzymatic reaction and the second enzyme recycles the cofactor.

One of skill in the art will be able to identify optimal enzymes for use in the enzyme complexes of the present disclosure via routine screening. In an example, an optimal first enzyme has the greatest enzymatic activity for performing the desired enzymatic reaction. In an example, an optimal second enzyme has the greatest enzymatic activity for cofactor recycling. Preferably, the first enzyme and second enzyme are matched so they have suitable activity under the same or similar conditions, such as temperature and pH.

For instance, various glycerol kinases can be screened to determine optimal first enzymes for performing an enzymatic reaction converting glycerol to glycerol-3-phosphate. In another example, various glycerol-3-phosphate dehydrogenases can be screened to determine optimal first enzymes for performing an enzymatic reaction converting glycerol-3-phosphate to dihydroxyacetone phosphate (DHAP). In another example, various alcohol dehydrogenases can be screened to determine optimal first enzymes for performing an enzymatic reaction converting 2-pentanone to (+)-2S,3R-pentanol. In another example, various enzymes can be screened to determine optimal second enzymes for recycling ATP from ADP. In this example, various ATP kinases could be screened. In another example, various enzymes can be screened to determine optimal second enzymes for recycling NAD from NADH. In this example, various NADH oxidases can be screened. In another example, various enzymes can be screened to determine optimal second enzymes for recycling NADP from NADPH. In this example, various formate dehydrogenases can be screened.

Optimal first and second enzymes can also be screened to determine optimal enzyme pairings for use in the enzyme complexes of the present disclosure. For example, enzyme complexes can be formed using optimal first and second enzymes and enzyme activity assessed. In an example, an optimal enzyme pairing provides the greatest enzymatic activity for performing the desired enzymatic reaction. In an example, an optimal enzyme pairing provides the greatest enzymatic activity for performing the desired enzymatic reaction and cofactor recycling.

In an example, enzymes forming the enzyme complexes of the present disclosure have substantially similar enzymatic activity when compared with their native state. In other examples, enzymes forming the enzyme complexes of the present disclosure may have reduced activity compared with their native state.

In an example, the first enzyme has at least about 99%, at least about 98%, at least about 97%, at least about 96%, at least about 95%, at least about 90%, at least about 85%, at least about 80%, at least about 75%, at least about 70%, at least about 60%, at least about 50%, at least about 40%, or at least about 30% activity compared to its native state.

In another example, the second enzyme has at least about 99%, at least about 98%, at least about 97%, at least about 96%, at least about 95%, at least about 90%, at least about 85%, at least about 80%, at least about 75%, at least about 70%, at least about 60%, at least about 50%, at least about 40%, or at least about 30% activity compared to its native state.

One of skill in the art can easily determine whether the attached enzymes that form the enzyme complexes of the present disclosure have substantially similar enzymatic activity when compared with their native state or whether their enzymatic activity is reduced. For example, attached enzymes can be compared with their unattached counterparts using various measures of enzymatic activity such as (K_{M}), K_{cat} (s⁻¹), K_{cat}/Kₘ. These measures can also be tracked over time at various time points separated by, for example, minutes, hours or days to monitor enzymatic activity.

In an example, enzyme activity of the first enzyme can be assessed in a reaction mixture comprising substrate and cofactor (e.g. ATP, NAD, NADP, FAD). Kinetics can be determined by varying the concentrations of substrate or cofactor whilst maintaining the other in excess. Enzyme activity of the second enzyme can be assessed in a reaction mixture comprising cofactor for recycling (e.g. ADP, NADH, NADPH, FADH₂) and a substrate. Kinetics can again be determined by varying the concentrations of cofactor for recycling or substrate whilst maintaining the other in excess. Cofactor use (e.g. ADP, NADH, NADPH, FADH₂ production from ATP, NAD, NADP, FAD) and recycling (e.g. ATP, NAD, NADP, FAD production from ADP, NADH, NADPH, FADH₂) can be determined using standard techniques such as via HPLC.

As an example, glycerol kinase (first enzyme) activity can be assessed in a reaction mixture comprising 1 mM glycerol, 10 mM MgCl₂, 50 mM NaHCO₃ buffer pH 9.0, 1 mM ATP with approximately 2 μg/mL enzyme (35 nM). Kinetics can be determined by varying the concentrations of ATP or glycerol whilst maintaining the other in excess, and kinetic determinants calculated using Hyper software (Easterby, J, Liverpool University). As an example, substrate and cofactor concentrations can be varied from 0.1 to 10×Kₘ.

Acetate kinase (second enzyme) activity can be assessed via a similar method that replaces ATP with ADP and glycerol with acetyl phosphate or phosphoenol pyruvate. Enzyme kinetics can then be determined by varying the concentrations of ADP or acetyl phosphate or phosphoenol pyruvate whilst maintaining the other in excess. ADP production from ATP and vice versa can be determined via HPLC.

The activity of other enzymes can be assessed using similar methods by providing the appropriate substrate and cofactor(s).

### Cofactor

The enzyme complex of the present disclosure comprises a tethered cofactor. The term “cofactor” is used in the context of the present disclosure to encompass compounds that are required for an enzyme to perform an enzymatic reaction. In an example, the cofactor is an organic cofactor. Examples of organic cofactors include, but are not limited to, co-enzymes, vitamins, vitamin derivatives, non-vitamins. Exemplary co-enzymes, vitamins, vitamin derivatives and non-vitamins are shown in the Table 2 below.

TABLE 2
Exemplary vitamin, vitamin derivative and non-vitamin cofactors
Cofactor

| Vitamins | Non-vitamins |
|---|---|
| Ascorbic acid | 3′-Phosphoadenosine-5′-phosphosulfate |
| Biotin | Adenosine triphosphate (ATP) |
| Cobalamine | Coenzyme B |
| Coenzyme A | Coenzyme M |
| Coenzyme F420 | Coenzyme Q |
| Flavin adenine | Cytidine triphosphate |
| dinucleotide (FAD) | |
| Flavin mononucleotide | Glutathione |
| Lipoamide | Heme |
| Menaquinone | Methanofuran |
| Methylcobalamin | Molybdopterin |
| NAD+ and NADP+ | Nucleotide sugars |
| Pyridoxal phosphate | Pyrroloquinoline quinone |
| Tetrahydrofolic acid | S-Adenosyl methionine |
| Thiamine pyrophosphate | Tetrahydrobiopterin |
| | Tetrahydromethanopterin |

In an example, the cofactor is a nicotinamide cofactor. In an example, the cofactor has a ribonucleotide core. For example, the cofactor can be selected from the group consisting of ATP/ADP, NAD+/NADH, NADP+/NADPH, acyl CoA/CoA and FAD+/FADH₂. In an example, the cofactor is ATP/ADP. In an example, the cofactor is NAD+/NADH. In an example, the cofactor is NADP+/NADPH. In an example, the cofactor is acyl CoA/CoA. In an example, the cofactor is FAD+/FADH₂.

In other examples, the cofactor is an inorganic cofactor such as a metal ion or iron-sulfur cluster. For example, the cofactor may be cupric, ferrous, ferric, magnesium, manganese, molybdenum, nickel or zinc.

One of skill in the art will appreciate that a suitable cofactor is dictated by the first enzyme in the complex. This is because the first enzyme of the complex requires the cofactor to perform an enzymatic reaction. For example, when the first enzyme is a kinase such as Thermococcus kodakarensis glycerol kinase, a suitable cofactor is ATP/ADP. In another example, when the first enzyme is a NAD-dependent dehydrogenase such as Escherichia coli glycerol-3-phosphate dehydrogenase or a NAD-dependent yellow enzyme such as Shewanella yellow enzyme, a suitable cofactor is NAD/NADH. In another example, when the first enzyme is a NADP-dependent dehydrogenase such as Geobacillus thermodenitrificans alcohol dehydrogenase or a NADP-dependent yellow enzyme such as Bacillus subtilis yellow enzyme, a suitable cofactor is NADP/NADPH. In another example, when the first enzyme is a Fructosyl amino acid oxidase (EC 1.5.3), a suitable cofactor is FAD/FADH₂.

In an example, the enzyme complex comprises:

i) Thermococcus kodakarensis glycerol kinase, Mycobacterium smegmatis ATP kinase, ATP/ADP;

ii) Escherichia coli glycerol-3-phosphate dehydrogenase, Clostridium aminoverlaricum NADH oxidase, NAD/NADH;

iii) Shewanella yellow enzyme, Geobacillus thermodenitrificans alcohol dehydrogenase, NAD/NADH;

iv) Geobacillus thermodenitrificans alcohol dehydrogenase, C. boidinii formate dehydrogenase, NADP/NADPH; or

v) Bacillus subtilis yellow enzyme, C. boidinii formate dehydrogenase, NADP/NADPH.

It will also be appreciated by those of skill in the art that particular enzymes may require more than one cofactor to perform an enzymatic reaction. However, the enzyme complex need not comprise each and every cofactor used by the first enzyme. In an example, the enzyme complex comprises one tethered cofactor. In this example, additional cofactors can be provided in a reaction medium for use by the first enzyme as required.

In an example, the enzyme complex comprises multiple tethered cofactors. For example, the enzyme complex can comprise at least two, at least three, at least four tethered cofactors.

### Cofactor Functionalisation

When present in the enzyme complex, the co-factor is covalently linked via a tether. In an example, cofactors are functionalised for attachment to a tether. In other words, the cofactor is reacted with a chemical moiety (or cofactor loading group) which facilitates attachment of the cofactor to a tether moiety. Methods of attaching a cofactor to a tether are well known in the art (see, for example, Buckman and Wray, 1992). In an example, the ribonucleotide core of a cofactor can be used as a site of functionalisation. For example, N⁶-substituted NAD, NADP or FAD can be produced by alkylation of NAD, NADP or FAD in the N(1)-position and then rearranging the alkylation product via Dimroth rearrangement using an aqueous medium. The resulting functionalised cofactors can then be either covalently bound to an enzyme complex or subject to enzymatic oxidation before covalent bonding. Exemplary alkylation agents include iodoacetic acid, propiolactone, 3,4-epoxy butyric acid or ethyleneimine. Variations on this method are disclosed in (Buckmann et al., 1989) and are also suitable for functionalising cofactors. For example, NAD or NADP can be alkylated with ethyleneimine to obtain the corresponding N(1)-(2-aminoethyl)-NAD or N(1)-(2-aminoethyl)-NADP and then rearranged in an aqueous medium to obtain the corresponding N⁶-(2-aminoethyl)-NAD or N⁶-(2-aminoethyl)-NADP. FAD can also be alkylated with ethyleneimine to obtain N(1)-(2-aminoethyl)-FAD and then rearranged in an aqueous medium to obtain the corresponding N⁶-(2-aminoethyl)-FAD.

Other cofactor loading groups are contemplated. For example, the functionalised cofactor may comprise a group of the formula —(CH₂)ₙNH₂ where n is an integer of from 2 to 20, comprise a group of the formula —C₂₋₆alkylene-O—(CH₂CH₂O)ₒ—C₂₋₆alkylene-NH₂ where o is an integer of from 1 to 10, or comprise a group of the formula —O—(CH₂CH₂O)ₚ—NH₂ where p is an integer of from 1 to 10. Such functionalised cofactors may for example be prepared by reaction of a suitable chloroheterocyclic-sugar-phosphate compound:

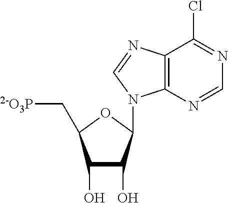

with an appropriate diamine compound, such as H₂N—O—(CH₂CH₂OCH₂CH₂OCH₂CH₂O—NH₂, or H₂N—(CH₂)₃—O—(CH₂CH₂O)₂—(CH₂)₃—NH₂, and reacting the resulting product with nicotinamide mononucleotide to produce the functionalised cofactor, see for example Cen et al, Org Biomol Chem, 2011, 9)4), p 987-993.

In an example, cofactors are functionalised via addition of a 6-AMX moiety. For example, 6-AMX-NAD⁺:

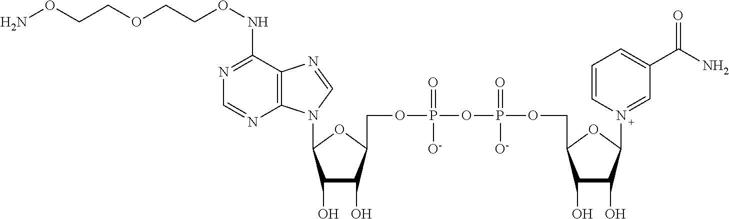

In another example, cofactors are functionalised via addition of 6-PEG-3 moiety. For example, 6-PEG3-NAD:

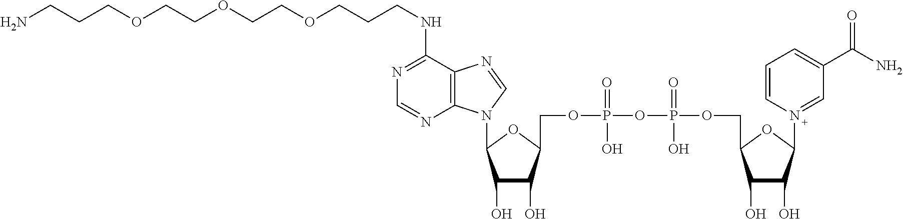

Other exemplary modifications to cofactors are shown in the Table 3 below.

TABLE 3
Exemplary modifications to cofactors.
N⁶-2AE-NAD

| | |
|---|---|
| (Willner et al., 2009) | N⁶-2AE-NAD, N⁶-(2-aminoethyl)-NAD⁺ 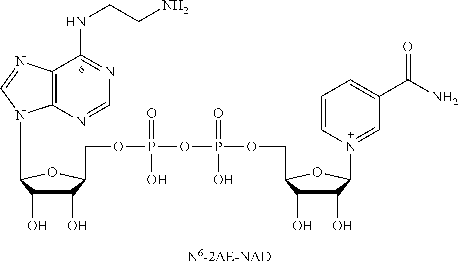 |
| (Willner et | N⁶-2AE-NAD |
| al., 2009; | |
| Bueckmann | |
| et al. 2002) | |

Other functional groups at N6

| | |
|---|---|
| (Sauve et al., 2011) | 6-AMX-NAD⁺ 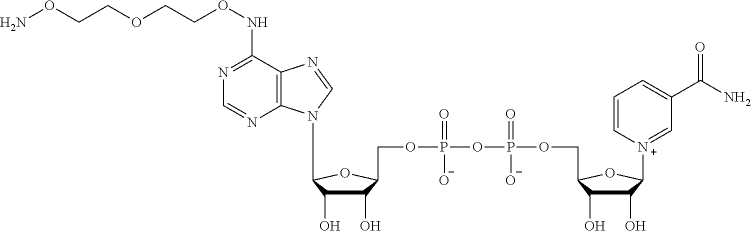 |
| (Bueckmann et al., 1996) | N6-(6-carboxyhexyl)-FAD 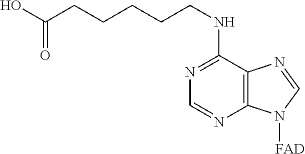 |
| (Mosbach et al., 1991) | N6-(N-(6-aminohexyl)-acetamide-NAD⁺ 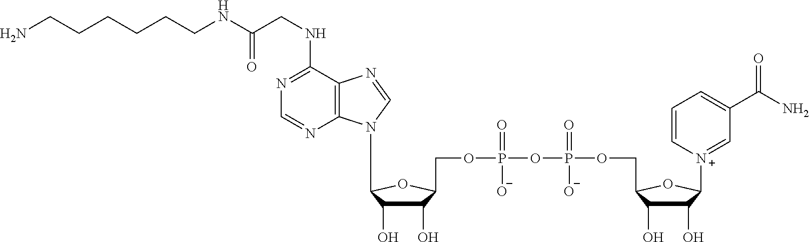 |

Reaction with epoxides

| | |
|---|---|
| (Wang et al., 2004) | Reaction with epoxide on a polymer that is attached to a glass surface. 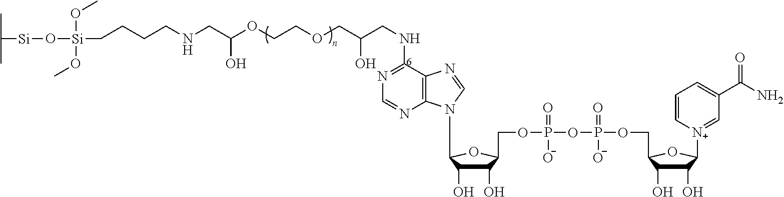 |
| (Bueckmann et al., 1993) | Reaction with epoxide. 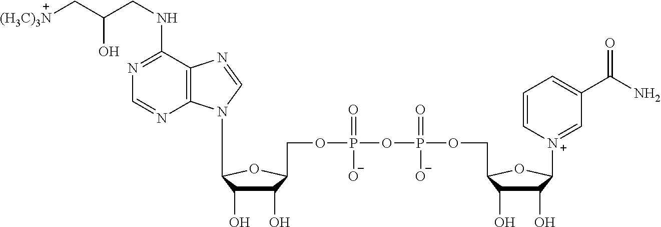 |
| (Fuller and Bright, 1980) | Reaction with epoxide at appended to a polymer backbone. 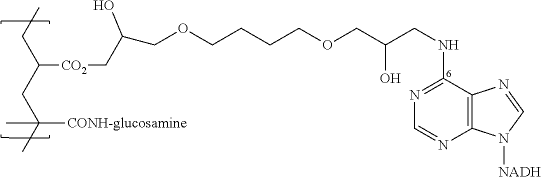 |

Other attachment site than N6

| | |
|---|---|
| (Willner et | Attachment of NAD through phenylboronic acid to sugar OH groups of NAD. |
| al., 2002) | |
| WO 2003/ | N⁶-2AE-NAD and N⁶-2AE-NADP |
| 100078 | |

Various cofactors such as NAD/NADH, NADP/NADPH and ATP/ADP can also be functionalised via halogenation of their adenine nucleus. Reaction of an adenine derivative halogenated at the 8-position with a suitable thiol compound bearing a further functional group such as a carboxylic group (e.g. nicotinamide-8-(2-carboxyethylthio) adenine dinucleotide), which can be coupled to various macromolecules; see, for example, U.S. Pat. No. 4,336,188.

In another example, a commercially available cofactor is tethered to the enzyme complex of the present disclosure. For example, N⁶-2AE-NAD is commercially available from Biolog Life Science Institute, Germany; Catalogue No.: N 013. CAS No.: [59587-50-7].

The chemical moiety (or cofactor loading group) with which the cofactor is reacted or functionalised may be any moiety which facilitates attachment of the cofactor to the tether and which does not destroy its biological activity. In one example, the functionalised cofactor comprises a pendant reactive group comprising an amino or carboxylic acid group, thereby facilitating attachment to tether moieties via routine chemistry steps. In one example the functionalised cofactor is N⁶-2AE-NAD.

When present in the final enzyme complex the cofactor loading group can be considered to form part of the tether. For example, where the functionalised cofactor is N⁶-2AE-NAD (e.g. produced by reaction of NAD with aziridine), the enzyme complex will comprise the group —CH₂CH₂—NH— resulting from reaction of the N⁶-2AE-NAD with a tether moiety.

In some cases, the enzyme complex is prepared by reacting a suitable cofactor-tether group bearing a reactive group capable of reacting with a complementary reactive group on an enzyme or on the linker. Such a cofactor-tether group may be prepared by reacting a functionalised cofactor (such as N⁶-2AE-NAD) with a tether moiety containing multiple orthogonal reactive groups. In those examples, a first reactive group on the tether moiety is capable of reacting with the functionalised cofactor, and a second reactive group on the tether moiety is capable or reacting with a reactive group on the enzyme or linker. In those cases, when present in the final enzyme complex, the tether can be understood as comprising the entire group extending between the cofactor and the attachment point on the enzyme or linker, including the residue of the cofactor loading group and including the residue of the tether moiety following synthesis of the enzyme complex.

Thus, functionalised co-factor intermediates can be tethered to constructs by reaction with, for example, SATA (N-succinimidyl S-acetylthioacetate) (e.g. SATA-PEG₄-NHS) or maleimide-PEG₂₄-NHS. Functionalised co-factor intermediates can also be tethered to constructs via a CO₂H group using peptide coupling agents, for example 8-nonenoic acid. PEGylated tethered constructs can be easily purified from unreacted co-factor using HPLC as they have significantly different retention times. In one example the tether moiety is maleimide-PEGₓ-NHS, i.e. a group of formula

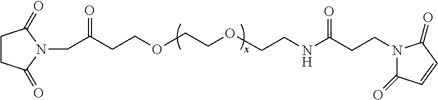

wherein x is an integer of from 4 to 24, e.g. 4, 6, 8, 12, 24.

Various other suitable tethers and examples of covalently attaching them to a cofactor and/or an enzyme complex are discussed below.

In some examples, the tether moiety comprises a central spacer group, and first and second reactive groups comprising different reactive functional groups. In one example the central spacer group is a hydrophobic group, for example a hydrocarbon group such as an alkylene group. In one example the central spacer group is an unbranched C₂₋₁₈, C₆₋₁₆, or C₈₋₁₄ alkylene group, for example an unbranched C₁₂ alkylene group. In one example the central spacer group is a hydrophilic spacer group, for example a PEG group (i.e. a group containing the subunit —CH₂CH₂O—. In one example the central spacer group is a PEG₂₋₄₈, PEG₂₋₂₄, PEG₂₋₁₂, or PEG₂₋₆ group (i.e. a group which is —(CH₂CH₂O)ₙ— wherein n is an integer in the range of from 2 to 48, from 2 to 24, from 2 to 12, or from 2 to 6. The nature of the reactive groups present in the first and second reactive moieties will depend on the nature of their respective reaction partners. For example, where the functionalised cofactor comprises a pendant reactive group comprising an amino group, it may be reacted with a tether moiety comprising a carboxylic acid group, for example in the presence of any amide coupling reagent such as uranium reagents (e.g. TSTU) or carbodiimide reagents (e.g. EDC).

Alternatively, it may be reacted directly with an activated ester group present in the tether moiety such as an NHS ester or pentafluorophenyl ester. In such cases, the resulting linkage is an amide linkage. As another example, where the functionalised cofactor comprises a pendant reactive group comprising a carboxylic acid group, it may be reacted with a tether moiety comprising an amine group, for example in the presence of an amide coupling agent. Alternatively, the functionalised cofactor may comprise an activated ester capable of reaction with an amino group present as part of the tether moiety. Again in those cases the resulting linkage is an amide linkage. As a further example, where reaction with a sulfhydryl group present on the enzyme or linker (e.g. a cysteine residue) is desired, one of the reactive groups present on the tether moiety may for example be a maleimide group.

The selected point of attachment for the components of the enzyme complex or additional components attached thereto unless otherwise stated is not particularly limited. However, in some examples, enzymes and other components such as cofactors and conjugation modules are attached at a “selected point of attachment”. The term, selected point of attachment is used herein to refer to a defined reactive point on the complex which allows for selective placement and attachment.

In one example, a tethered cofactor has a selected point of attachment on an enzyme of the enzyme complex. In another example, a tethered cofactor has a selected point of attachment on a covalent attachment connecting the first and second enzymes of an enzyme complex. In these examples, the cofactors selected point of attachment allows the cofactor to be used by the first enzyme and recycled by the second enzyme.

In an example, the selected point of attachment is a Cysteine, a Threonine, a Glutamine, a Glycine, a Serine or a Lysine amino acid residue. In another example, the selected point of attachment is a non-natural amino acid analogue to which a cofactor can be tethered. In another example, the selected point of attachment is a Cysteine, a Threonine, a Serine or a Lysine residue. Various methods are known in the art for selectively tethering a cofactor to a Cysteine, a Threonine, a Glutamine, a Glycine, a Serine or a Lysine amino acid residue. The most appropriate method will depend on the composition of the tether and the target amino acid residue. Exemplary attachment points for a tether residue include free sulfhydryl groups such as those of cysteine, free hydroxyl groups such as those of serine or threonine, the amine group of glycine or the amide group of glutamine.

In an example, the selected point of attachment for the tethered cofactor is a cysteine residue of the enzyme complex. In an example, the first and second enzymes are covalently attached via a linker comprising a cysteine residue and the selected point of attachment for the tethered cofactor is the cysteine residue of the linker. A tethered cofactor can be covalently attached to a cysteine residue using thiol reactive chemistries such as maleimide reaction chemistry. In short, a tethered cofactor is provided with a free maleimide group, for example as discussed above. Native disulphide bonds of the enzyme complex are then cleaved using a reducing agent such as tris(2-carboxyethyl)phosphine (TCEP) to produce free sulfhydryl groups that can crosslink (between pH 6.5 and 7.5) with free maleimide via thioether bonds. Various maleimide cross-linking kits are commercially available (e.g. ThermoFisherScientific).

In another example, a tethered cofactor can be selectively attached to a serine or threonine via O-linked glycosylation. In another example, a tethered cofactor can be selectively attached via a transglutaminase (EC 2.3.2.13) reaction wherein a transglutaminase enzyme catalyses the formation of an isopeptide bond between a free amine group (e.g., protein- or peptide-bound lysine) attached to the “linker” or “tether”, and the acyl group at the end of the side chain of protein- or peptide-bound glutamine. Other examples of chemical and/or enzymatic coupling of a tether to the enzyme complexes of the present disclosure are disclosed in, for example, WO/1987/005330, and Aplin and Wriston (1981).

### Covalent Attachment

The terms “linker” and “tether” are used in the context of the present disclosure to refer to covalent attachments between the components of the enzyme complex. In an example, an enzyme complex may comprise more than one linker. For example, an enzyme complex may have a first, a second, a third, a fourth or fifth linker for attaching various components. For example, an enzyme complex can comprise a first and second enzyme attached via a first linker and a conjugation module that is attached via a second linker. In an example, the enzyme complex may also comprise more than one tether. For example, an enzyme complex may have a first, a second, a third, a fourth or fifth tether for attaching multiple cofactors.

In an example, the first enzyme and second enzyme are covalently attached via a linker and the cofactor is covalently attached via a tether. In an example, a conjugation module is covalently attached to the enzyme complex via a linker.

A linker or tether can substantially be any biocompatible molecule that contains a functional group or a group that can be functionalised.

In an example, the length of the tether covalently attaching the cofactor to the complex allows the cofactor to be used by the first enzyme and recycled by the second enzyme. Any suitable tether which achieves the above function may be utilised. Examples of tethers include those comprising hydrocarbon chains (e.g. unbranched alkylene moieties), peptide chains, PEG-type or other polyether-type groups, and other polymeric groups (such as polyhydroxyacids). In one example, the tether consists of a chain of atoms linking the cofactor to the linker or enzyme, the chain consisting of from 40 to 500, from 40 to 400, from 40 to 300, from 40 to 200, from 40 to 100, from to 50, from 50 to 500, from 50 to 400, from 50 to 300, from 50 to 200, or from 50 to 100 atoms. For example, a tether of the formula:

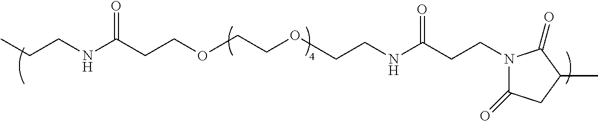

e.g. wherein the functionalised co-factor used is N⁶-2AE-NAD, the tether moiety used is maleimide-PEG₄-NHS, and the tether is attached to a linker via a cysteine side-chain sulfhydryl group, consists of 72 atoms linking the cofactor to the linker.

In an example, the linker or tether comprises hydrocarbons (e.g. the central spacer group may be an alkylene group), branched or unbranched, and said hydrocarbons being of chain length in the range of from C₂-C₂₅, C₂-C₂₀, C₂-C₁₅, C₂-C₁₀, C₂-C₉, C₂-C₈, C₂-C₇, C₂-C₆, C₂-C₅, C₂-C₄, or, at least C₂, at least C₃, at least C₄, at least C₅, at least C₆, at least C₇, at least C₈, at least C₉, at least C₁₀. In an example, the linker or tether comprises a branched or unbranched C₁₀-C₂₅, C₁₀-C₂₀, or C₁₀-C₁₅ hydrocarbon group. In an example, the linker or tether comprises a branched or unbranched C₁₅-C₅₀, C₁₅-C₂₅, or C₁₅-C₂₀ hydrocarbon group. In an example, the linker or tether comprises a branched or unbranched C₂₀-C₅₀, or C₂₀-C₂₅ hydrocarbon group. In an example, the linker or tether comprises a branched or unbranched C₂₅-C₅₀ hydrocarbon group. In one example, the linker or tether comprises an ether or polyether, (e.g. polyethylene oxide or polypropylene oxide), e.g. the central spacer group may be a PEG group as discussed above. In an example, the linker or tether may comprise an ether or polyether consisting of from 1-10, 1-5, 1-3 or at least 2 polyethylene oxide units or polypropylene oxide units.

In one example, the linker or tether is a polyalcohol, branched or unbranched such as polyglycol or polyethylene glycol (PEG) and derivatives thereof, such as for example O,O′-bis(2-aminopropyl)-polyethylene glycol 500 and 2,2′-(ethylene dioxide)-diethyl amine. For example, the linker or tether may comprise PEGₙ, wherein n is the number of PEG units. As referred to herein, and as indicated above, a PEG group is a group base on the subunit —(CH₂CH₂O)—, i.e. the term PEGₙ refers to a group of formula-(CH₂CH₂O)ₙ—

For example, the linker or tether may comprise PEGₙ having a chain length of PEG₂-PEG₅₀₀, PEG₂-PEG₄₀₀, PEG₂-PEG₃₀₀, PEG₂-PEG₂₀₀, PEG₂-PEG₁₀₀, PEG₂-PEG₅₀, PEG₂-PEG₂₅, PEG₂-PEG₂₀, PEG₂-PEG₁₅, PEG₂-PEG₁₀, PEG₂-PEG₉, PEG₂-PEG₈, PEG₂-PEG₇, PEG₂-PEG₆, PEG₂-PEG₅, PEG₂-PEG₄, or, at least PEG₂, at least PEG₈, at least PEG₄, at least PEG₅, at least PEG₆, at least PEG₇, at least PEG₈, at least PEG₉, at least PEG₁₀. In another example, the linker or tether is a polyurethane, polyhydroxy acid, polycarbonate, polyimide, polyamide, polyester, polysulfone comprising 1-500, 1-400, 1-300, 1-200, 1-100, 1-50, 1-25, 1-20, 1-15, 1-10, 1-9, 1-8, 1-7, 1-6, 1-5, 1-4, 1-3, or, at least 2 monomer units.

In another example, the linker or tether comprises an amino acid or a chain of amino acids or peptides. For example, the linker or tether may comprise a sequence of in the range of from 1-100, 1-75, 1-50, 1-25, or, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100 amino acid residues.

In an example, the linker or tether can comprise dipeptides, tripeptides, tetrapeptides, pentapeptides and so on.

In an example, the constituents of the amino acid linker or tether are L amino acids. For example, the linker or tether can comprise a Cys, a Thr, a Glu, a Gly, a Ser or a Lys amino acid residue.

In an example, the linker or tether comprises a Gly and a Ser. For example, the linker or tether can comprise GlySerSer or GlySerSer repeats (GlySerSerₙ). For example, the linker or tether can comprise GlySerSerₙ where n=1, n=2, n=3, n=4, n=5, n=6, n=7, n=8, n=9, n=10, n=11, n=12, n=13, n=14, n=15, n=16, n=17, n=18, n=19, n=20, n=21, n=22, n=23, n=24, n=25, n=26, n=27, n=28, n=29, n=30.

In another example, the linker or tether can comprise GlySerSerₙ-X-GlySerSerₙ, where n=1, n=2, n=3, n=4, n=5, n=6, n=7, n=8, n=9, n=10, n=11, n=12, n=13, n=14, n=15, n=16, n=17, n=18, n=19, n=20, n=21, n=22, n=23, n=24, n=25, n=26, n=27, n=28, n=29, n=30 and X is a Cys, a Thr, a Glu or a Lys.

In another example, the linker or tether can comprise GlySerSerₙ-XY-GlySerSerₙ, where n=1, n=2, n=3, n=4, n=5, n=6, n=7, n=8, n=9, n=10, n=11, n=12, n=13, n=14, n=15, n=16, n=17, n=18, n=19, n=20, n=21, n=22, n=23, n=24, n=25, n=26, n=27, n=28, n=29, n=30, X is a Cys, a Thr, a Glu or a Lys and Y=any amino acid.

In another example, the linker or tether can comprise GlySerSerₙ-X(Yₐ)-GlySerSerₙ, where n=1, n=2, n=3, n=4, n=5, n=6, n=7, n=8, n=9, n=10, n=11, n=12, n=13, n=14, n=15, n=16, n=17, n=18, n=19, n=20, n=21, n=22, n=23, n=24, n=25, n=26, n=27, n=28, n=29, n=30, X is a Cys, a Thr, a Glu or a Lys, Y=any amino acid or combination of amino acids and a=2, a=3, a=4, a=5, a=6, a=7, a=8, a=9, a=10, a=11, a=12, a=13, a=14, a=15, a=16, a=17, a=18, a=19, a=20, a=21, a=22, a=23, a=24, a=25, a=26, a=27, a=28, a=29, a=30.

In an example, the conjugation module is attached via a linker comprising GlySer or GlySer repeats (GlySerₙ). For example, the linker can comprise GlySerₙ where n=1, n=2, n=3, n=4, n=5, n=6, n=7, n=8, n=9, n=10, n=11, n=12, n=13, n=14, n=15, n=16, n=17, n=18, n=19, n=20, n=21, n=22, n=23, n=24, n=25, n=26, n=27, n=28, n=29, n=30.

In another example, the conjugation module is attached via a linker comprising GlySerₙ-Xₐ-GlySerₙ, where n=1, n=2, n=3, n=4, n=5, n=6, n=7, n=8, n=9, n=10, n=11, n=12, n=13, n=14, n=15, n=16, n=17, n=18, n=19, n=20, n=21, n=22, n=23, n=24, n=25, n=26, n=27, n=28, n=29, n=30, a=2, a=3, a=4, a=5, a=6, a=7, a=8, a=9, a=10, a=11, a=12, a=13, a=14, a=15, a=16, a=17, a=18, a=19, a=20, a=21, a=22, a=23, a=24, a=25, a=26, a=27, a=28, a=29, a=30 and X is any amino acid or combination of amino acids.

In other examples, the linker or tether can comprise amino acids selected from L-amino acids, D-amino acids or β-amino acids. For example, the linker or tether can comprise β-peptides.

In an example, the linker or tether can comprise molecules selected from the group consisting of thioxo-amino acids, hydroxy acids, mercapto acids, dicarbonic acids, diamines, dithioxocarbonic acids, acids and amines. In another example, the linker or tether comprises derivatised amino acid sequences or peptide nucleic acids (PNAs).

In another example, the linker or tether comprises one or more nucleic acids. For example, the nucleic acid linker or tether can have a length of 1-100, 1-75, 1-50, 1-25, or, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100 nucleic acid residues.

In an example, the linker or tether is a combination of the above referenced components.

In an example, the enzyme complex comprises a first enzyme and a second enzyme each covalently attached to a linker, and a cofactor covalently attached via a tether which is itself attached to the linker, wherein the linker comprises a sequence of amino acids, the tether comprises a tether moiety selected from the group consisting of a hydrocarbon chain (e.g. branched or unbranched alkylene moiety), a sequence of amino acids, or a PEG or other polyether group, and the cofactor is linked to the tether moiety via a cofactor loading group/co-factor functionalisation group.

Numerous methods for preparing the above referenced “linkers” and “tethers” and attaching them to a polypeptide, such as an enzyme, a compound or a cofactor are known in the art and are suitable for use in the present disclosure.

In an example, “linkers” and “tethers” are attached to a polypeptide using a suitable cross-linking functional group. Exemplary polypeptide functional groups include primary amines (—NH₂), carboxyls (—COOH), sulfhydryls (—SH), carbonyls (—CHO). Exemplary reagents for reacting an amine group with a carboxyl group include but are not limited to carbodiimide reagents (e.g. EDC, HOSu/DCC), phosphonium reagents (e.g., PyBOP, PyBrOP), uranium reagents (e.g., TSTU, COMU), imidazolium reagents (e.g., CDI), chloroformates via a mixed carbonic anhydride, acid chlorides by activation of the carboxylic acid with a chlorinating reagent. In some cases one of the reaction partners may contain an activated carboxylic group capable of reacting with an amine to form an amide, such as an NHS-ester, a pentafluorophenyl ester, a p-nitrophenyl ester, a hydroxymethyl phosphine group, or an imidoester.

Examples of suitable cross-linking functional groups capable of reacting with sulfhydryl groups include maleimide, haloacetyl (bromo- or iodo-), vinyl sulfone, pyridyldisulfide, thiosulfonate isocyanate and epoxide groups.

Examples of suitable cross-linking functional groups capable of reacting with an aldehyde group include amines, hydrazides and alkoxyamines. Other examples of reactive cross-linking groups include diazirines, aryl azides and isocyanates.

In another example, “linkers” and “tethers” can be functionalised and attached using various “click chemistry” strategies such as those disclosed in Kolb et al. (2001), WO 2003/101972, Malkoch et al. (2005), Li et al. (2009) and Gundersen et al. (2014).

In a further example, “linkers” and “tethers” can be attached via a transglutaminase reaction as discussed above.

### Conjugation

Enzyme complexes of the present disclosure can be conjugated to a solid support. An enzyme complex conjugated to a solid support can be covalently attached, non-covalently attached and/or immobilised to a support. A conjugated enzyme complex remains conformationally mobile relative to the support. The term “conformationally mobile” is used to refer to an enzyme complex that has a relatively fixed position on a support but is mobile in such a fixed position to be able to rotate about its fixed position to assume a conformation accessible to the tethered cofactor and a substrate or selection of substrates required to perform an enzymatic reaction.

In an example, the enzyme complex of the present disclosure can be conjugated to a support via a conjugation module. The term “conjugation module” is used in the context of the present disclosure to refer to a component that can react with a support or catalyse a reaction with a support to conjugate an enzyme complex to the support.

In an example, the conjugation module is a protein. For example, the conjugation module can be an esterase, streptavidin, biotin, a metal binding protein, a cellulose binding protein, a maltose binding protein, a polyhistidine, an antibody or antigen binding fragment thereof.

In an example, the conjugation module can be an enzyme. The conjugation module can be any enzyme that can form a covalent intermediate with an inhibitor (see for example, Huang et al., 2007). Suitable inhibitors will depend on the enzyme selected as the conjugation module and can be identified via routine screening. Various methods suitable for use in screening inhibitors are reviewed in (Williams and Morrison, 1979; Murphy, 2004). In an example, a suitable inhibitor will bind tightly to an enzyme conjugation module. Enzyme inhibitors that bind tightly are those inhibitors for which the binding constant, K₁, is at or below the concentration of the enzyme used in a screening assay [E]₀. The K₁ of tight binding inhibitors can be calculated using various methods. For example, K₁ of tight binding inhibitors can be calculated directly from the IC₅₀ value determined from graphical analysis of dose-response curves (Copeland, 1995).

In an example, the conjugation module can be a lipase, an esterase, glutathione S-transferase or serine-hydrolase.

In an example, the complex comprises:

i) Thermococcus kodakarensis glycerol kinase, Mycobacterium smegmatis ATP kinase, ATP/ADP; or

ii) Escherichia coli glycerol-3-phosphate dehydrogenase, Clostridium aminoverlaricum NADH oxidase, NAD/NADH; or;

iii) Shewanella yellow enzyme, Geobacillus thermodenitrificans alcohol dehydrogenase, NAD/NADH; or

iv) Geobacillus thermodenitrificans alcohol dehydrogenase, C. boidinii formate dehydrogenase, NADP/NADPH; or

v) Bacillus subtilis yellow enzyme, C. boidinii formate dehydrogenase, NADP/NADPH;

and a lipase, an esterase, glutathione S-transferase or serine-hydrolase. Accordingly, in this example, the conjugation module can be an esterase.

In an example, the conjugation module is an enzyme which enables conjugation to a support having a covalently attached trifluoroketone.

Various trifluoroketone containing molecules are known in the art. In an example, 1-hexanethiol is reacted with 1-bromo-3,3,3-trifluoroacetone to afford a hexyl trifluoroketone inhibitor.

In an example, the conjugation module is an esterase 2 from Alicyclobacillus acidophilus (see for example, Manco et al., 1998).

In an example, the complex comprises:

i) Thermococcus kodakarensis glycerol kinase, Mycobacterium smegmatis ATP kinase, ATP/ADP, Alicyclobacillus acidophilus esterase; or

ii) Escherichia coli glycerol-3-phosphate dehydrogenase, Clostridium aminoverlaricum NADH oxidase, NAD/NADH, Alicyclobacillus acidophilus esterase; or;

iii) Shewanella yellow enzyme, Geobacillus thermodenitrificans alcohol dehydrogenase, NAD/NADH; Alicyclobacillus acidophilus esterase; or

iv) Geobacillus thermodenitrificans alcohol dehydrogenase, C. boidinii formate dehydrogenase, NADP/NADPH; Alicyclobacillus acidophilus esterase; or

v) Bacillus subtilis yellow enzyme, C. boidinii formate dehydrogenase, NADP/NADPH; Alicyclobacillus acidophilus esterase.

In an example, the conjugation module is a non-protein. For example, a conjugation module can comprise various organic or inorganic molecules having a free reactive group. For example, the conjugation module can be a functional moiety or group on a linker or tether. In an example, the conjugation module is an enzyme inhibitor such as a trifluoroketone.

One of skill in the art will appreciate that the conjugation module will be selected based on the composition of the support. For example, a maltose binding protein will be selected as a conjugation module for conjugation of an enzyme complex to a support comprising maltose. In another example, a cellulose binding protein will be selected as a conjugation module for conjugation of an enzyme complex to a support comprising cellulose. In another example, an esterase will be selected as a conjugation module for conjugation of an enzyme complex to a support comprising an enzyme inhibitor such as a trifluoroketone. In another example, an enzyme inhibitor such as a trifluoroketone will be selected as a conjugation module for conjugation of an enzyme complex to a support comprising an esterase.

In an example, the conjugation module is covalently attached to the enzyme complex. In an example, the conjugation module is covalently attached to the first or second enzyme.

### Solid Supports

The enzyme complexes of the present disclosure can be conjugated to any functionalised or functionalisable materials that can be used as a support. Such materials can, for example, be present as support plates (monolithic blocks), membranes, films or laminates. In an example, the support is porous or non-porous.

In an example, the support comprises an inorganic or organic material. Exemplary, materials for a support include polyolefins, such as, for example, polyethylene, polypropylene, halogenated polyolefins (PVDF, PVC etc,), polytetrafluoroethylene and polyacrylonitrile. In other examples, materials for a support include ceramic, silicates, silicon and glass. In other examples, materials for a support include metallic materials such as gold or metal oxides, such as titanium oxide.

In an example the reactive surface on which the enzyme complex of the present disclosure is conjugated differs from the support material. For example, the material forming the (planar) reactive surface is present in the form of a film, which is then applied to a further base support material (e.g. for stabilisation).

In an example, the support comprises at least a first functionalisation site or group which is suitable to accomplish covalent bonding with the enzyme complex of the present disclosure. For example, the support can comprise reactive amino and/or carboxyl groups. For example, the support can comprise free primary hydroxyl groups. In an example, multiple successive functionalisation sites or groups can be provided on the support. In this example, multiple enzyme complexes can be attached to the support.

In another example, an enzyme complex of the present disclosure can be conjugated to a support via more than one functionalised site or group. In this example, the support comprises a first functionalised site or group and a further functionalised site or group such as a second, third, fourth, fifth, sixth, seventh, eighth, ninth or tenth functionalised site or group for attaching a single enzyme complex to a support.

In an example, the support is in the form of a membrane such as a mixed matrix membrane, a hollow fibre, a woven fibre, a particle bed, a fibre mat, beads or a gel. For example, the support can be in the form of agarose, agarose beads, cotton, carbon fibre, graphene or acrylamide.

The surface of a support can be functionalised via various methods in the art. The most appropriate method will depend on the supporting materials composition or at least the surface of the support. For example, cotton, agarose or other supports having primary hydroxyl groups available for chemical modification can be functionalised using commercially available cross-linking reagents such as a vinyl sulfone (VS), for example, divinyl sulfone (DVS). Alternatively, supports loaded with high density reactive groups are commercially available. Examples include DVS activated beads or agarose from suppliers such as Sigma-Aldrich. Other examples of functionalising supports with hydroxyl groups on the surface include reaction with biselectrophiles, such as for example, the direct carboxymethylation with bromoacetic acid; acylation with a corresponding amino acid derivative such as, for example, dimethylaminopyridine-catalysed carbodiimide coupling with fluorenyl methoxycarbonyl-3-aminopropionic acid or the generation of iso(thio-)cyanates by mono-conversion with corresponding bis-iso(thio)cyanates. In another example, starting from polyolefins as the material providing the supporting surface, a carboxyl group can be provided via oxidation with chromic acid or, for example, by high-pressure reaction with oxalyl chloride, plasma oxidation or radical or light-induced addition of acrylic acid.

Ceramics, glasses, silicon oxide and titanium oxide can be simply functionalised using substituted silanes available commercially, for example, aminopropyl triethoxy silane.

In an example, the enzyme complex can be non-covalently conjugated to a support. For example, the enzyme complex can be non-covalently conjugated by hydrophobically entrapping it so that the enzyme is stationary relative to a flowing aqueous substrate stream.

In this example, a suitable conjugated support comprises inert particulate material, for example, silica particles, each particle having multiple membranous elements. The enzyme being hydrophobic, preferentially locates itself between hydrophobic portions of the membrane elements, rather than migrating into the flowing aqueous stream.

An example of non-covalent conjugation applicable to an enzyme complex according to the present disclosure is described in U.S. Pat. Nos. 4,927,879 and 4,931,498. Other suitable support structures for non-covalent conjugation can be formed from silica, alumina, titania, or from resins having the necessary physical integrity.

### Producing an Enzyme Complex

The enzyme complexes of the present disclosure can comprise various “polypeptide” components including for example, enzymes, conjugation modules and various other polypeptide attachments such as linkers and tethers. In an example, the components of the enzyme complex can be produced or obtained from commercial suppliers separately and then covalently attached to form an enzyme complex.

Polypeptide components can be produced in a variety of ways, including production and recovery of natural polypeptides, production and recovery of recombinant polypeptides, and chemical synthesis of the polypeptides. In one example, an isolated polypeptide component (e.g. an enzyme) is produced by culturing a cell capable of expressing the polypeptide under conditions effective to produce the polypeptide, and recovering the polypeptide.

In another example, multiple components of the enzyme complex can be produced together. For example, enzyme complexes of the present disclosure can be produced by expressing a polynucleotide encoding a chimeric protein comprising the first enzyme and the second enzyme in a host cell or cell free expression system. A cofactor can then be attached to the chimeric protein via a tether. In another example, the expressed polynucleotide also encodes a linker separating the first enzyme and second enzyme. In this example, a cofactor can then be tethered to the linker. In another example, the expressed polynucleotide also encodes a conjugation module. The resulting enzyme complex can be attached to a solid support.

Various exemplary cells capable of expressing polypeptides, such as chimeric proteins, are discussed below. In one example, a capable cell has been transformed with a polynucleotide encoding a polypeptide component. As used herein, “transformed” or “transformation” is the acquisition of new genes in a cell by the incorporation of a polynucleotide.

The term “polynucleotide” is used interchangeably herein with the term “nucleic acid”. “Polynucleotide” refers to an oligonucleotide, nucleic acid molecule or any fragment thereof. It may be DNA or RNA of genomic or synthetic origin, double-stranded or single-stranded. Suitable polynucleotides may also encode secretory signals such as a signal peptide (i.e., signal segment nucleic acid sequences) to enable an expressed polypeptide to be secreted from the cell that produces the polypeptide. Examples of suitable signal segments include tissue plasminogen activator (t-PA), interferon, interleukin, growth hormone, viral envelope glycoprotein signal segments, Nicotiana nectarin signal peptide (U.S. Pat. No. 5,939,288), tobacco extensin signal, the soy oleosin oil body binding protein signal, Arabidopsis thaliana vacuolar basic chitinase signal peptide, as well as native signal sequences. In addition, the polynucleotide may encode intervening and/or untranslated sequences.

The terms “polypeptide” and “protein” are generally used interchangeably and refer to a single polypeptide chain which may or may not be modified by addition of non-amino acid groups or other component such as a tethered cofactor. The terms “proteins” and “polypeptides” as used herein also include variants, mutants, modifications, analogous and/or derivatives of the polypeptides of the disclosure as described herein. For example, the enzyme complex can comprise variants, mutants, modifications, analogous and/or derivatives of the enzymes encompassed by the present disclosure. In an example, these enzymes can have altered activity compared to their naturally occurring counterparts.

Mutant (altered) polypeptides can be prepared using any technique known in the art. For example, a polynucleotide encoding an enzyme encompassed by the present disclosure can be subjected to in vitro mutagenesis. Such in vitro mutagenesis techniques include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides of the disclosure are subjected to DNA shuffling techniques as broadly described by Harayama (1998). Products derived from mutated/altered DNA can readily be screened using techniques described herein to determine if they can be used in an enzyme complex of the present disclosure.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as important for function. Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 4 under the heading of “exemplary substitutions”.

TABLE 4
Exemplary substitutions

| Original | Exemplary |
|---|---|
| Residue | Substitutions |
| Ala (A) | val; leu; ile; gly; cys; ser; thr |
| Arg (R) | lys |
| Asn (N) | gln; his |
| Asp (D) | glu |
| Cys (C) | Ser; thr; ala; gly; val |
| Gln (Q) | asn; his |
| Glu (E) | asp |
| Gly (G) | pro; ala; ser; val; thr |
| His (H) | asn; gln |
| Ile (I) | leu; val; ala; met |
| Leu (L) | ile; val; met; ala; phe |
| Lys (K) | arg |
| Met (M) | leu; phe |
| Phe (F) | leu; val; ala |
| Pro (P) | gly |
| Ser (S) | thr; ala; gly; val; gln |
| Thr (T) | ser; gln; ala |
| Trp (W) | tyr |
| Tyr (Y) | trp; phe |
| Val (V) | ile; leu; met; phe; ala; ser; thr |

Polynucleotides can be expressed using a suitable recombinant expression vector. For example, a polynucleotide encoding the above referenced polypeptide components can be operatively linked to an expression vector. The phrase “operatively linked” refers to insertion of a polynucleotide molecule into an expression vector in a manner such that the molecule is able to be expressed when transformed into a host cell. Typically, the phrase refers to the functional relationship of a transcriptional regulatory element to a transcribed sequence. For example, a promoter is operably linked to a coding sequence, if it stimulates or modulates the transcription of the coding sequence in an appropriate host cell. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

As used herein, an expression vector is a DNA or RNA vector that is capable of transforming a host cell and of effecting expression of a specified polynucleotide molecule. Preferably, the expression vector is also capable of replicating within the host cell. Expression vectors can be either prokaryotic or eukaryotic, and are typically viruses or plasmids. Suitable expression vectors include any vectors that function (i.e., direct gene expression) in a recombinant cell, including in bacterial, fungal, endoparasite, arthropod, animal, and plant cells. Vectors of the disclosure can also be used to produce a polypeptide component(s) in a cell-free expression system, such systems are well known in the art.

Suitable vectors can contain heterologous polynucleotide sequences, that is polynucleotide sequences that are not naturally found adjacent to polynucleotide encoding the above referenced polypeptides. The vector can be either RNA or DNA, either prokaryotic or eukaryotic, and typically is a transposon (such as described in U.S. Pat. No. 5,792,294), a virus or a plasmid.

Suitable, expression vectors can also contain regulatory sequences such as transcription control sequences, translation control sequences, origins of replication, and other regulatory sequences that are compatible with the recombinant cell and that control the expression of specified polynucleotide molecules. Transcription control sequences are sequences which control the initiation, elongation, and termination of transcription. Particularly important transcription control sequences are those which control transcription initiation, such as promoter, enhancer, operator and repressor sequences. A variety of suitable transcription control sequences are known to those skilled in the art. Examples, include transcription control sequences which function in bacterial, yeast, arthropod, plant or mammalian cells, such as, but not limited to, tac, lac, trp, trc, oxy-pro, omp/lpp, rrnB, bacteriophage lambda, bacteriophage T7, T7lac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells.

A host cell suitable for preparing the components of the enzyme complex of the present disclosure includes a recombinant cell transformed with one or more polynucleotides that encode a component(s) of the enzyme complex, or progeny cells thereof. Transformation of a polynucleotide molecule into a cell can be accomplished by any method by which a polynucleotide molecule can be inserted into the cell. Transformation techniques include, but are not limited to, transfection, electroporation, microinjection, lipofection, adsorption, and protoplast fusion. Transformed polynucleotide molecules can remain extrachromosomal or can integrate into one or more sites within a chromosome of the transformed (i.e., recombinant) cell in such a manner that their ability to be expressed is retained.

Suitable host cells to transform include any cell that can be transformed with a polynucleotide encoding polypeptide component(s) of the enzyme complex. Suitable host cells can be endogenously (i.e., naturally) capable of producing polypeptide component(s) of the enzyme complex or can be capable of producing such polypeptides after being transformed with at least one polynucleotide molecule encoding the component(s). Suitable host cells include bacterial, fungal (including yeast), parasite, arthropod, animal and plant cells. Examples of host cells include Salmonella, Escherichia, Bacillus, Listeria, Saccharomyces, Spodoptera, Mycobacteria, Trichoplusia, BHK (baby hamster kidney) cells, MDCK cells, CRFK cells, CV-1 cells, COS (e.g., COS-7) cells, and Vero cells. Further examples of host cells are E. coli, including E. coli K-12 derivatives; Salmonella typhi; Salmonella typhimurium, including attenuated strains; Spodoptera frugiperda; Trichoplusia ni; and non-tumorigenic mouse myoblast G8 cells (e.g., ATCC CRL 1246). Suitable mammalian host cells include other kidney cell lines, other fibroblast cell lines (e.g., human, murine or chicken embryo fibroblast cell lines), myeloma cell lines, Chinese hamster ovary cells, mouse NIH/3T3 cells, LMTK cells and/or HeLa cells.

Recombinant techniques useful for increasing the expression of polynucleotide molecules of the present disclosure include, but are not limited to, operatively linking polynucleotide molecules to high-copy number plasmids, integration of the polynucleotide molecule into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotide molecules of the present disclosure to correspond to the codon usage of the host cell, and the deletion of sequences that destabilise transcripts.

Effective culture conditions include, but are not limited to, effective media, bioreactor, temperature, pH and oxygen conditions that permit polypeptide production. An effective medium refers to any medium in which a cell is cultured to produce a polypeptide of the present disclosure. Such medium typically comprises an aqueous medium having assimilable carbon, nitrogen and phosphate sources, and appropriate salts, minerals, metals and other nutrients, such as vitamins. Cells can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

### Uses

The enzyme complexes of the present disclosure can be used in any cofactor-dependant biocatalytic syntheses. Examples include enoane reduction, chiral amine synthesis and production of secondary alcohols, DHAP and pharmaceuticals such as Miglitol, precursors thereof such as the CBZ protected amino ketohexose phosphate or the anti-diabetic drug D-fagomine or the precursor thereof aminocyclitol.

In an example, an enzyme complex of the present disclosure is incubated with a substrate of the first enzyme for a time and under conditions sufficient for the first enzyme to convert the substrate to a product and the second enzyme to recycle the cofactor.

In one example, an enzyme complex comprising a kinase such as glycerol kinase and an ATP recycling enzyme such as ATP kinase with tethered ATP/ADP is used to catalyse conversion of glycerol into glycerol-3-phosphate. In another example, an enzyme complex comprising a NAD-dependent dehydrogenase such as glycerol-3-phopshate dehydrogenase and a NAD recycling enzyme such as NADH oxidase with tethered NAD/NADH is used to catalyse conversion of glycerol-3-phopshate into DHAP. In another example, an enzyme complex comprising an old yellow enzyme such as Shewanella yellow enzyme and a NAD recycling enzyme such as Geobacillus thermodenitrificans alcohol dehydrogenase with tethered NAD/NADH is used in enoate reduction, catalysing conversion of ketoisophorone into 6R-levodione. In another example, an enzyme complex comprising an NADP-dependent dehydrogenase such as Geobacillus thermodenitrificans alcohol dehydrogenase and a NADP recycling enzyme such as C. boidinii formate dehydrogenase with tethered NADP/NADPH is used to produce a chiral secondary alcohol, catalysing conversion of 2-pentanone into (+)-2S,3R-pentanol. In another example, an enzyme complex comprising an old yellow enzyme such as Bacillus subtilis yellow enzyme and a NAD recycling enzyme such as C. boidinii formate dehydrogenase with tethered NAD/NADH is used in chiral amine production, catalysing conversion of a 2-oxo acid (e.g. 2-oxo-methylvaleric acid into a D-BCAA (e.g. D-leucine).

In other examples, enzyme complexes of the present disclosures are combined to perform multiple reactions. For example, enzyme complexes can be used in a method comprising two or more enzymatic steps, wherein at least two of the enzymatic steps are performed using two different enzyme complexes of the present disclosure.

For example, a first enzyme complex comprising glycerol kinase and ATP kinase with tethered ATP/ADP is coupled with a further enzyme complex comprising glycerol-3-phopshate dehydrogenase and an NADH oxidase with tethered NAD/NADH. In this example, the first enzyme complex catalyses conversion of glycerol into glycerol-3-phosphate and the further enzyme complex catalyses conversion of glycerol-3-phopshate into DHAP.

In other examples, the enzyme complexes of the present disclosures are combined with other enzyme(s).

In various examples, the other enzyme is a galactose oxidase, such as galactose oxidase variant (GO_{M3-5}) and/or an aldolase such as Staphylococcus carnosus aldolase (ScFruA) or T. caldophilus aldolase, Escherichia coli Tagatose-biphosphate aldolase (EcTagA), Escherichia coli fuculose-1-phosphate aldolase (EcFucA) or Escherichia coli Rhamnulose-1-phosphate aldolase (EcRhuA).

For example, a first enzyme complex comprising glycerol kinase and ATP kinase with tethered ATP/ADP is coupled with a further enzyme complex comprising glycerol-3-phospshate dehydrogenase and an NADH oxidase with tethered NAD/NADH and an aldolase such as ScFruA, EcTagA, EcFucA or EcRhuA. In this example, the first enzyme complex catalyses conversion of glycerol into glycerol-3-phosphate, the further enzyme complex catalyses conversion of glycerol-3-phopshate into DHAP and the aldolase catalyses (via addition of an aldehyde) conversion of DHAP to various chiral sugars. In this example, DHAP can be reacted with for example glyceraldehyde-3-phosphate, propionaldehyde, acetylaldehyde or Cbz-aminopropanal.

In another example, a first enzyme complex comprising glycerol kinase and ATP kinase with tethered ATP/ADP is coupled with a further enzyme complex comprising glycerol-3-phospshate dehydrogenase and an NADH oxidase with tethered NAD/NADH and a galactose oxidase, such as galactose oxidase variant (GO_{M3-5}).

In another example, a first enzyme complex comprising glycerol-3-phosphate and NADH oxidase with tethered NAD/NADH is coupled with further enzymes such as a galactose oxidase, such as galactose oxidase variant (GO_{M3-5}) and/or an aldolase, such as ScFruA, EcTagA, EcFucA or EcRhuA.

In these examples, the other enzyme may be covalently attached to a conjugation module. For example, the other enzymes can include a galactose oxidase, such as galactose oxidase variant (GO_{M3-5}) covalently attached to an esterase such as Alicyclobacillus acidophilus esterase and/or an aldolase such as Staphylococcus carnosus aldolase (ScFruA), Escherichia coli Tagatose-biphosphate aldolase (EcTagA) or Escherichia coli Rhamnulose-1-phosphate aldolase (EcRhuA) covalently attached to Alicyclobacillus acidophilus esterase. For example, the other enzyme can be Staphylococcus carnosus aldolase (ScFruA) covalently attached to Alicyclobacillus acidophilus esterase (AaE2). In another example, the other enzyme can be Thermus caldophilus aldolase covalently attached to AaE2. Accordingly, in another example, an enzyme complex comprising TkG1pK::MaAk::AaE2 with tethered ATP/ADP is coupled with a further enzyme complex comprising EcG3PD::CaNOX::AaE2 with tethered NAD/NADH and another enzyme such as ScFruA::AaE2. In this example, aminocyclitol can be produced from glycerol and Cbz-aminopropanal.

One of skill in the art will be aware of various other applications for the enzyme complexes of the present disclosure. Examples include, reduction of enones by NAD(P)H-dependant enoate reductases; generation of chiral secondary alcohols by cofactor-dependant alcohol dehydrogenases and reductive amination to produce chiral amines by amino acid dehydrogenase. Other exemplary sugar analogues that can be produced using enzyme complexes according to the present disclosure include DNJ (1-deoxynojirimycin), DMJ (1-deoxymanojirimycin), Miglitol, Miglustat, DAB (1,4-dideoxy-1,4-imino-D-arabinitol), 5-DDAB (1,4,5-trideoxy-1,4-imino-D-arabitol), D-fagomine, DMDP (2,5-dideoxy-2,5-imino-D-mannitol).

In another example, the enzyme complexes of the present disclosure can be used in bioreactor such as a continuous flow bioreactor for large scale cofactor-dependant biocatalytic syntheses. Various suitable bioreactors are known in the art (see, for example, Mazid et al., 1993).

In an example, the present disclosure encompasses a bioreactor comprising a reservoir of substrate in solution and a first reaction cell comprising an enzyme complex according to the present disclosure, wherein the first reaction cell is in fluid communication with the reservoir. In an example, the bioreactor further comprises a second reaction cell comprising an enzyme complex of the present disclosure, wherein the second reaction cell is in fluid communication with the first reaction cell. In an example, the bioreactor further comprises additional reactions cells comprising an enzyme complex of the present disclosure, wherein each additional reaction cell is in fluid communication with the previous reaction cell. In an example, circulating free cofactor is added to the bioreactor. In another example, additional substrate is added to the bioreactor. One of skill in the art will appreciate that various additional substrates can be added to dictate production of the final product. For example, an additional substrate can be supplied to a reaction mixture containing DHAP and an aldolase to produce various chiral sugars. In an example, the additional substrate is Cbz-aminopropanal.

In another example, the reaction cell comprises a solid support exemplified above. For example, the reaction cell can comprise a polysaccharide with primary hydroxyl groups available for chemical modification such as agarose beads or cotton.

In an example, the reaction cell comprises a cotton disc. In an example, the bioreactor comprises a pump to provide continuous flow of solution from the reservoir through each reaction cell.

In another example, the enzyme complexes of the present disclosure can be used for screening applications in drug discovery by providing a simple means to generate a vast array of chiral sugars and other relevant molecules.

In another example, the enzyme complexes of the present disclosure can be used in bioremediation by providing a means to utilise cofactor-dependant enzymes in bioremediant situations without the problematic issues of expensive provision of large amounts of cofactor.

### EXAMPLES

### Example 1—Construction and Demonstration of Bi-Enzymatic Fusion Proteins

22 enzymes were assessed for the synthesis steps of DHAP from glycerol (regiospecific phosphorylation and oxidation) with appropriate cofactor recycling. The four best enzyme combinations were then used to synthesise bi-enzymatic fusion proteins. Each fusion protein produced is a single molecule that encodes two functionalities, a DHAP-synthetic step and cognate cofactor recycling.

Bi-enzymatic fusion proteins were produced by fusing the genes encoding the relevant enzymes with a short synthetic region of DNA that encoded an amino acid linker comprising GlySerSer repeats (GSS)ₙ with a cysteine in the middle of the linker for later incorporation of the modified cofactor i.e. (GSS)₃C(GSS)₃.

Bi-enzymatic fusion 1 (BiF1) contains the optimal enzymes for glycerol-3-phosphate production and ATP regeneration (Thermococcus kodakarensis glycerol kinase [TkG1pK] and Mycobacterium smegmatis ATP kinase [MsAK]).

Bi-enzymatic fusion 2 (BiF2) contains the optimal enzymes for DHAP production from glycerol-3-phosphate and regeneration of NAD (Escherichia coli glycerol-3-phosphate dehydrogenase [EcG3PD] and Clostridium aminoverlaricum NADH oxidase [CaNOX]).

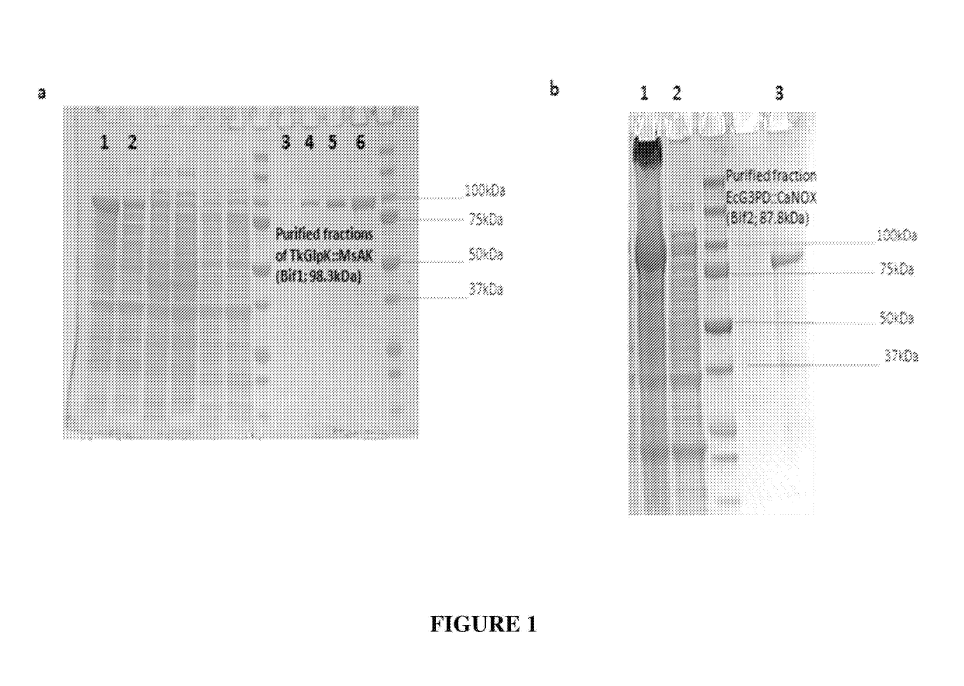

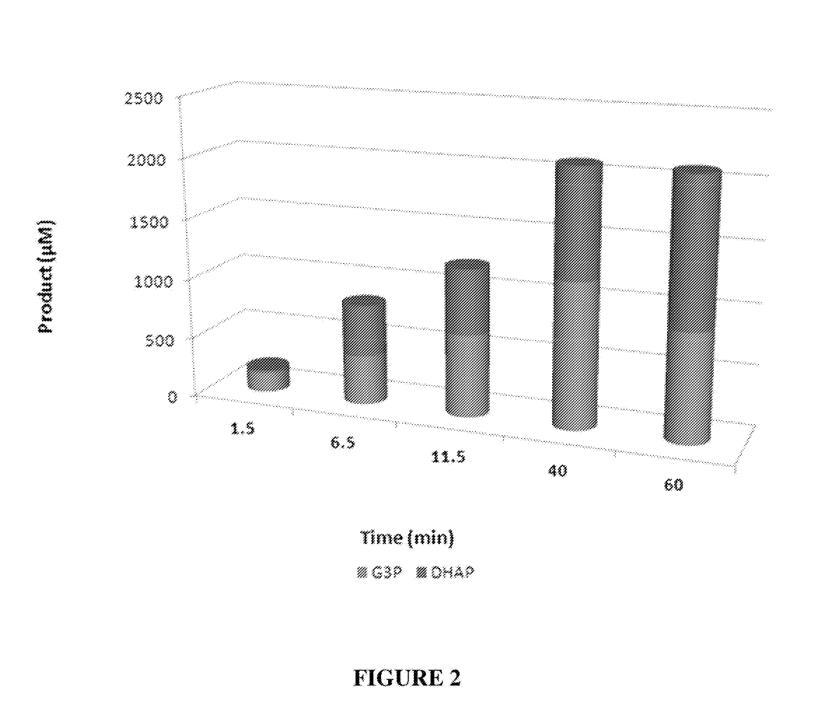

Expression of soluble bi-enzymatic fusion protein in E. coli cells was optimised by varying induction temperature, strain of E. coli, amount of inducer and time of induction. The optimal expression conditions for both constructs comprised induction with 1 mM IPTG at 15° C. overnight in E. coli; an example of BiF expression and purification is shown in FIG. 1.

The functionality of the purified bi-enyzymatic fusion proteins BiF1 and BiF2 was assessed (Tables 4 and 5). BiF1 was shown to be able to produce glycerol-3-phophsate from glycerol with similar efficiency to the glycerol kinase component enzyme alone, and also to efficiently recycle ADP to ATP, albeit with a higher K_{M} requirement for the acetyl phosphate regeneration co-substrate (Table 5). BiF2 was purified and shown to be able to produce DHAP from glycerol-3-phosphate. BiF2 demonstrated efficient recycling of NADH to NAD⁺, albeit at a slightly slower rate than the CaNOX cofactor-recycling enzyme alone. However, the catalytic rate of the EcG3PD component of BiF2 was considerably slower than EcG3PD enzyme alone and the Kₘ for glycerol-3-phosphate increased somewhat, resulting in a log decrease in catalytic efficiency K_{cat}/Kₘ (Table 6).

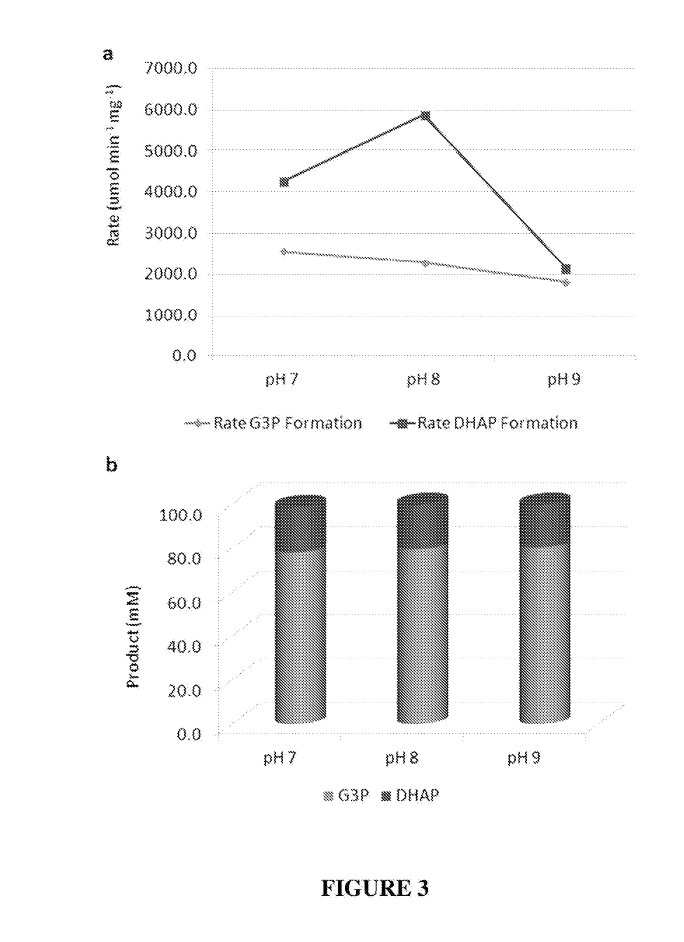

DHAP production from batch reactions containing BiF1 and BiF2 was successful under a variety of conditions. The combined bi-enzymatic fusions were able to consume 2 mM glycerol in one hour and convert it to a mixture of glycerol-3-phosphate and DHAP (FIG. 2), and catalyse ˜90% conversion of 100 mM glycerol to glycerol-3-phosphate and DHAP after 18 hours in a scaled up batch reaction (FIG. 3).

Batch reactions based on the fused enzymes perform as well as batch reactions based on the non-fused enzymes (Tables 7 and 8). However, overall yield of DHAP from glycerol in the bi-enzymatic batch reactions was limited by product inhibition of the glycerol-3-phopshate dehydrogenase enzyme component by DHAP (Kᵢ˜0.1 mM). This resulted in yields of DHAP of ˜63% and ˜22% from the 2 mM and 100 mM glycerol batch reactions, respectively (FIGS. 2 and 3).

TABLE 5
Efficiency of bi-enzymatic fusion protein BiF1 for conversion of glycerol to
glycerol-3-phosphate (G3P)
Glycerol Kinase Activity

| | | K_{M} | K_{M} | | | | |
|---|---|---|---|---|---|---|---|
| | | (glycerol; | (ATP; | | K_{cat}/K_{M} | pH | pH |
| Design # | Source | μM) | μM) | K_{cat} (s⁻¹) | (M⁻¹s⁻¹) | Optima | Range |
| BiF1 | TkGK-MsAK 1 | 14.5 ± 4 | 123 ± 21 | 1125 ± 115 | 7.7 * 10⁷ | 8.5 | 6-10 |

ATP Kinase Activity

| | | | K_{M} | | K_{cat}/K_{M} | pH | pH |
|---|---|---|---|---|---|---|---|
| Design # | Source | K_{M} (ADP) | (AcP) | K_{cat} (s⁻¹) | (M⁻¹s⁻¹) | Optima | Range |
| BiF1 | TkGK-MsAK 1 | 424 ± 35 | 1400 ± 126 | 759 ± 53 | 542 | 7.5 | 6-10 |

TABLE 6
Efficiency of bi-enzymatic fusion protein BiF2 for conversion of glycerol-3-
phosphate to DHAP
Glycerol-3-phosphate Dehydrogenase Activity

| | | K_{M} | K_{M} | | K_{cat}/ | | |
|---|---|---|---|---|---|---|---|
| | | (G3P; | (NAD; | | K_{M} | pH | pH |
| Design. # | Source | μM) | μM) | K_{cat} (s⁻¹) | (M⁻¹s⁻¹) | Optima | Range |
| BiF2 | EcG3PD- | 369 ± 17 | 176 ± 12 | 6.8 ± 0.7 | 2.6 * 10⁴ | 9.0 | 7-9.5 |
| | CaNOX1 | | | | | | |
| | (BiF2) | | | | | | |

NADH oxidase Activity

| | | | | K_{cat}/ | | |
|---|---|---|---|---|---|---|
| | | K_{M} | | K_{M} | pH | pH |
| Design. # | Source | (NADH) | K_{cat} (s⁻¹) | (M⁻¹s⁻¹) | Optima | Range |
| BiF2 | EcG3PD- | 276 ± 9 | 1714 ± 252 | 3.9 * 10⁶ | 6 | 5-9 |
| | CaNOX 1 | | | | | |
| | (BiF2) | | | | | |

TABLE 7
Comparison of glycerol-3-phosphate and DHAP production efficiencies of
batch reactions using either four unfused enzymes or a combination of BiF1 and BiF2.

| | | | | | Rate G3P | Rate DHAP |
|---|---|---|---|---|---|---|
| Glycerol | ATP | G3P | NADH | % Total | Production | Production |
| Kinase | Kinase | dehydrogenase | Oxidase | Conversion^{#} | (μM⁻¹s⁻¹) | (μM⁻¹s⁻¹) |
| TkGK2 | Ms AK1 | EcG3PD2 | Ca NOX1 | 29 ± 0.7 | 1.24 ± 0.4 | 1.66 ± 0.5 |

| | | | | |
|---|---|---|---|---|
| BiF1 | BiF2 | 21 ± 0.9 | 1.21 ± 0.3 | 1.44 ± 0.4 |

TABLE 8
Relative efficiencies of glycerol-3-phosphate dehydrogenase enzymes and
NADH oxidase (NOX) enzymes with modified cofactor.

| | | | | | K_{cat/}K_{M} |
|---|---|---|---|---|---|
| Design. # | Source | Substrate | K_{M} (μM) | K_{cat} (s⁻¹) | (M⁻¹s⁻¹) |
| EcG3PD2 | E. coli | NAD | 147.1 ± 25.4 | 66.7 ± 10.6 | 4.5 * 10⁵ |
| | | N⁶-2AE-NAD | 181.2 ± 37.5 | 63.5 ± 9.4 | 3.5 * 10⁵ |
| CaNOX | C. aminoverl | NADH | 204 ± 15.4 | 1204 ± 67.5 | 5.9 * 10⁶ |
| | aricum | | | | |
| | | N⁶-2AE-NADH | 215 ± 27.2 | 343 ± 32.6 | 1.6 * 10⁶ |

Tables 4-7#

Reactions were conducted at room temperature in 1 mL total volume with 10 mM glycerol as starting substrate, between 1 and 14 nM of enzyme and 100 μM each of ATP and NAD. Samples were collected at various time points and analysed by LCMS (SIM monitoring for G3P and DHAP).

As outlined below, addition of an aldolase enzyme to the batch reaction for conversion of DHAP to sugars or sugar analogues provides a mechanism to prevent accumulation of product, reducing DHAP-mediated product inhibition of glycerol-3-phosphate dehydrogenase. Furthermore, incorporating BiF1 and BiF2 into the intended flow reactor also alleviates the inhibitory effect observed in the batch reactor.

The turnover numbers for the cofactors (i.e. how many times each cofactor molecule was used and recycled) were also obtained. The turnover number of the ATP cofactor involved in the redox reactions was excellent, achieving close to the maximum possible total of 200 turnovers of ATP per batch reaction (90 mM conversion of glycerol to glycerol-3-phosphate from 0.1 mM ATP starting concentration; ˜40/hour). This level is approaching commercial industry standard turnover frequencies (TOF) of 1000 per hour (Rocha-Martin et al., 2012).

Turnover of the NAD⁺ cofactor is less easily assessed in a contained batch reactor format, as product inhibition of the G3P-dehydrogenase reaction limits possible turnover. Nonetheless the initial rate of NAD⁺ turnover (22 per ten minutes) can be extrapolated to ˜132 per hour.

The effect of pH from 5-10 on the glycerol to DHAP (BiF1 plus BiF2) reactions was assessed with 100 mM glycerol substrate. There was very little difference in the initial rate of G3P formation, and a slightly increased rate of DHAP formation at pH 8 (FIG. 3). This is consistent with a mid-point of the optima for the synthetic and cofactor recycling enzymes involved (EcG3PD, pH 9 and CaNOX, pH 7). However, it should be noted that changing pH produced no significant difference in overall conversion and yield of DHAP when the reaction was left to run to completion overnight (FIG. 3).

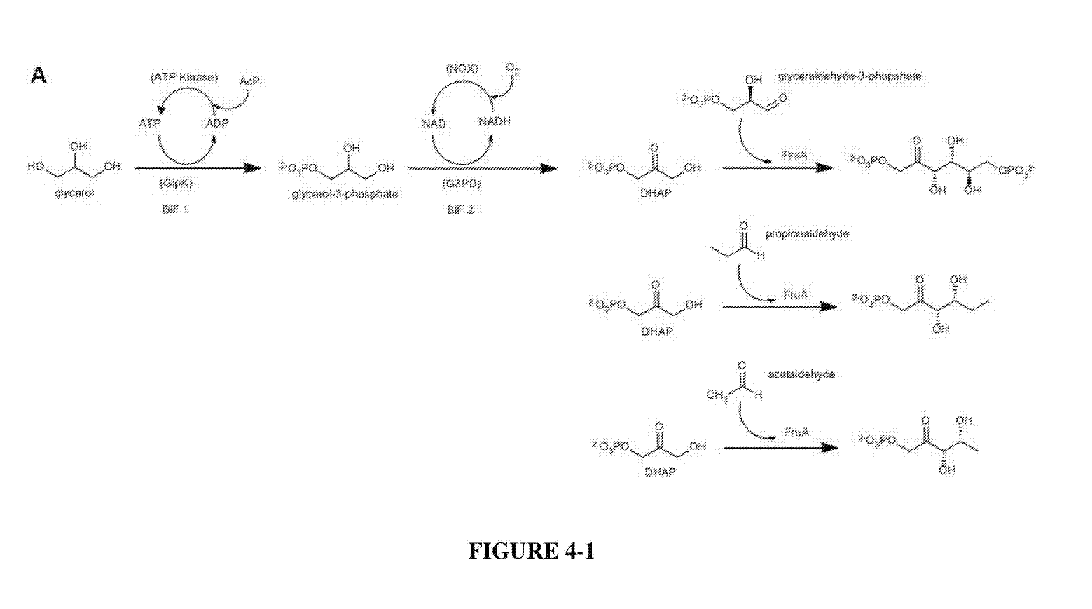

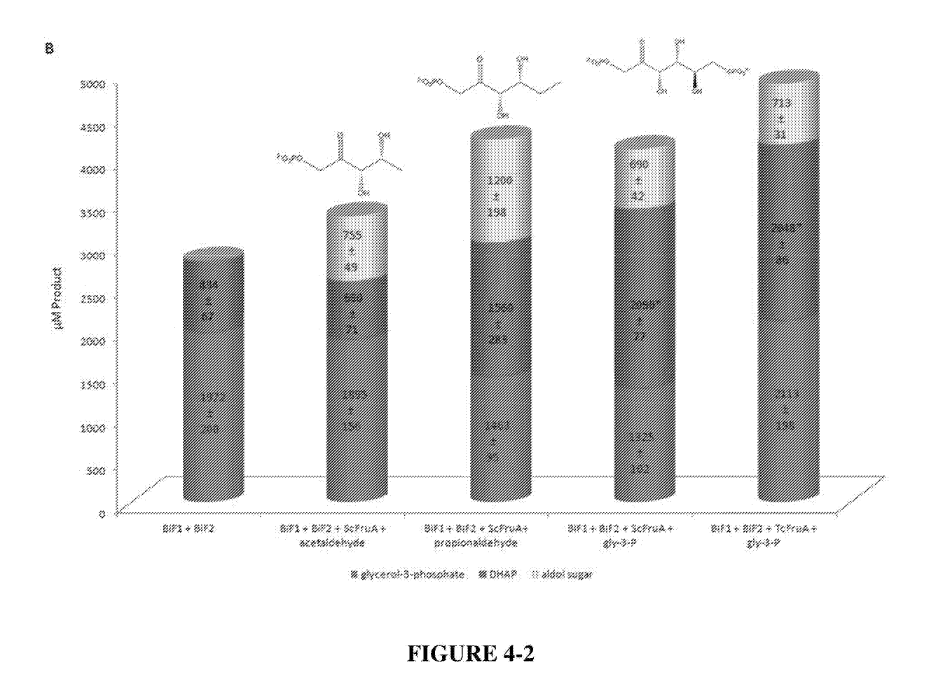

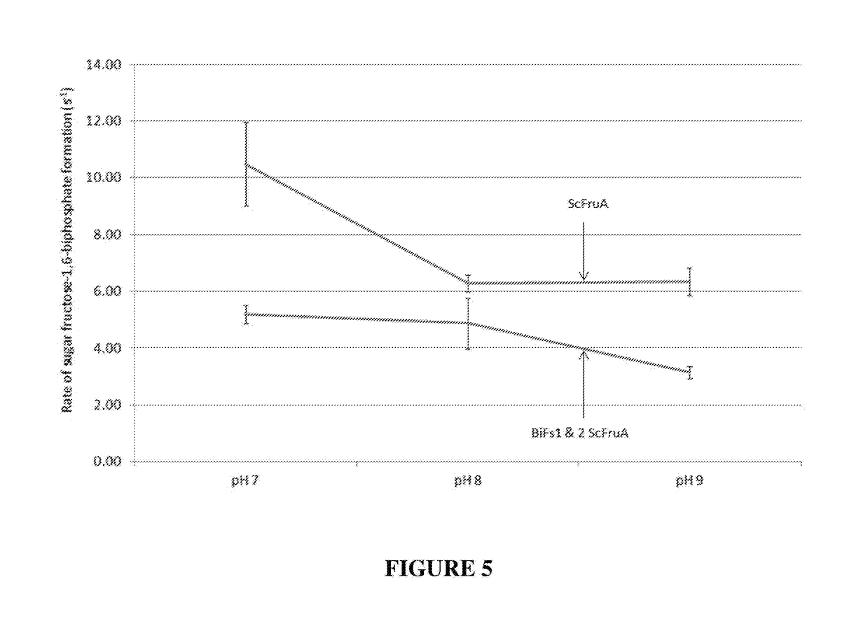

Finally, the BiF1+BiF2 production of DHAP was coupled with two stereospecific DHAP-dependant aldolases for the production of sugars from glycerol. BiF1 and BiF2 fusion enzymes were combined with aldolases from both S. carnosus I (Witke and Gotz, 1993) and from T. caldophilus (thermostable; (Lee et al., 2006)), and successfully produced sugars via aldol condensation when combined with three different aldehyde acceptors: acetaldehyde and propionaldehyde produced unnatural sugars and glyceraldehyde-3-phosphate produced the natural product for these enzymes (FIG. 4). BiFs 1 and 2 were first reacted with glycerol for thirty minutes before addition of aldolase enzymes, and then reacted for a further one hour. The optimum pH for the multi-enzyme batch reactions was shown to be between pH 7-8 (FIG. 5), congruent with the optimum pH for the aldolase reaction (pH 7, FIG. 5) and combined BiF reaction (pH 8, FIG. 3a).

### Cofactor Functionalisation

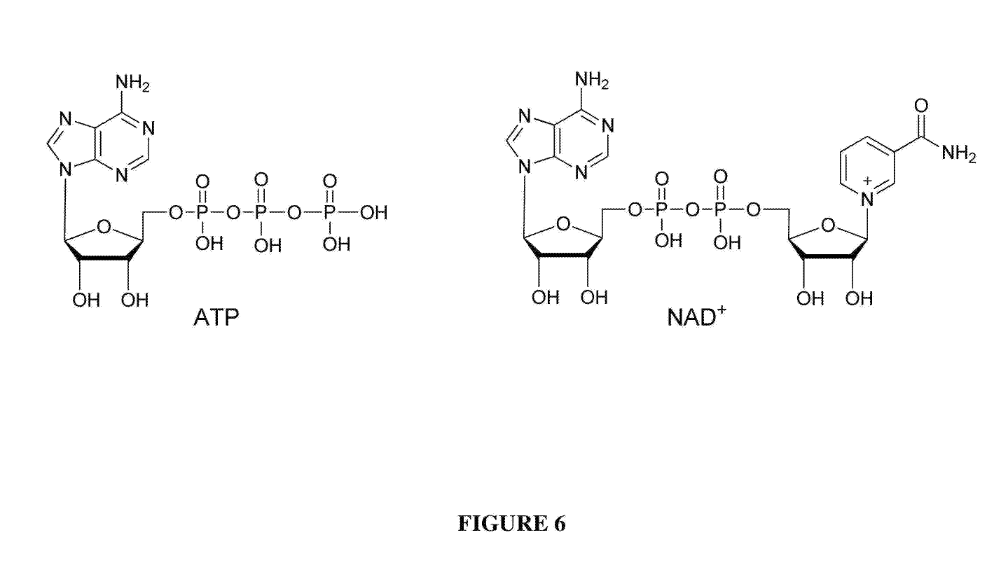

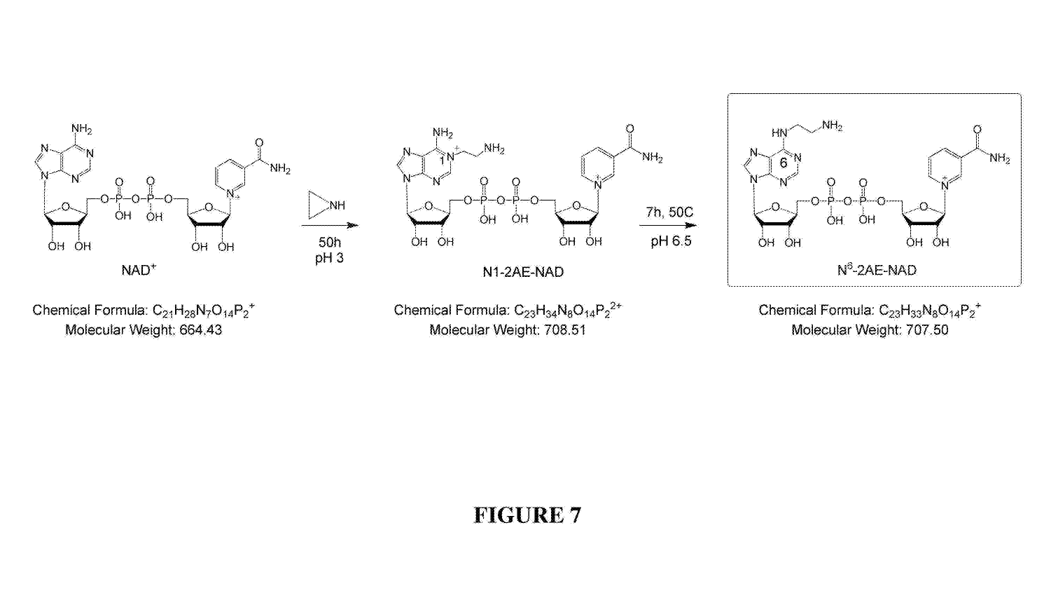

Cofactors were functionalised for tethering to BiF fusions to allow retention of the factor in the flow cell and in proximity to the BiF fusions. Various cofactors such as NAD and ATP contain a common ribonucleotide ‘core’ (FIG. 6). The ribonuclotide core can be used as the site of functionalisation (FIG. 7).

The following is directed towards functionalisation of NAD but is theoretically applicable for functionalisation of other cofactors with a ribonucleotide core.

NAD was alkylated (aziridine alkylation) to produce an N1-2AE-NAD intermediate. It was unnecessary to separate unreacted NAD from the N1-2AE-NAD/NAD mixture to be able to transform it to an N⁶-2AE-NAD/NAD mixture. Accordingly, this mixture was directly reacted with a cross-linker containing an NHS ester, or CO₂H at one end. The lack of reactivity of NAD lead to complete reaction of the cross-linker with N⁶-2AE-NAD.

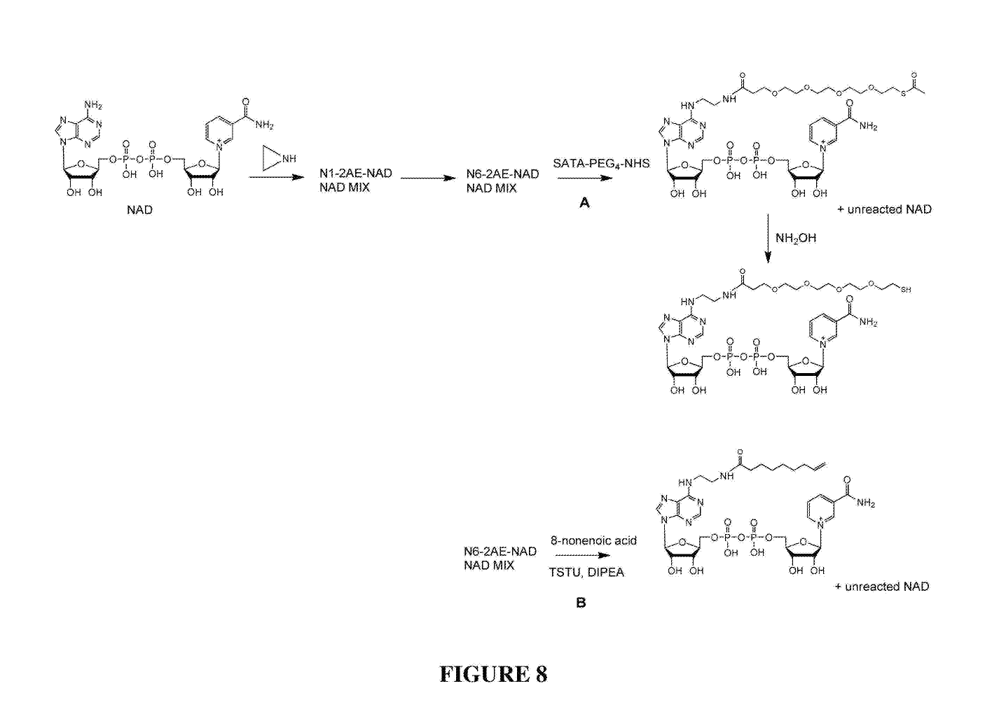

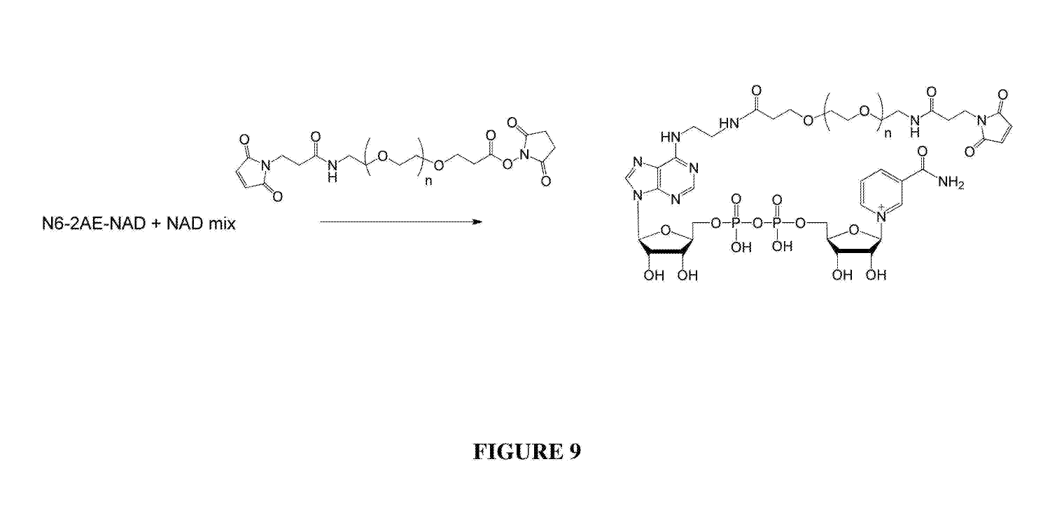

To this end N⁶-2AE-NAD was reacted with both SATA-PEG₄-NHS (FIG. 8A, SATA (N-succinimidyl S-acetylthioacetate)) or MAL-PEG₂₄-NHS (FIG. 9) or 8-nonenoic acid (FIG. 8B, under amide coupling conditions) to yield the resulting tethered constructs which both have a retention time by HPLC that is significantly different to NAD thus isolation by HPLC was straightforward.

PEG and hydrocarbon linkers were attached to NAD. This demonstrates the ability to install both hydrophilic (PEG) and hydrophobic (hydrocarbon) linkers by the use of either an NHS active ester or ester formed in situ from a CO₂H and peptide coupling agents. Both of the tethers installed have a reactive functional group at the opposing end for further conjugation to an enzyme complex or surface.

For example, when using a cysteine as an immobilisation point in the enzyme, NAD-2AE-(CH₂)₆—CH═CH₂-can be installed via thiolene chemistry at a cysteine thiol residue. Alternatively, a PEG linker with a terminal maleimide can be easily prepared from available materials (FIG. 9), this NAD-2AE-PEGx-MAL construct can be used to install NAD via a Michael addition reaction to the cysteine thiol residue on the enzyme fusion complex.

A suitably modified NAD-2AE-PEGx-MAL was also produced (FIG. 9).

The relative enzyme activity for the NAD-dependant glycerol-3-phosphate dehydrogenase enzymes identified for DHAP synthesis was assessed with the modified N⁶-2AE-NAD. Kinetic data for EcG3PD and CaNOX was also obtained. To determine the relative activities and kinetic enzyme efficiency, modified N⁶-2AE-NAD was reduced enzymatically, separated from enzymes using ultrafiltration and the amount of N⁶-2AE-NADH calculated based on the absorbance A₃₄₀ₙₘ. These data indicate that modification of the N⁶ position of NAD produced a cofactor analogue that was still biochemically active (i.e. it was accepted by enzymes and could participate in redox reactions).

The full kinetic analysis shows that glycerol-3-phosphate dehydrogenase 2 (EcG3PD) retains 78% of activity with the modified cofactor compared with unmodified NAD. There is a slight increase in binding affinity (K_{M}), and a slight decrease in catalytic efficiency (K_{cat}), but overall very little significant difference in the catalytic constant.

In contrast, however, there was a reduction in the catalytic efficiency of the NOX1 (CaNOX) enzyme with the modified N⁶-2AE-NADH compared to NADH as substrate. However, the high initial catalytic efficiency of NOX1 means that this reduction in activity should not be rate-limiting in the molecular machine as the reduced activity is still greater than the catalytic efficiency of glycerol-3-phosphate dehydrogenase 2. Hence, cofactor oxidation should still be considerably more rapid than the catalytic conversion of glycerol-3-phosphate and the concomitant cofactor reduction.

### Construction and Demonstration of Functional Cofactor-Tethered Bi-Enzymatic Fusion Protein

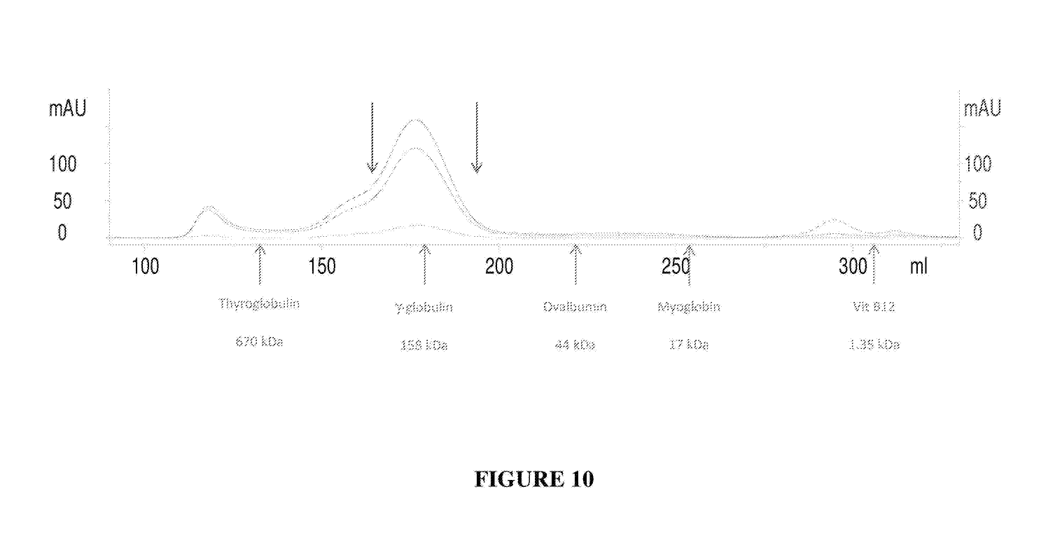

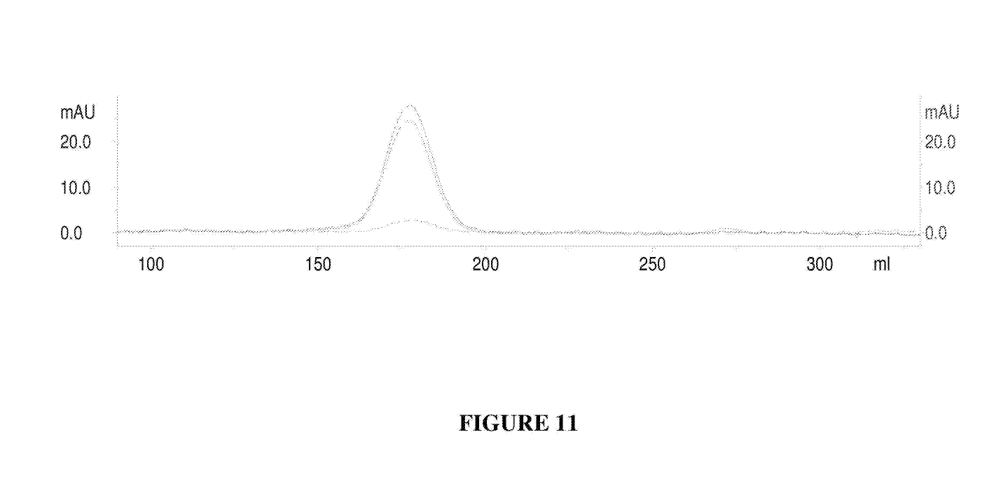

The chromatogram of BiF2 (EcG3PD-CaNOX) shows the peak of protein elutes at 177 mL, which is consistent with a dimer MW of 176 kDa (FIG. 10). The NADH oxidase has a bound FAD which contributes to the absorbance at 450 and 259 nm. To prevent undesired side reactions, TCEP was removed from the pool by desalting immediately prior to the addition of one equivalent of NAD-2AE-PEG₂₄-MAL. The gel filtration profile of the NAD-2AE-PEG₂₄-BiF2 conjugate shows an increase in the absorbance at 259 nm relative to the protein absorbance at 280 nm, consistent with the presence of the NAD (FIG. 11). There is no evidence for unconjugated NAD-2AE-PEG₂₄-MAL eluting at the end of the run, consistent with the majority of the NAD being tethered to the BiF2.

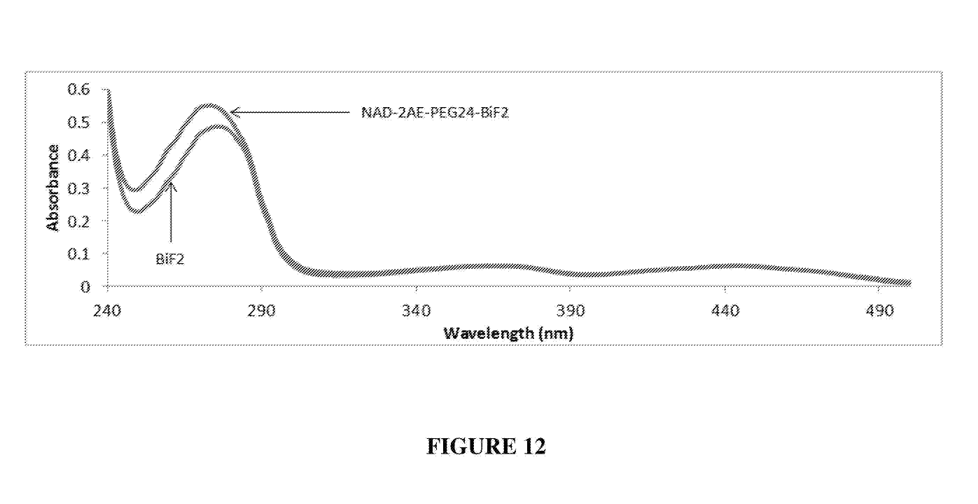

The UV-vis spectra of BiF2 and NAD-2AE-PEG₂₄-BiF2 conjugate have peaks at 360 and 450 nm, consistent with the presence of bound NAD (FIG. 12). The conjugate has a peak of absorbance at 273 nm which is higher than the peak for BiF2 at 276 nm, which is consistent with the presence of NAD in the conjugate.

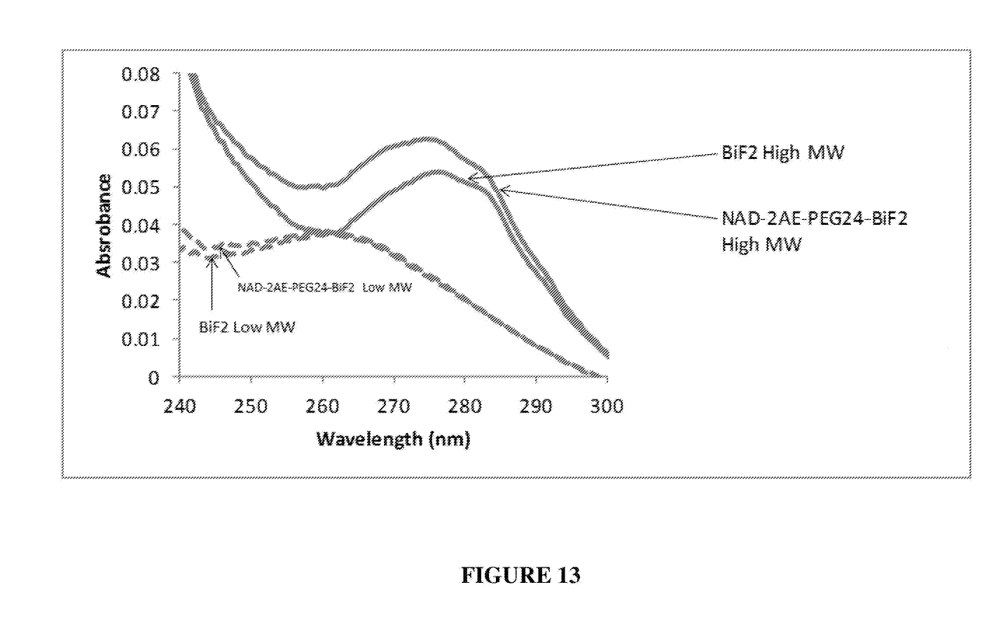

Non-covalently linked cofactor was separated from the complex by denaturation in GuHCl and ultrafiltration to separate the low molecular weight cofactor from the protein. The UV-vis spectra of the separated low MW material was very similar for both BiF2 and NAD-2AE-PEG₂₄-BiF2, which is consistent with both protein and conjugate having non-covalently linked NAD (FIG. 13). The high MW spectra show the conjugate has a higher absorbance at 260 nm, which is consistent with the presence of covalently tethered NAD cofactor.

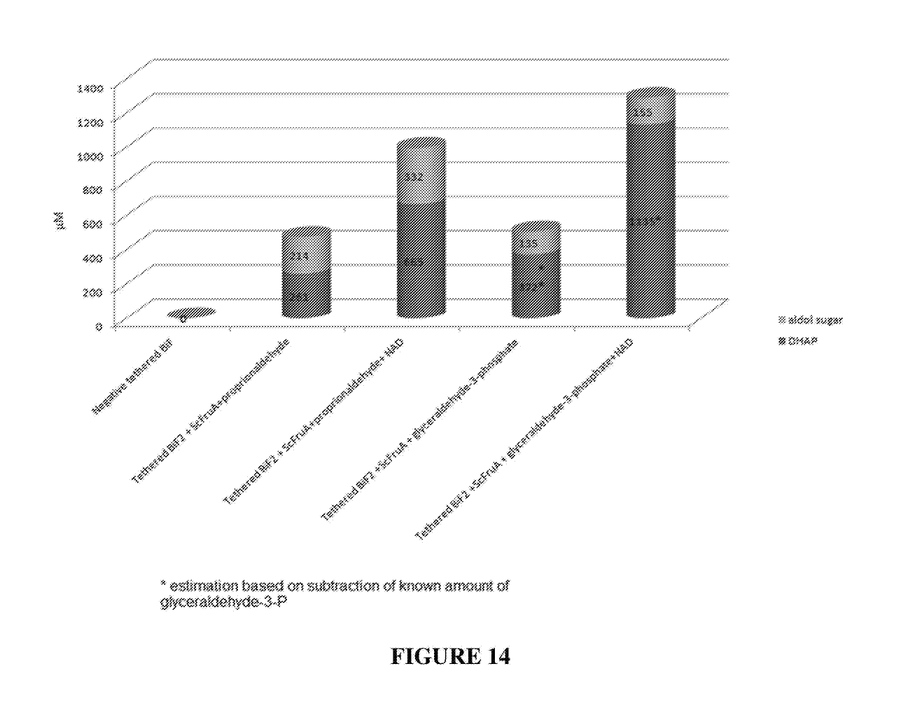

Due to the unstable nature of DHAP in solution, the production of DHAP by the nanomachine biocatalyst was further verified by combination of cofactor-tethered BiF2 reaction products with aldolase enzyme ScFruA and an aldehyde acceptor co-substrate to demonstrate DHAP-dependant production of aldol sugars (FIG. 14). Once again this confirmed that the cofactor-tethered BiF2 fusion protein was able to produce sufficient DHAP to allow DHAP-dependant ScFruA aldol condensation reactions to occur with both propionaldehyde and glycerol-3-phosphate aldehyde acceptors.

Thus, the cofactor-tethered bienzymatic fusion proteins described herein are capable of functioning as nanomachine biocatalysts to convert glycerol-3-phosphate to DHAP without addition of exogenous cofactor. Further, they can be coupled with, for example, an aldolase enzyme to produce a variety of chiral molecules.

Construction and Demonstration of Functional Cofactor-Tethered Tri-Enzymatic Fusion Proteins and Conjugation onto a Solid Surface

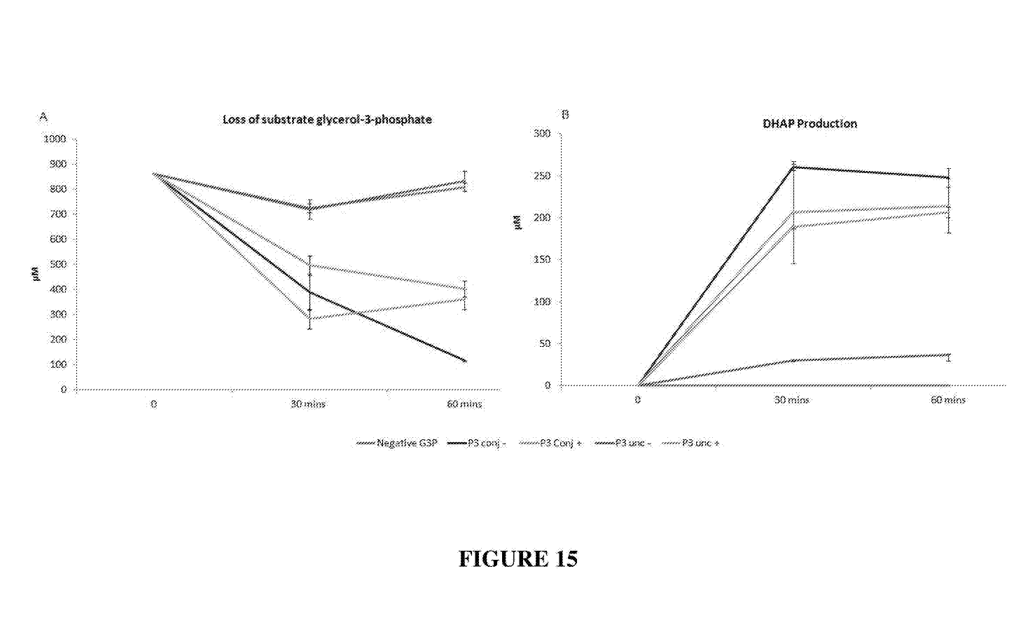

A “conjugation module” protein, an esterase enzyme from Alicyclobacillus acidophilus, denoted Alicyclobacillus acidophilus esterase, was incorporated into BiF1 and BiF2 proteins via genetic fusion with each BiF to produce trienzymatic fusion protein 1 (TkG1pK-MaAk-Alicyclobacillus acidophilus esterase; TriF1, 132 kDa) and trienzymatic fusion protein 2 (EcG3PD::CaNOX::Alicyclobacillus acidophilus esterase; TriF2, 124 kDa) (FIG. 15), Table 9).

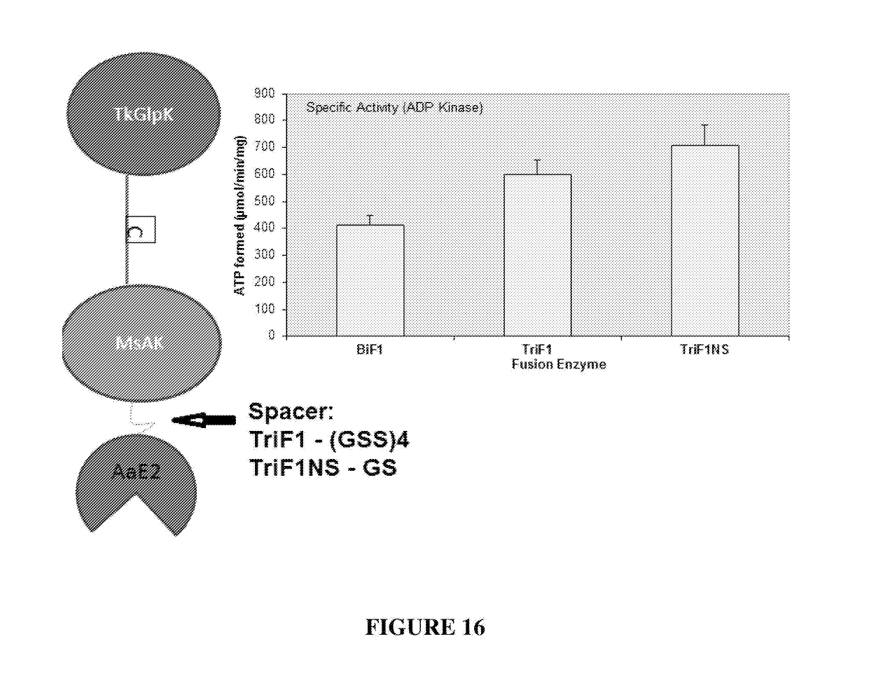

Two different variants of TriF1 were produced in order to assess the effect of different linker lengths between the bienzymatic fusion protein and the esterase component of the final trienzymatic fusion protein. A very short linker region (gly-ser) was shown to a produce slightly more active fusion protein (TriF1-NS), versus a longer linker region (gly-ser-ser)₄; TriF1) (FIG. 16), although there was no detectable difference in protein expression. TriF1-NS was used for all subsequent experiments and for simplicity is hereafter referred to as TriF1.

The functionality of the component enzymes of purified TriF1 and TriF2 were assessed and compared with the non-fused and bi-enzymatic fusion activities of these enzymes (Tables 3 and 4).

TriF1 was shown to be able to produce glycerol-3-phosphate from glycerol with similar efficiency to the glycerol kinase component enzyme alone, and also to efficiently recycle ADP to ATP, albeit with a higher K_{M} requirement for the acetyl phosphate regeneration of co-substrate (Table 9). TriF2 was purified and shown to be able to produce DHAP from glycerol-3-phosphate. TriF2 demonstrated efficient recycling of NADH to NAD⁺, albeit at a slightly slower rate than the CaNOX cofactor-recycling enzyme alone. However, the catalytic rate of the EcG3PD component of TriF2 was considerably slower than EcG3PD enzyme alone and the Kₘ for glycerol-3-phosphate increased somewhat, resulting in a log decrease in catalytic efficiency K_{cat}/Kₘ (Table 10).

TABLE 9
Efficiency of Tri-Enzymatic fusion protein TriF1 for conversion of glycerol
to glycerol-3-phosphate.
Glycerole Kinase Activity

| | | K_{M} | K_{M} | | K_{cat}/K_{M} | | |
|---|---|---|---|---|---|---|---|
| | | (glycerol; | (ATP; | | (M⁻¹s⁻¹) | pH | pH |
| | Source | μM) | μM) | K_{cat} (s⁻¹) | (glycerol) | Optima | Range |
| GlpK2 | TkGlpK | 15.4 ± 2 | 111 ± 12 | 940 ± 8 | 6.1 * 10⁷ | 8.5 | 7.0-9.5 |
| GlpK2 | TkGK-MsAK | 14.5 ± 4 | 123 ± 21 | 1125 ± 115 | 7.7 * 10⁷ | 8.5 | 6-10 |
| | (BiF1) | | | | | | |
| GlpK2 | TkGK-MsAK- | 16.3 ± 4 | 115 ± 19 | 1399 ± 54 | 8.6 * 10⁷ | 8.5 | 6-10 |
| | Alicyclobacillus | | | | | | |
| | acidophilus | | | | | | |
| | esterase | | | | | | |
| | (TriF1) | | | | | | |

ATP Kinase Activity

| | | | | | K_{cat}/K_{M} | | |
|---|---|---|---|---|---|---|---|
| | | K_{M} (ADP; | K_{M} | | (M⁻¹s⁻¹) | pH | pH |
| | Source | μM) | (AcP) | K_{cat} (s⁻¹) | (AcP) | Optima | Range |
| AK1 | Ms AK | 113 ± 9 | 390 ± 8 | 1103 ± 126 | 2.8 * 10⁶ | 7.5 | 6-10 |
| AK1 | TkGK-MsAK | 424 ± 35 | 1400 ± 126 | 759 ± 53 | 5.4 * 10⁵ | 7.5 | 6-10 |
| | (BiF1) | | | | | | |
| AK1 | TkGK-MsAK- | 398 ± 29 | 1197 ± 114 | 1084 ± 37 | 9.1 * 10⁵ | 7.5 | 6-10 |
| | Alicyclobacillus | | | | | | |
| | acidophilus | | | | | | |
| | esterase | | | | | | |
| | (TriF1) | | | | | | |

TABLE 10
Efficiency of Tri-Enzymatic fusion protein TriF2 for conversion of glycerol-
3-phosphate to DHAP.
Glycerol-3-phosphate Dehydrogenase Activity

| | | | | | K_{cat}/ | | |
|---|---|---|---|---|---|---|---|
| | | K_{M} | K_{M} | | K_{M} | | |
| | | (G3P; | (NAD; | | (M⁻¹s⁻¹) | pH | pH |
| Design. # | Source | μM) | μM) | K_{cat} (s⁻¹) | (G3P) | Optima | Range |
| G3PD2 | EcG3PD | 59 ± 4 | 158 ± 24 | 85 ± 11 | 1.4 * 10⁶ | 9.0 | 7-9.5 |
| G3PD 2 | EcG3PD-CaNOX1 | 369 ± 17 | 176 ± 12 | 6.8 ± 0.7 | 1.8 * 10⁴ | 9.0 | 7-9.5 |
| | (BiF2) | | | | | | |
| G3PD 2 | EcG3PD-CaNOX1 | 659 ± 47 | 164 ± 10 | 7.1 ± 0.6 | 1.1 * 10⁴ | 9.0 | 7-9.5 |
| | (TriF2) | | | | | | |

NADH oxidase Activity

| | | | | K_{cat}/ | | |
|---|---|---|---|---|---|---|
| | | K_{M} | | K_{M} | pH | pH |
| Design. # | Source | (NADH) | K_{cat} (s⁻¹) | (M⁻¹s⁻¹) | Optima | Range |
| NOX 1 | CaNOX | 258 ± 21 | 1252 ± 182 | 4.9 * 10⁶ | 7.0 | 5-9 |
| NOX 1 | EcG3PD-CaNOX 1 | 276 ± 9 | 1714 ± 252 | 6.2 * 10⁶ | 6.0 | 5-9 |
| | (BiF2) | | | | | |
| NOX 1 | EcG3PD-CaNOX1 | 266 ± 15 | 1224 ± 114 | 4.6 * 10⁶ | 7.0 | 5-9 |
| | (TriF2) | | | | | |

The thermal stability of TriF1 and TriF2 in comparison to their native enzymes and bienzymatic fusion proteins was examined over a range of temperature from 40° C. to 100° C.

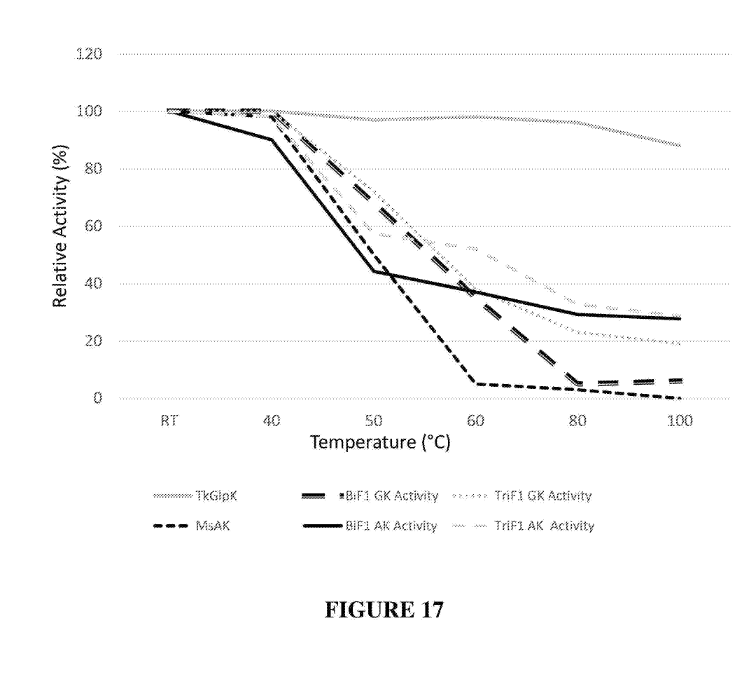

The glycerol kinase enzyme [TkG1pK] used in BiF1 and TriF1 (from T. kodakarensis) has high thermal stability. However, TkG1pK is destabilised when fused with the ATP kinase enzyme [MsAK] from M. smegmatis. The stability of BiF1 resembles that of MsAK with slightly increased residual activity at temperatures greater than 50° C. TriF1 follows a similar pattern but is in fact slightly more stable at temperatures up to 60° C. (FIG. 17).

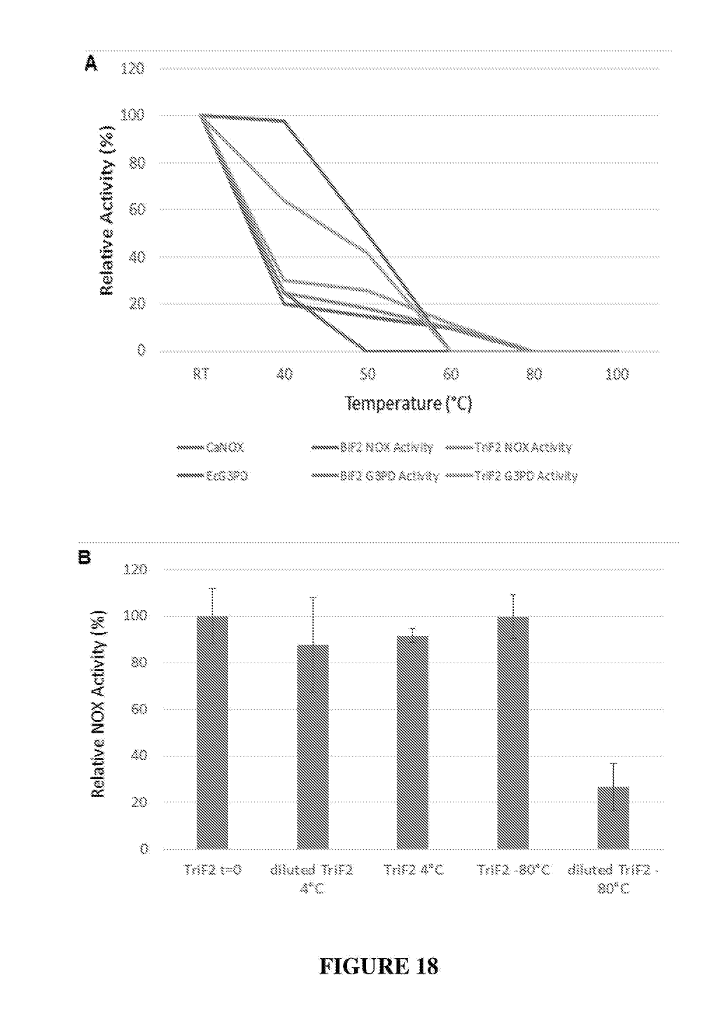

Both NADH oxidase and glycerol-3-phosphate dehydrogenase activities in BiF2 and TriF2 were slightly more stable as a fusion protein than their unfused counterparts (FIG. 18).

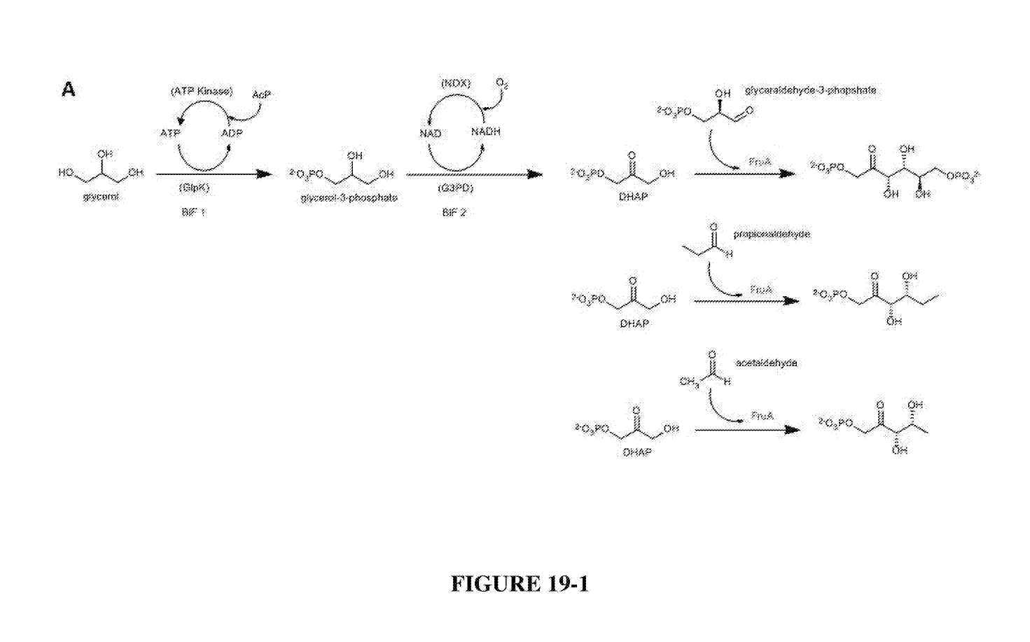

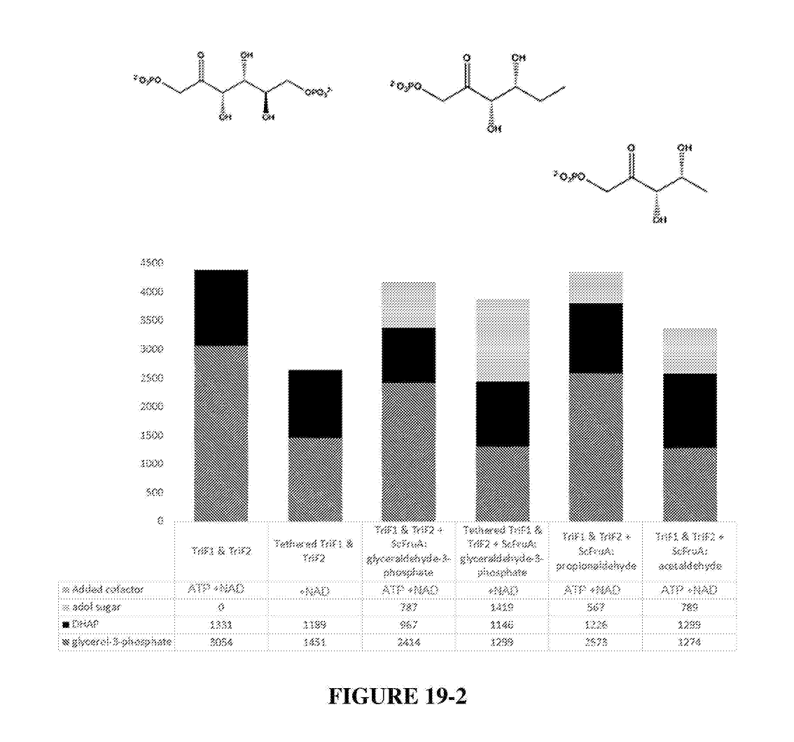

DHAP production from batch reactions containing TriF1 and TriF2 was successfully demonstrated under a variety of conditions. The combined tri-enzymatic fusions were able to consume 2 mM glycerol in one hour and convert it to a mixture of glycerol-3-phosphate and DHAP (Table 11), and catalyse a ˜50% conversion of 10 mM glycerol to glycerol-3-phosphate and DHAP after 1 hour in scaled up batch reaction (FIG. 19).

However, overall yield of DHAP from glycerol in the bi-enzymatic batch reactions was still limited by product inhibition of the glycerol-3-phopshate dehydrogenase enzyme component by DHAP (Kᵢ˜0.1 mM). This resulted in yields of DHAP of ˜68% and ˜20% from the 2 mM and 10 mM glycerol batch reactions, respectively (FIG. 19). Batch reactions based on the fused enzymes perform as well as batch reactions based on the non-fused enzymes (Table 10).

The turnover numbers for the cofactors (i.e. how many times each cofactor molecule was used and recycled) were also obtained. The turnover number of the ATP cofactor involved in the redox reactions was excellent, achieving close to the maximum possible total of 450 turnovers of ATP per batch reaction (4.5 mM conversion of glycerol to glycerol-3-phosphate from 0.01 mM ATP starting concentration).

The initial rate of NAD⁺ turnover (22 per ten minutes) can be extrapolated to 132 per hour if product inhibition were not in effect.

TABLE 11
Comparison of G3P and DHAP production efficiencies of batch reactions
using either four unfused enzymes, a combination of BiF1 and BiF2 or a combination
of TriF1 and TriF2.

| | | | | | Rate G3P | |
|---|---|---|---|---|---|---|
| Glycerol | ATP | G3P | NADH | % Total | Production | Rate DHAP |
| Kinase # | Kinase | dehydrogenase | Oxidase | Conversion^{#} | (μMs⁻¹) | Production (μMs⁻¹) |
| TkGK2 | Ms AK1 | EcG3PD2 | Ca NOX1 | 42 ± 0.7 | 1.24 ± 0.4 | 0.66 ± 0.5 |

| | | | | |
|---|---|---|---|---|
| BiF1 | BiF2 | 64 ± 0.9 | 1.21 ± 0.3 | 0.75 ± 0.4 |
| TriF1 | TriF2 | 68 ± 0.6 | 1.69 ± 0.1 | 0.78 ± 0.4 |

^{#}Reactions were conducted at room temperature in 1 mL total volume with 2 mM glycerol as starting substrate, between 1 and 14 nM of enzyme and 100 μM each of ATP and NAD. Samples were collected after 60 minutes and analysed by LCMS (SIM monitoring for G3P and DHAP).

Finally, the TriF1 plus TriF2 production of DHAP was coupled with two of the aldolases described above for the production of sugars from glycerol. TriF1 and TriF2 fusion enzymes were combined with aldolases from both S. carnosus I and from T. caldophilus (thermostable), and successfully produced sugars via aldol condensation when combined with three different aldehyde acceptor (acetaldehyde and propionaldehyde produced unnatural sugars and glyceraldehyde-3-phosphate produced the natural product for these enzymes). The system of enzymes used provides a broad platform for the production of unnatural sugars and sugar analogues.

TriFs 1 and 2 were first reacted with glycerol for thirty minutes before addition of aldolase enzymes, and then reacted for a further one hour (FIG. 19).

Tethering of ATP-CM-C₆-PEG₂₄-Maleimide to TkG1pK:MsAK::Alicyclobacillus acidophilus Esterase (TriF1)

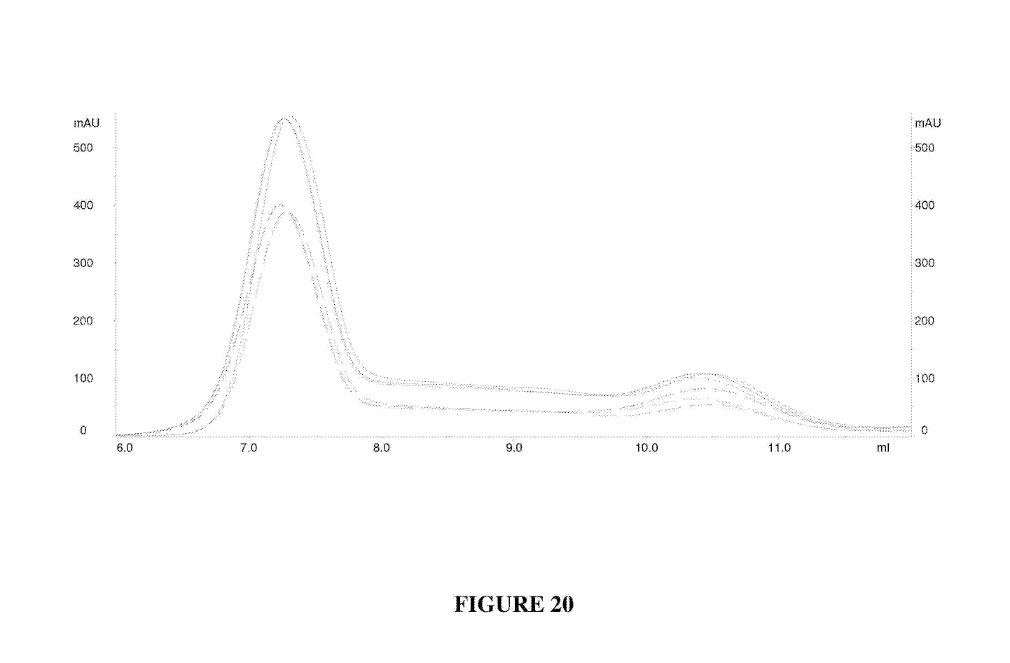

Gel filtration analysis of TriF1 showed the enzyme largely formed a soluble aggregate in solution, with only a small portion running at the expected elution volume (10.5 mL) for monomeric trifunctional fusion (FIG. 20). The enzyme was reacted with 10 equivalents of ATP-CM-C₆-PEG₂₄-maleimide in the presence or absence of 0.1 mM TCEP (FIG. 20). For the tethering in the presence of TCEP there was an increase of the A259 (dotted lines) relative to A280 (solid lines) for the monomeric TriF1, suggesting tethering of the ATP-CM-C₆-PEG₂₄-maleimide to the TriF1 was successful.

The remainder of TriF1 (20 mL, 34 mg, 0.26 μmol) was reacted with 10 equivalents of ATP-CM-C₆-PEG₂₄-maleimide (2.6 μmol) in the presence of 0.1 mM TCEP under the same conditions and used without further purification.

Glycerol-3-Phosphate Production by ATP-CM-C₆-PEG₂₄-MAL-TriF1 in the Absence of Added ATP

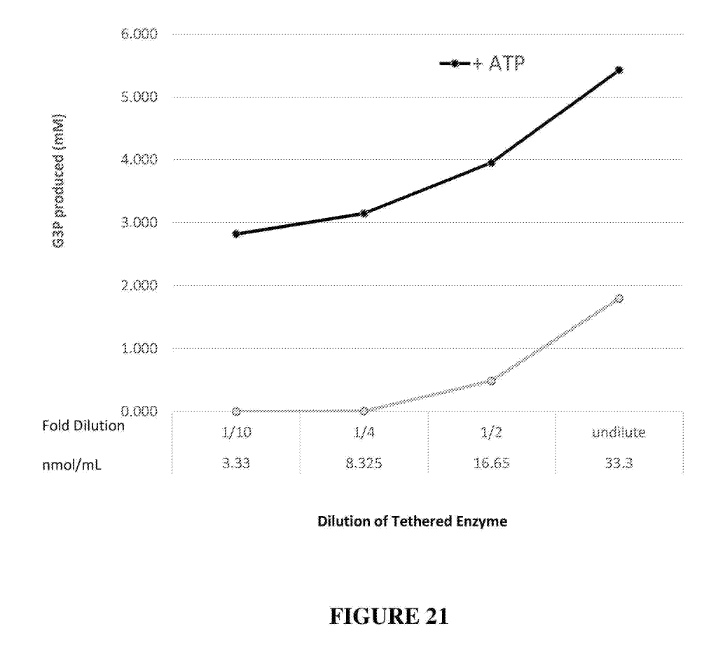

Tethered TriF1-PEG-ATP activity was titrated in the presence and absence of ATP to determine the efficiency of tethering. The tethered ATP without exogenous ATP had approximately 40% of the activity of enzyme with added ATP indicating incomplete tethering of modified cofactor to all fusion protein molecules (FIG. 21). Titration of diluted enzyme confirms that after two fold dilution, 20% of activity remains and after 4 fold dilution no tethered ATP activity remains suggesting that tethering was indeed ˜40% efficient.

Nonetheless the tethered enzyme biocatalyst was sufficiently active under batch reaction conditions that it could be coupled with TriF2 and aldolase enzyme to effectively produce as much fructose-1,6-biphosphate as similar coupled reactions with untethered TriF1 enzyme and added ATP.

Based on partially effective tethering above, the tethered cofactors were able to be turned over very effectively. Assuming 40% efficiency in an enzyme preparation of 33.3 μM (i.e. 13.32 μM ATP-PEG-TriF1, diluted 250 fold in the enzyme reaction to ˜50 nM), the tethered ATP molecules have been turned over ˜40,000 times to yield 2 mM glycerol-3-phosphate during the one hour incubation.

### Tethering NAD-2AE-PEG₂₄-MAL to TriF2

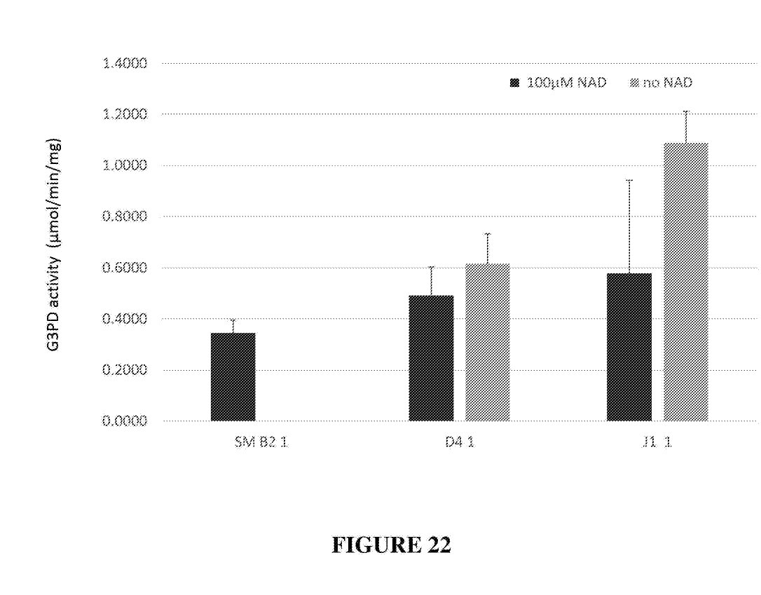

Having demonstrated successful tethering of modified NAD to the bienzymatic fusion protein BiF2, it was necessary to further confirm successful tethering of modified NAD to the trienzymatic fusion protein TriF2. Modified NAD with a polyethylene glycol tail was attached to a cysteine residue within the linker region of TriF2 using similar methods to those described above. Fusion protein was tethered to modified NAD with close to 100% efficiency and the resultant TriF2 nanomachine biocatalyst was able to successfully convert G3P to DHAP without the addition of exogenous NAD cofactor (FIG. 22), and could also be coupled with the aldolase enzyme ScFruA to produce several different chiral aldol sugars.

### Example 2—Flow Cell System Development

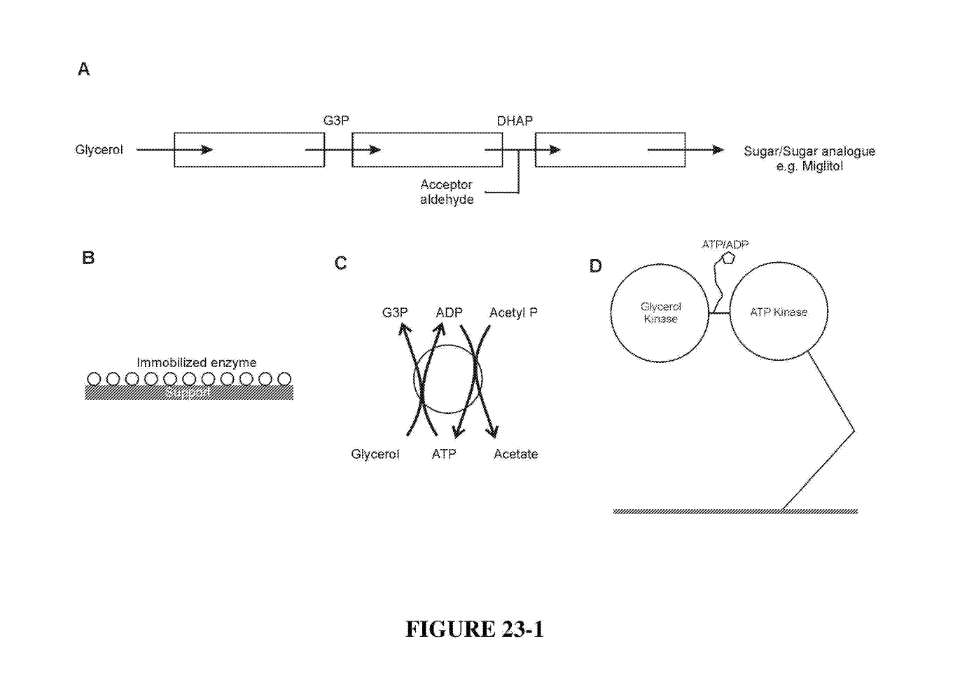

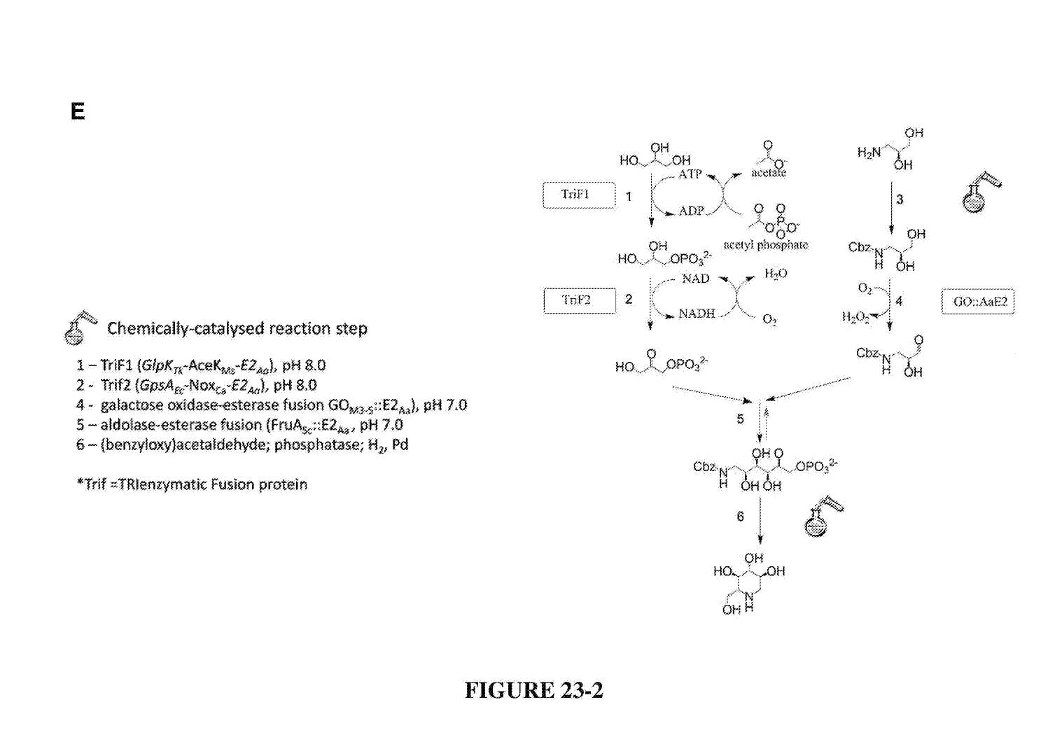

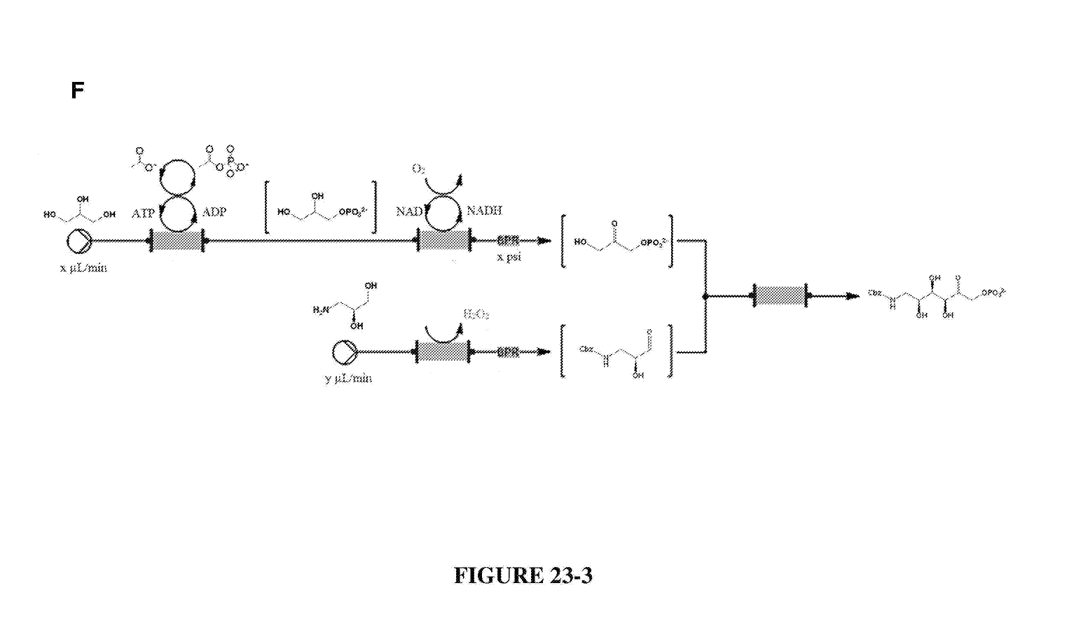

The development of a flow cell system requires tethering of the enzyme fusions to a solid support. An exemplary flow reactor concept is shown in (FIG. 23).

A simple model flow reactor was produced using agarose beads cross-linked to alcohol dehydrogenase enzyme, and demonstrated to function successfully. Flow rate was optimized at ˜0.7 mL per minute.

### Activation of Cotton

Since cotton, like agarose is a polysaccharide with primary hydroxyl groups available for chemical modification woven cotton was assessed for its ability to provide a fibre-based support for immobilised enzymes.

A solution of 25 mL 0.5 M Na₂CO₃ at pH 12 then 250 μl of Divinyl Sulfone (DVS) was added to 1 g of cotton discs (14 mm diameter). The suspension was then mixed for 60 min at room temperature. The DVS solution was poured from the cotton and 25 mL of water added and mixed to rinse. Rinsing was repeated 10 times (ranging from 2-20 minute incubations). The samples were then suspended in water overnight, drained then rinsed in 250 mL water for 30 minutes.

Conjugation of an Esterase Inhibitor to Cotton

To 5 μl of enzyme (CaNOX::AaF.2 or EcG3PDH:CaNOX::AaF2, TriF2) was added 1 μl of 0.2 M TFK inhibitor (1-bromo-3,3,3-trifluoroacetone and 1,1,1-trifluoro-3-(thiohexyl)propan-2-one) in DMSO and the solutions incubated for 5 min on ice before the residual esterase activity was determined. Esterase activity was determined from the hydrolysis of para-nitrophenylacetate, with monitoring at 405 nm.

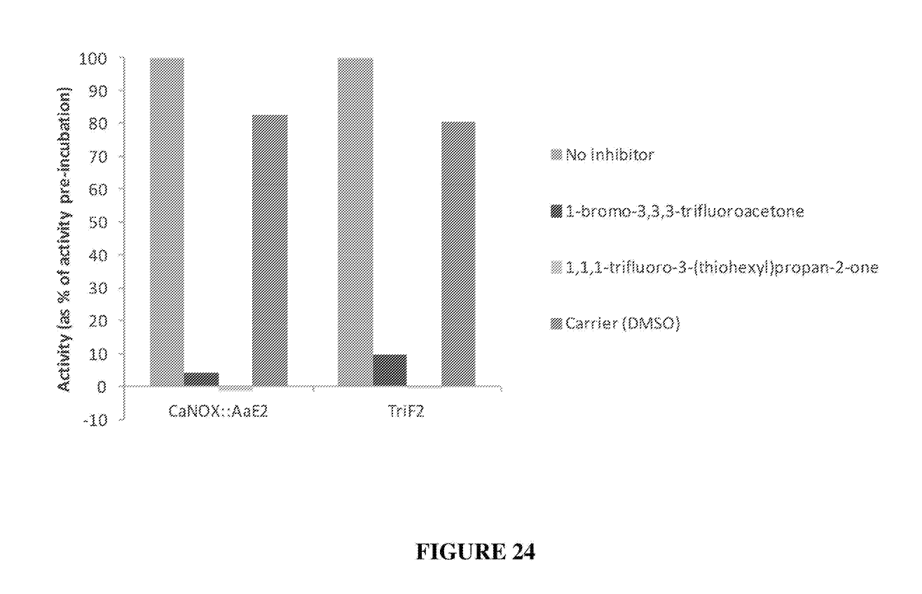

Esterase activity of the fusions CaNOX::AaE2 and EcG3PD::CaNOX::AaE2 (TriF2) was found to be greatly decreased (1-bromo-3,3,3-trifluoroacetone) or completely abolished (1,1,1-trifluoro-3-(thiohexyl)propan-2-one) after 5 min incubation with these esterase inhibitors (FIG. 24). These data indicated that the fusion proteins could be conjugated to a solid support using an esterase inhibitor.

### Production of Cotton-DVS-TFK Discs

After overnight soaking and washing, DVS-activated cotton was blotted to dryness. To the cotton was added 10 mL 0.1 M NaPi pH 8 and 10 mL 50% Ethanol. 200 μl of 0.1 M thiohexyl-TFA in DMSO was also added. The mixture was allowed to react on a rotating wheel for 4 hours. A 286 μl aliquot of 0.2 M 2-mercaptoethanol was added to the mixture and allowed to react overnight on a rotating wheel.

The cotton was washed with 50% ethanol for 10 washes, including blotting to dryness. The cotton was washed with water for 5 washes of 10 minutes, until the smell of DMSO was negligible. The samples were blotted to dryness and stored in a sealed bag at 4° C.

Immobilisation of ATP-CM-C₆-PEG₂₄-MAL-TriF1 to Cotton-DVS-TFK Discs

ATP-CM-C₆-PEG₂₄-MAL-TriF1 (12 mL, 20 mg, 150 nmol) was added to 1 g of cotton-DVS-TFK discs. After overnight incubation, the esterase activity in the supernatant had decreased from 11 U/mL to 2 U/mL, indicating about 80% of the esterase was immobilised to the support.

### Immobilisation of TriF2 to Cotton-DVS-TFK Discs

TriF2 was immobilised to the cotton-DVS-TFK discs directly. TriF2, purified by IMAC was further fractionated by gel filtration in PBS containing 0.1 mM TCEP. The material eluting at the expected volume for a dimer of the trifunctional fusion (the NOX enzyme forms a non-disulfide bonded homo-dimer) was pooled and 28 mL (0.3 mg/mL, 8.4 mg, 112 U esterase) was added to 1.6 g damp cotton-DVS-TFK discs (corresponding to 1 g dry cotton). The mixture was rotated on a wheel at 4° C. for 75 min before the supernatant was removed and the discs washed 4×50 mL PBS containing 0.1 mM TCEP. No activity was detected in the final wash.

The protein and esterase activity in the starting material and supernatant after immobilisation was determined (Table 12).

TABLE 12
Protein and esterase activity in the starting material and
supernatant after immobilisation.

| | Starting | After | Amount |
|---|---|---|---|
| | material | immobilisation | immobilised |
| A280 | 0.269 | 0.145 | |
| [protein] (mg/mL) | 0.33 | 0.18 | |
| Volume | 28 | 28 | |
| Protein (mg) | 9.12 | 4.92 | 4.20 |
| Esterase activity (U/mL) | 4.16 | 1.02 | |
| Esterase activity (U) | 116.48 | 28.56 | 87.92 |

### Preparation of Immobilised TriF2 Tethered to NAD-2AE-PEG₂₄-MAL

To half the discs (0.5 g dry cotton, 2.1 mg immobilised protein, 44 Units esterase) suspended in 10 mL buffer was added 1 equivalent NAD-2AE-PEG₂₄-MAL (based on the estimate of the amount of protein immobilised) (Batch B1). To the other half was added 10 equivalent NAD-2AE-PEG₂₄-MAL (Batch B2). The disc suspension was rotated on a wheel at 4° C. overnight.

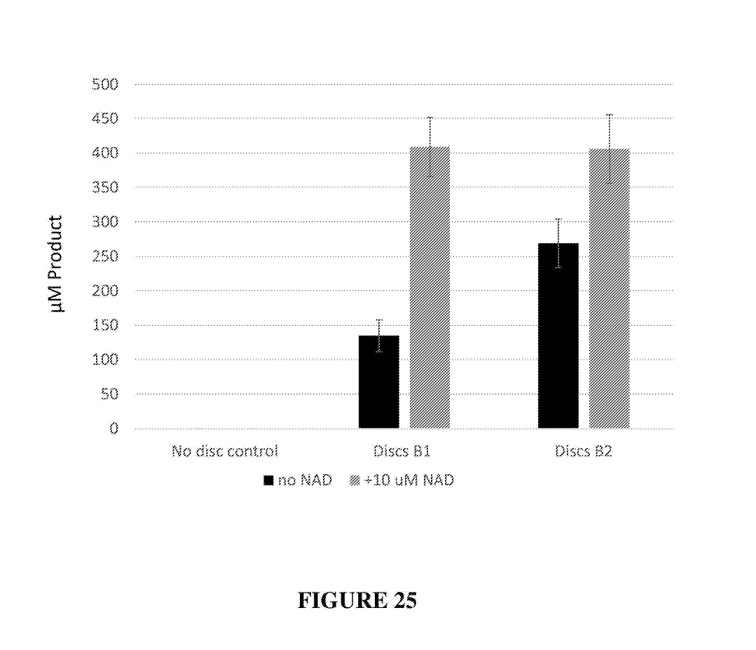

Conjugation of TriF2 onto TFK-treated cotton discs followed by the tethering of modified NAD was successful. Batch B2 was more active in the absence of added exogenous NAD+ than Batch B1 illustrating that increasing the molar equivalent of modified NAD+ used for tethering improved the efficiency of tethering (FIG. 25). Batch B2 discs reacted in the absence of exogenous NAD+ yielded ˜50% the DHAP production of fusion enzyme with added NAD+, suggesting tethering of ˜50% of the fusion proteins.

### Suitability of Cotton as a Material in a Bioreactor

It has been shown that cotton could be functionalized with a number of enzymes using different chemistries (Albayrak et al., 2002), (Edwards et al., 2011), (Kim et al., 2007).

Knitted cotton cloths were punched into discs of 11 mm in diameter. The discs were then packed tightly into a low pressure liquid chromatography (LC) column (Omnifit D=10 mm, L=100, bed volume=5.5 mL mm) into plugs of 15 or 30 mm in lengths. The diameter of the discs was selected to be larger than the inner diameter of the column to minimize channeling effect. The column was then connected to a Vapourtec flow reactor system equipped with sample injection loops and back pressure sensors.

Flow rates of 0.5 mL/min and 1.0 mL/min were used. Food dye was pumped through the columns for a period of 5 min and back pressures were monitored. It was found that for both packing lengths and flow rates, there were no back pressures, meaning the there was almost no resistance to the flow of reagents despite tight packing and long plug of discs (Cybulski and Moulijn, 2005). After the experiment, the discs were taken out and visually inspected. It was found that the dye was uniformly distributed across the disc surfaces and there was no channelling effect (Butt, 2000). These two findings suggest tightly packed cotton is a good candidate as support material for flow reactors.

The mean residence time and residence time distribution are two important parameters in the design process of reactors. The mean residence time should ideally be higher than the characteristic reaction time to avoid decomposition of the products and unwanted side reactions. This also helps to increase the yield of the reaction and reduce the reactor size. On the other hand, a narrow residence time distribution is preferred so that the times chemical species spend in a reactor are as close as possible, resulting in product homogeneity (Hessel et al., 2015).

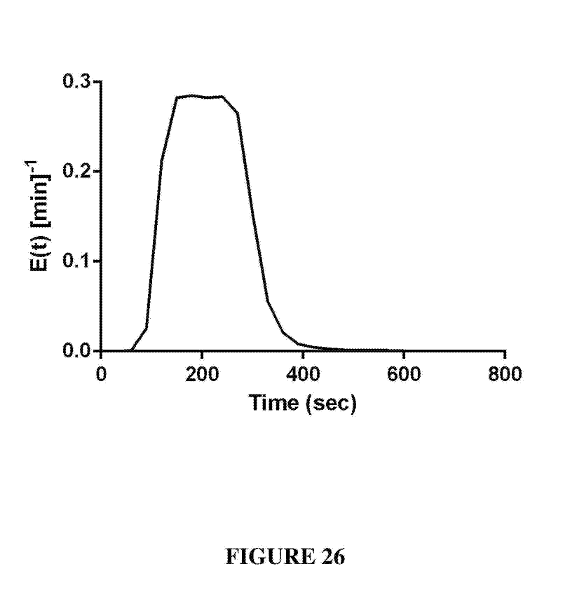

Residence time distribution (RTD) and mean residence time measurements were assessed in the reactor packed with 3 cm plug of cotton discs. A plug of 1 mL of food dye as a tracer was injected into the reactor running at 1 mL/min. Different dilutions of food dye were collected into 20 vials in every 30 sec. UV/VIS measurements were carried out at 632 nm on the vials to obtain the absorbance which can be converted into concentrations using Beer's Lambert law (FIG. 26). The mean residence time was calculated to be 6.7 min which appeared to be larger than the reaction characteristic time.

### TriF1 Flow Reactor (Step 1: Conversion of Glycerol to Glycerol-3-Phosphate)

Cotton discs with immobilised and tethered TriF1 were packed into an XK 16/20 column (GE Healthcare) with adaptors fitted to minimise the dead volume of the bioreactor.

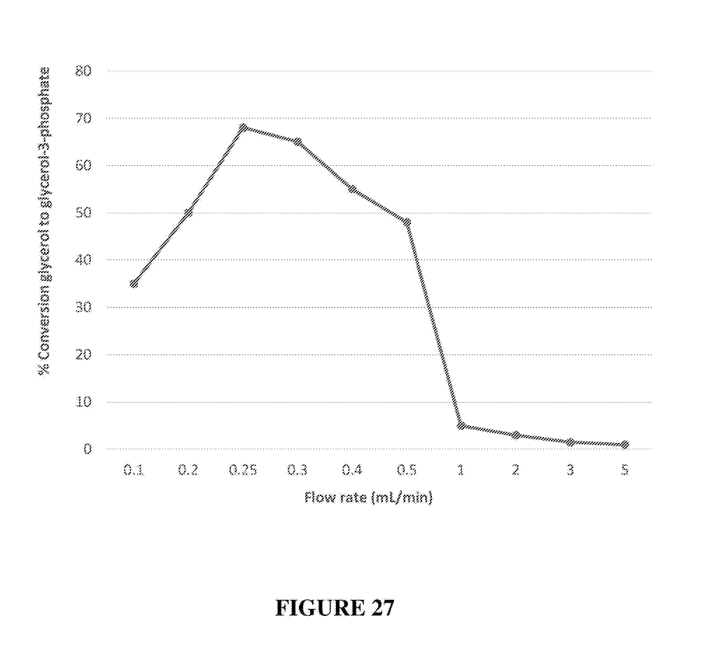

The flow rate was varied from 0.1 mL per minute to 5 mL per minute and the yield of glycerol-3-phosphate produced in each fraction assessed over time by LC-MS analysis (FIG. 27). Flow rate was optimal at 0.25 mL per minute and decreased substantially at flow rates of over 1 mL per minute.

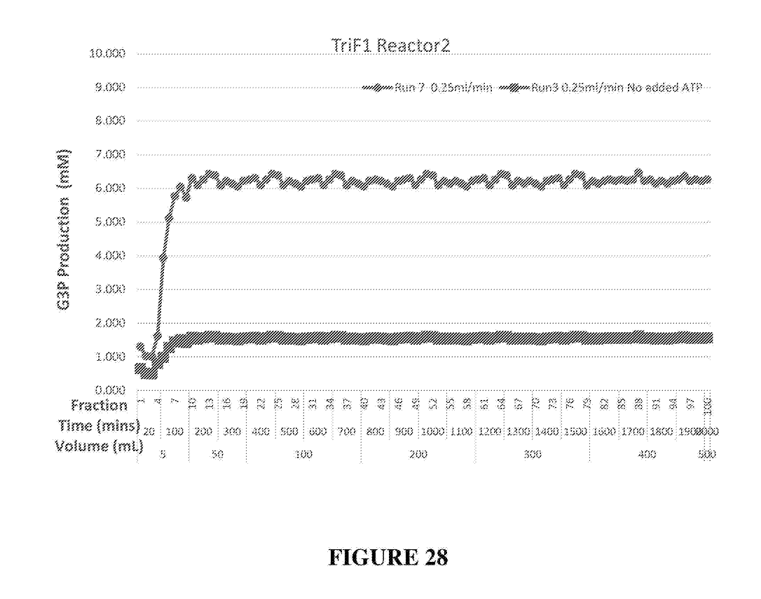

500 mL of reaction mixture containing 10 mM glycerol substrate was feed into T1R2 at 0.25 mL per minute for 33 hours, with 5 mL fractions collected over every 20 minutes. As illustrated in FIG. 28, the reactor reached maximum yield after ˜100 minutes (fraction 5) and operated steadily at maximum yield rate (˜60% conversion of glycerol to glycerol-3-phosphate) continuously for the remainder of the 33 hours.

Addition of a small amount of exogenous ATP to the reactor achieved maximum yields. However, it is worth noting that once the T1R2 flow reactor reached a steady state, the small amount of exogenous ATP added in Run 7 was continuously maintained a turnover number of 600 total turnovers per molecule for 33 hours.

### TriF2 Flow Reactor (Step 2: Conversion of Glycerol-3-Phosphate to DHAP)

Cotton discs with immobilised and tethered TriF2 were packed into an XK 16/20 column (GE Healthcare) with adaptors fitted to minimise the dead volume of the bioreactor.

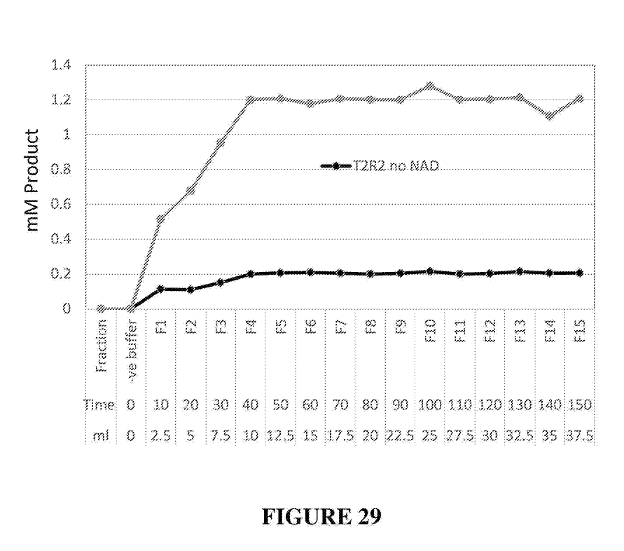

The NAD-tethered TriF2 flow reactor was capable of converting glycerol-3-phosphate to DHAP continuously for at least several hours, without the addition of exogenous NAD⁺ (FIG. 29).

### Immobilisation of Enzyme Fusion TriF2 Containing the Esterase Module to Esterase Inhibitor Covalently Attached to a Solid Support

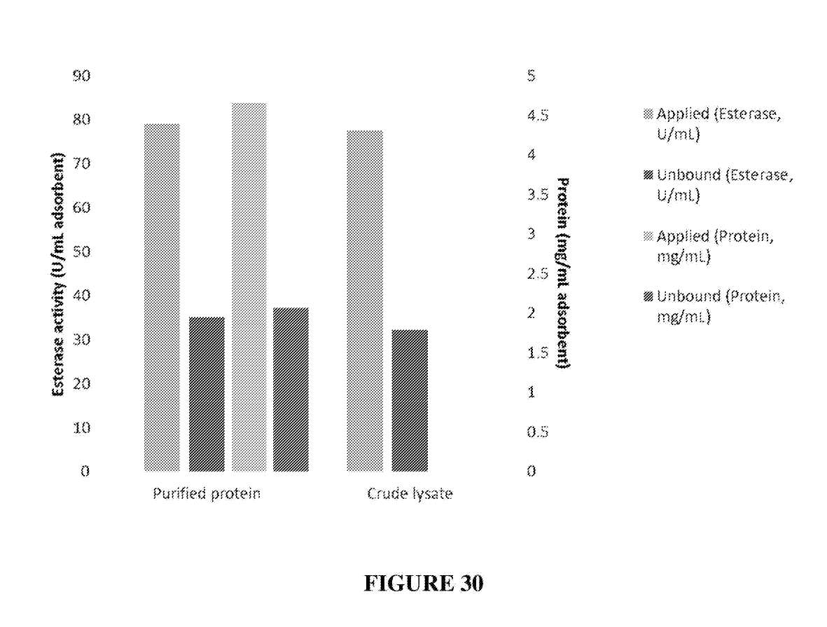

TriF2 purified on a HisTrap column (5 mL) followed by gel filtration on a Superdex 200 2660 column was immobilised to the Sepharose-vinylsulfone-thiohexyltrifluoroketone beads (2.5 mg per mL beads). Alternatively crude lysate containing TriF2 was applied directly to the Sepharose-vinylsulfone-thiohexyltrifluoroketone beads with approximately 45 units of esterase activity binding per mL beads (which equates to a very similar capacity to that observed for the purified protein (FIG. 30)

### Tethering of Maleimide-PEG₂₄-2AE-NAD to TriF2 Immobilised or in Solution

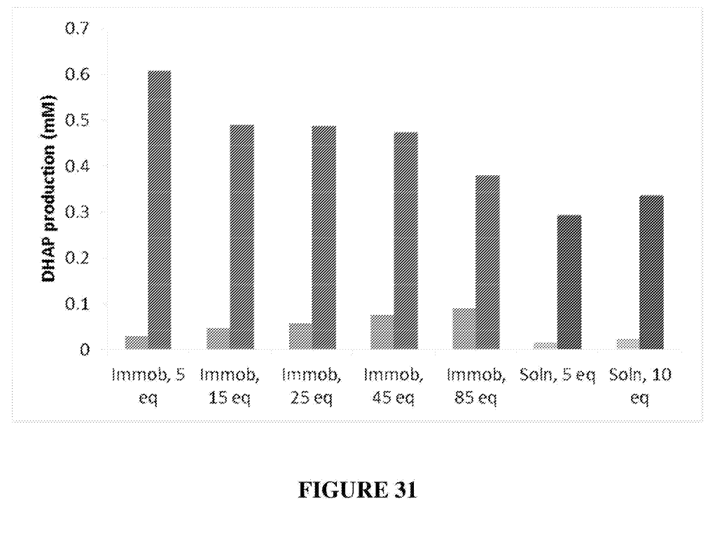

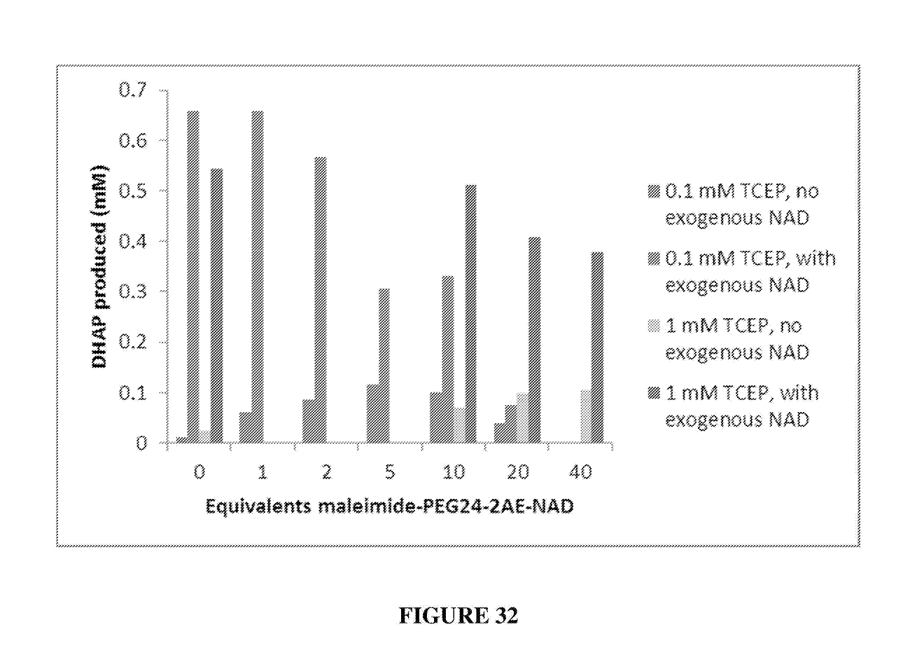

Purified TriF2 was reacted with 5 or 10 molar equivalents of maleimide-PEG₂₄-2AE-NAD for 1 hour at 4° C. in the presence of 1 mM TCEP. The reaction mixture was directly immobilised to Sepharose-TFK beads and unbound protein and cofactor removed by washing before the DHAP production was assayed in the presence and absence of exogenous NAD. In an alternative approach the TriF2 was immobilised directly from crude lysate and the amount of protein immobilised estimated from the loss of esterase activity in the unbound fraction. This TriF2 was reacted with from 5-85 molar equivalents of the maleimide-PEG₂₄-2AE-NAD for 1 h in the presence of 1 mM TCEP before unbound cofactor was removed by washing and the DHAP production assayed. Cofactor was successfully tethered by both methods, as judged by the ability to produce DHAP in the absence of exogenous NAD(H) (FIG. 31).

### Optimisation of Tethering of Maleimide-PEG₂₄-2AE-NAD to Immobilised TriF2

Immobilised TriF2 was reacted with maleimide-PEG₂₄-2AE-NAD (0-40 equivalents) in the presence of 0.1 mM or 1 mM TCEP for 1 h at 4° C. before being washed to remove unbound cofactor and assayed for DHAP production in the presence of absence of exogenous NAD(H). At higher concentrations of cofactor there was loss of TriF2 activity, especially at 0.1 mM TCEP, while at lower concentrations there was very little tethering (as judged from the lack of DHAP production in the absence of exogenous cofactor).

### Example 3—Nanofactory Comprising Three Nanomachine Flow Reactors

### Preparation of Sepharose Beads with Immobilised 1,1,1-trifluoro-3-((6-mercaptohexyl)thio)propan-2-one (TFK)

To a slurry of vinylsulfone-activated agarose (800 mL, 600-800 mmol of vinyl sulfone groups, 50% slurry in 1:1 ethanol/water) was added saturated aqueous NaHCO₃ solution (80 mL), 1,1,1-trifluoro-3-((6-mercaptohexyl)thio)propan-2-one (104 mg, 0.4 mmol) dissolved in ethanol (4.8 mL). The mixture was stirred gently at room temperature overnight. The excess reactive sites were blocked by the addition of 2-mercapto ethanol (11.2 mL, 80 mmol) followed by continued stirring for 6 h. The resin was then washed extensively with 50% ethanol/water until no smell was evident. Beads were stored as 1:1 slurry in 50% ethanol/water.

### Triple Multi-Enzyme Reactor Using Fusion Enzymes Immobilised on TFK-Derivatised Sepharose Beads

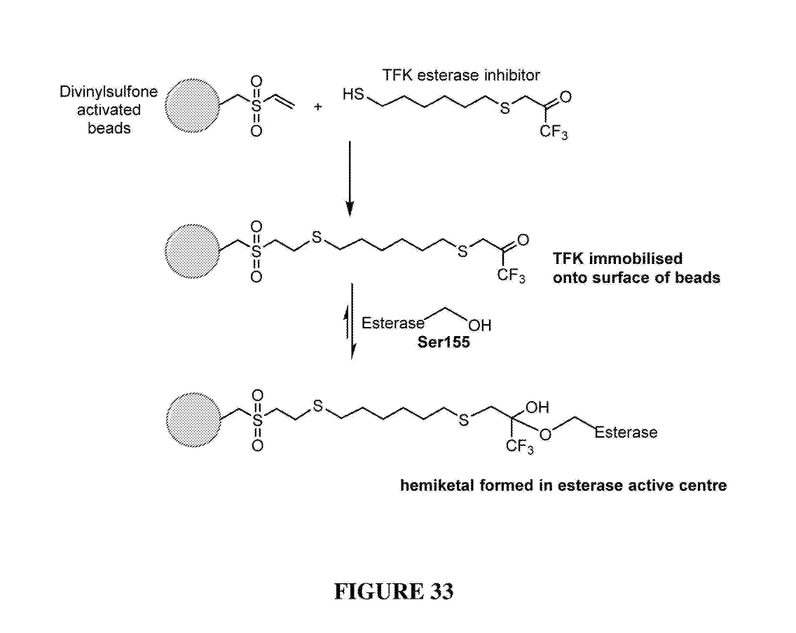

TriF2 (EcG3PD-CaNOX-AaE2) with tethered mNAD, galactose oxidase _{M3-5}-esterase AaE2 and ScFruA aldolase-esterase fusion proteins were immobilised onto hexyl-TFK derivatised beads through covalent bonding between the esterase component of the fusion enzymes and the ketide group of TFK (FIG. 33). Immobilised enzyme bead activity was assessed as shown in Table 13.

TABLE 13
Specific activity of fusion-enzymes immobilised on
TFK-derivatised beads.

| | Enzyme Activity | | Specific |
|---|---|---|---|
| Fusion Enzyme | (nmol per μL | Protein Conc. | Activity |
| Nanomachine | beads/min) | (mg/mL beads) | U/mg protein |
| mNAD-tethered TriF2 | 0.25 ± 0.08 | 1.34 ± 0.07 | 0.19 ± 0.01 |
| Galactose oxidase | 34.5 ± 2 | 0.368 ± 0.02 | 93.7 ± 0.3 |
| M₃₋₅-esterase | | | |
| Aldolase | 1.23 ± 0.3 | 0.198 ± 0.01 | 5.99 ± 1.3 |
| ScFruA-esterase | | | |

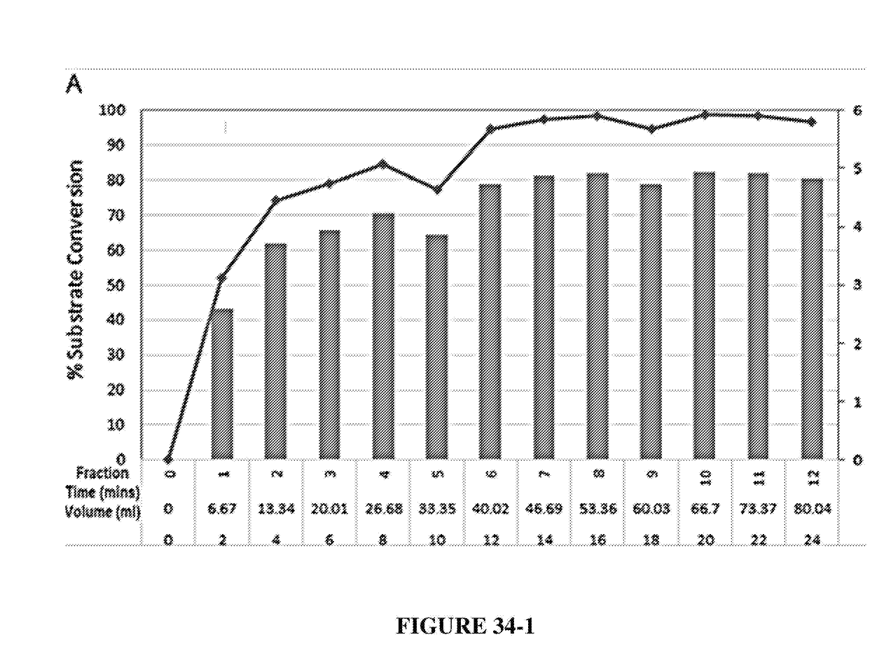

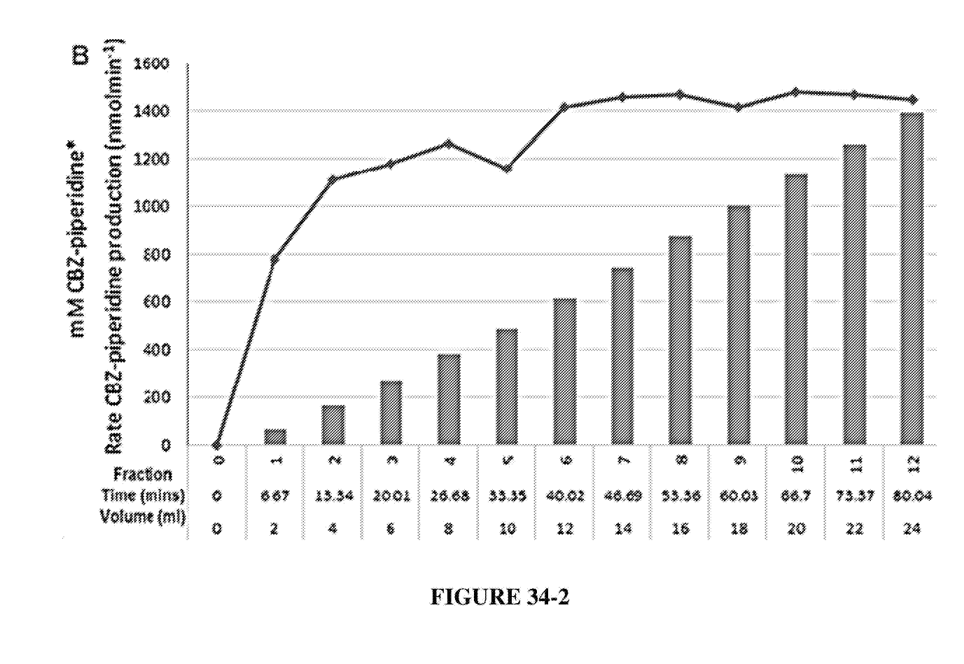

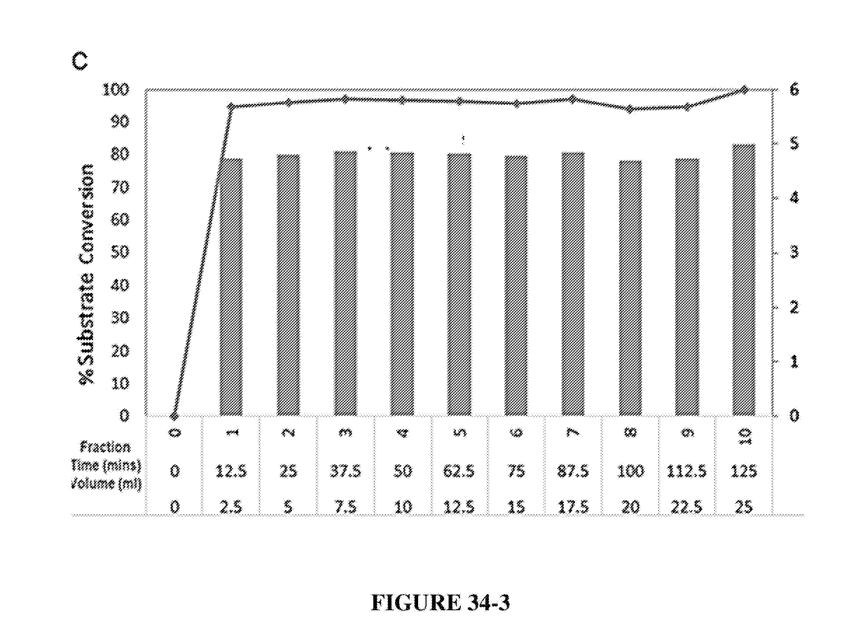

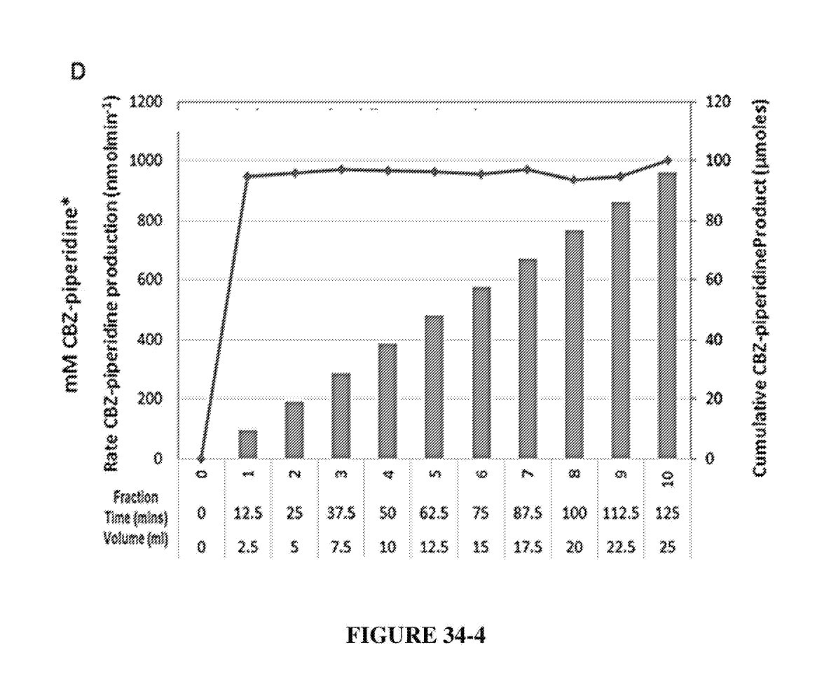

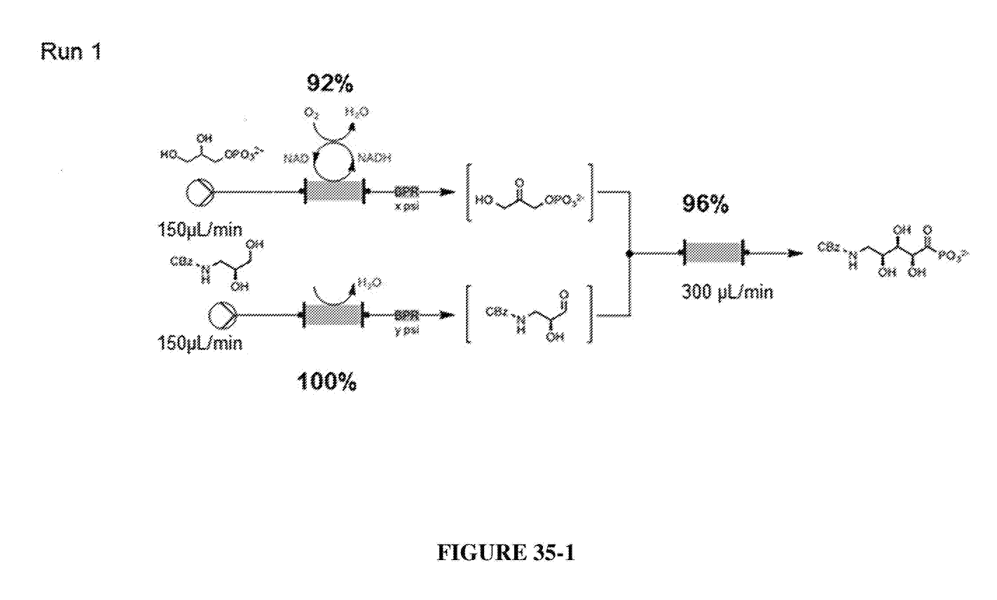

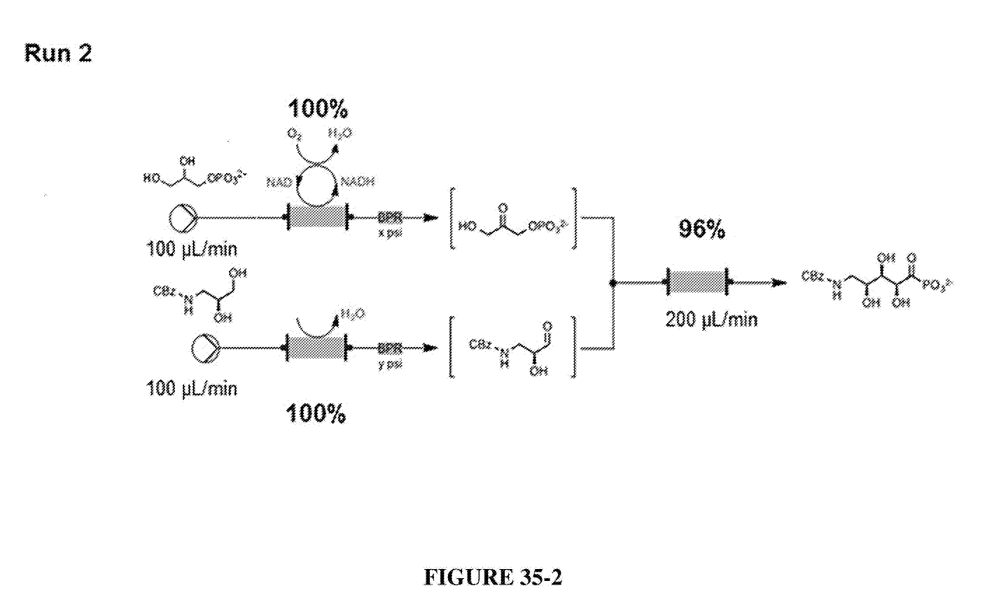

One separate Omniflow column was packed with estimated sufficient slurry to fully convert 5 mM substrate for each of the immobilised fusion enzyme beads. Each nanomachine enzyme flow reactor was then assessed individually, before combining the nanomachine flow reactors into a three part multi-enzyme nanomachine flow reactor (nanofactory) which yielded up to 96% conversion of 5 mM glycerol-3-phosphate and 5 mM CBZ-aminopropanediol into the CBZ protected amino ketohexose phosphate (FIG. 34 and FIG. 35).

These data demonstrate successful conversion of CBZ-protected aminopropanediol into the Miglitol precursor molecule (denoted CBZ protected amino ketohexose phosphate) using a triple multi-enzyme flow reactor (nanofactory) comprising three nanomachine flow reactors with fusion enzymes immobilised on beads. This multi-enzyme cascade reactor yielded 96% conversion of substrate into product (FIG. 35).

### Example 3—Extension of Nanomachine Concept

The nanomachine biocatalyst system concept can be extended to encompass a number of other industrially relevant reaction chemistries catalysed by enzymes that require nicotinamide cofactors. Table 14 demonstrates functional bienzymatic fusion proteins for three other chemistries: Enoane reduction, chiral amine synthesis and production of chiral secondary alcohols.

The functionality of the purified bi-enzymatic fusion proteins BiF5, 6, and 7 was assessed (Table 15). BiF5 was shown to be able to produce R-levodione from keto-isophorone, and also to efficiently recycle NADPH to NADP⁺ via reduction of ethanol to acetaldehyde. The added NADPH cofactor was turned over a total of 358 times within that hour by the fusion protein. BiF6 demonstrated both efficient recycling of NADH to NAD⁺ and production of S-octanol from octanone, with nearly one hundred percent conversion of 7.7 mM substrate within one hour. BiF7 was purified and shown to be able to produce enantiomerically-pure branched chain and aromatic D-amino acids from ketoacid substrates.

TABLE 15
Efficiency of bi-enzymatic fusion proteins BiF5, BiF6, BiF7 for enoane
reduction of ketoisophorone, production of chiral secondary alcohols and production of
chiral amines (respectively).

| | Component enzymes | | | Rate | | |
|---|---|---|---|---|---|---|
| Bienzymatic | | Cofactor- | % Total | Product | Enantiomeric | |
| Fusion | Synthetic | Recycling | Conversion | formation | Excess | TTN |
| Protein (BiF) | Component | Component | of substrate | (μM⁻¹s⁻¹) | (EE; %) | (min⁻¹) |
| BiF5 | SYE2 | GtADH | 43% | 4.83 ± 0.09 | 99.9% | 35.8 ±3.7 |
| | | | (±2.5%) | (R- | | (NADPH) |
| | | | | levodione) | | |
| BiF6 | GtADH | BacFDH | 63.9% | 20.1 ±1.23 | 99.5% | 72.3 ±3.7 |
| | | | (±8.2%) | (S-octanol) | | (NADH) |
| BiF7 | UtDAADH | BacFDH | 87.9% | 6.0 ±0.56 | 98.9% | 36.1 ±2.1 |
| | | | (±4.5%) | (D- | | (NADPH) |
| | | | | leucine) | | |
| | | | 35.4% | 9.08 ±3.42 | 99.6% | 22.71 ± 1.7 |
| | | | (±3.6%) | (D- | | (NADPH) |
| | | | | tyrosine) | | |

Reactions were conducted at room temperature in 1 mL total volume with 5-50 mM starting substrate, between 1 and 14 nM of enzyme and 100 μM each of NADH or NAD(P)H as required. Samples were collected after 1 hour and analysed by LCMS, chiral HPLC or chiral GC as described in methods. TTN—total turnover number (min⁻¹).

### Example 4—Biocatalyic Flow Reactors

### D-Fagomine Nanofactory

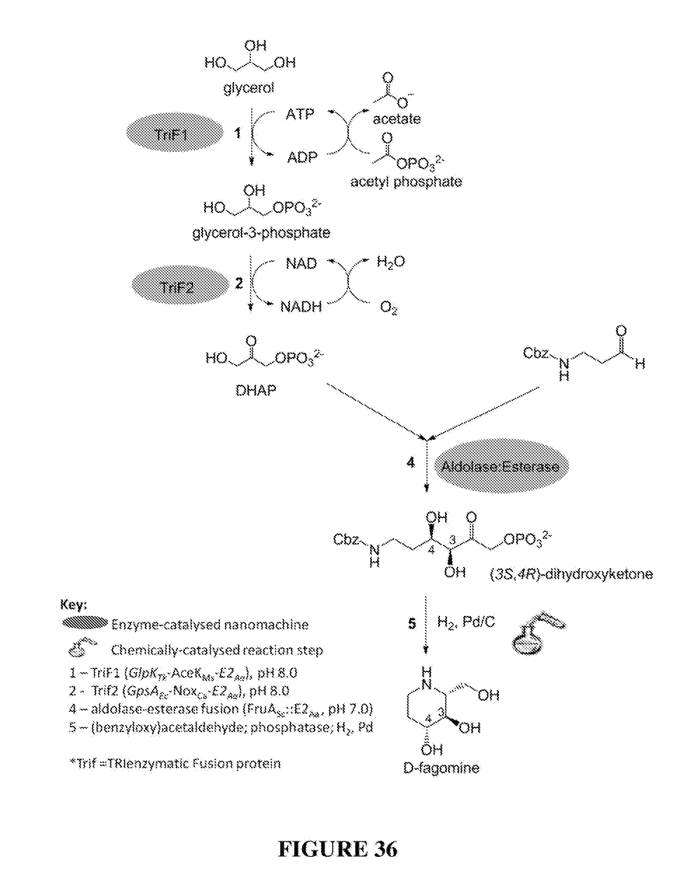

The functionality of the immobilised nanomachine in reactors which both retain and recycle cofactors for flow biocatalysis was demonstrated via production of D-fagomine, an important commercially relevant anti-diabetic drug. D-fagomine can be produced enzymatically from glycerol via two regiospecific, cofactor-dependent steps (an ATP-dependent phosphorylation and an NAD-dependent oxidation) and a stereospecific aldol condensation), followed by chemical cyclisation (FIG. 36).

### The Phosphotransfer Reactor

For the preparation of the TriF1 phosphotransfer reactor (step 1 in FIG. 36), 40 milligrams of TriF1 protein (296 nmoles) was immobilised onto 25 g of sepharose-hexyl-DVS-TFK beads. The immobilised TriF1 was treated with TCEP, washed with degassed, sparged PBS containing 0.5 mM EDTA then reacted with six equivalents ADP-2AE-PEG₂₄-NAD for 6 h at 4° C. before being washed with PBS. The resultant nanomachine beads were analysed for glycerol kinase activity in the presence and absence of ATP in batch reactions, and demonstrated to have ˜10% tethering efficiency. The resultant nanomachine beads comprising immobilised ADP-2AE-PEG₂₄-TRIF1 were then packed into a 25 mm*15 mm Benchmark column (Kinesis, Australia) and assessed in a flow reactor system.

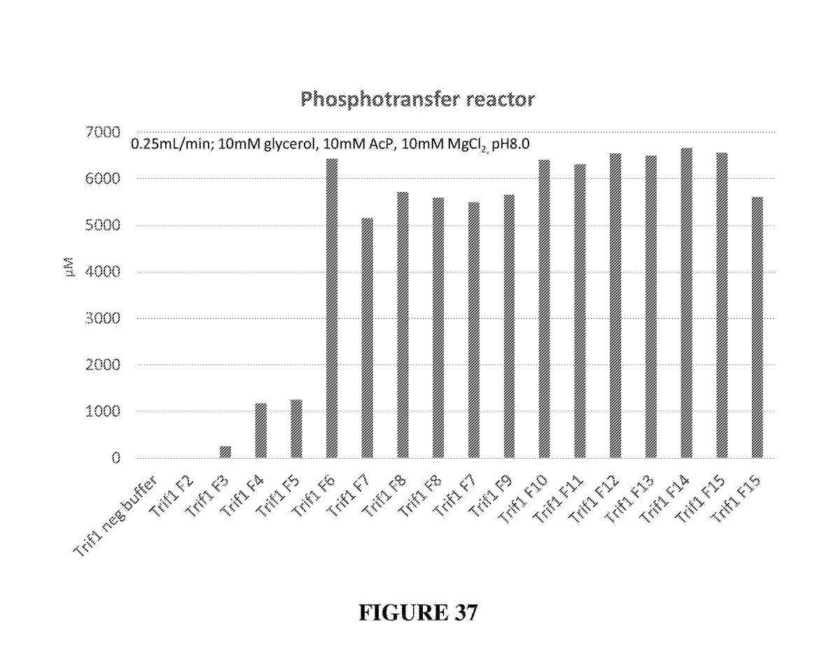

A bioreactor packed with the nanomachine beads comprising immobilised ADP-2AE-PEG₂₄-TRIF1 was found to convert 10 mM glycerol and 10 mM acetyl phosphate to G3P and acetate with approximately 60% efficiency at the optimal flow rate of 0.25 mL/min (FIG. 37). This resulted in a space time yield of 70 mg G3P L⁻¹hr⁻¹ mg⁻¹ protein. The bioreactor stability was further assessed by continuing to run the phosphotransfer reactor for a total time of 870 minutes resulting in a total 14222 turnovers of the tethered cofactor. The phosphotransfer reactor

For the preparation of the TriF1 phosphotransfer reactor (step 1 in FIG. 36), milligrams of TriF1 protein (296 nmoles) was immobilised onto 25 g of sepharose-hexyl-DVS-TFK beads. The immobilised TriF1 was treated with TCEP, washed with degassed, sparged PBS containing 0.5 mM EDTA then reacted with six equivalents ADP-2AE-PEG₂₄-NAD for 6 h at 4° C. before being washed with PBS. The resultant nanomachine beads were analysed for glycerol kinase activity in the presence and absence of ATP in batch reactions, and demonstrated to have ˜10% tethering efficiency. The resultant nanomachine beads comprising immobilised ADP-2AE-PEG₂₄-TRIF1 were then packed into a 25 mm*15 mm Benchmark column (Kinesis, Australia) and assessed in a flow reactor system.

A bioreactor packed with the nanomachine beads comprising immobilised ADP-2AE-PEG₂₄-TRIF1 was found to convert 10 mM glycerol and 10 mM acetyl phosphate to G3P and acetate with approximately 60% efficiency at the optimal flow rate of 0.25 mL/min (FIG. 37). This resulted in a space time yield of 70 mg G3P L⁻¹hr⁻¹mg⁻¹ protein. The bioreactor stability was further assessed by continuing to run the phosphotransfer reactor for a total time of 870 minutes resulting in a total 14222 turnovers of the tethered cofactor.

### The Oxidation Reactor

For the preparation of the TriF2 oxidation reactor (step 2 in FIG. 36), 80 milligrams of TriF2 protein (647 nmoles; 1260 esterase U) was immobilised onto 80 g of sepharose-hexyl-DVS-TFK beads. The immobilised TriF2 was treated with TCEP, washed with degassed, sparged PBS containing 0.5 mM EDTA then reacted with six equivalents ADP-2AE-PEG₂₄-NAD for 6 h at 4° C. before being washed with PBS. The resultant immobilised cofactor-tethered nanomachine beads were analysed for glycerol-3-phosphate dehydrogenase activity in the presence and absence of NAD+ in batch reactions, and demonstrated to have ˜80% tethering efficiency. The resultant nanomachine beads comprising immobilised ADP-2AE-PEG₂₄-TRIF2 were then packed into a 250 mm*15 mm Benchmark column (Kinesis, Australia) and assessed in a flow reactor system.

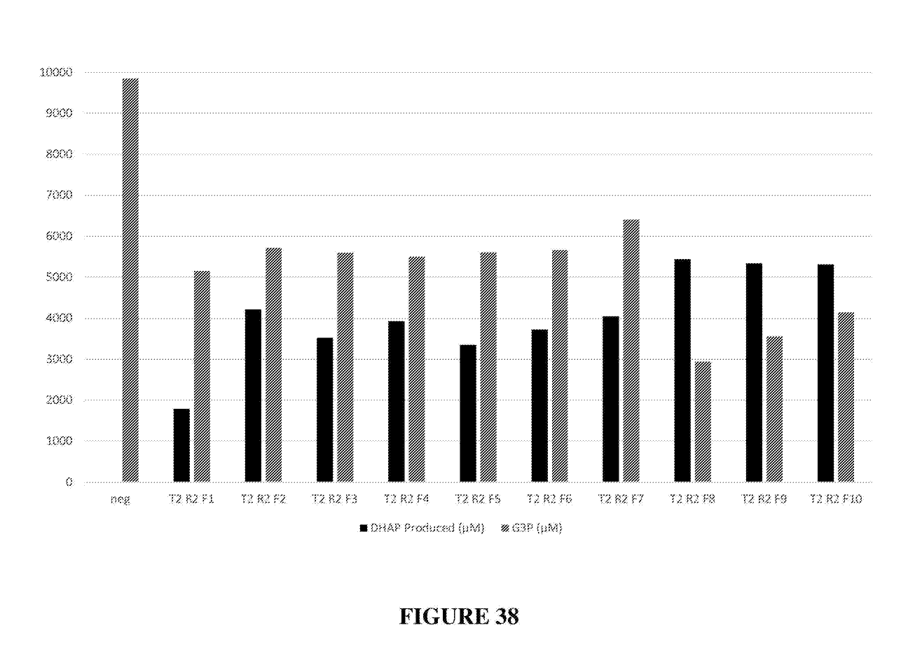

The column packed with the nanomachine beads was found to convert 10 mM G3P to DHAP with about 40-50% efficiency at a flow rate of 0.25 mL/min (FIG. 38).

### The Aldol Condensation Reactor

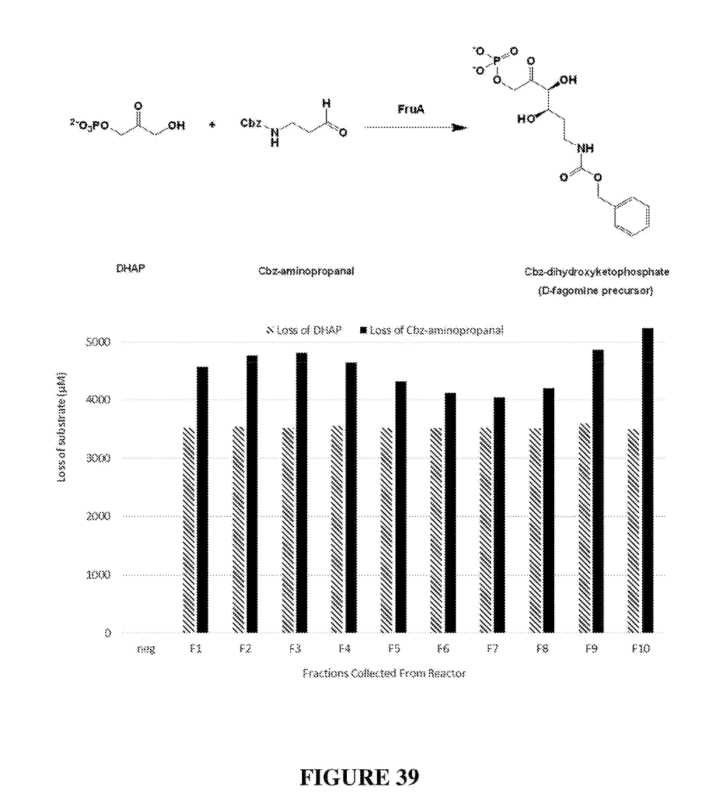

The binding of BiF4 (Staphylococcus carnosus aldolase (ScFruA)-Alicyclobacillus acidophilus esterase 2 (AAE2)) to Sepharose-DVS-hexyl-TFK beads was assessed using different ratios of enzyme to beads. Ratios of 0.5, 1 and 2 to one had no significant impact on activity per volume of immobilised beads, but a ratio of 0.5 to 1 was selected as optimal, as this ratio demonstrated the least loss of activity per mg of protein i.e. protein binding was already saturated at this ratio (FIG. 39).

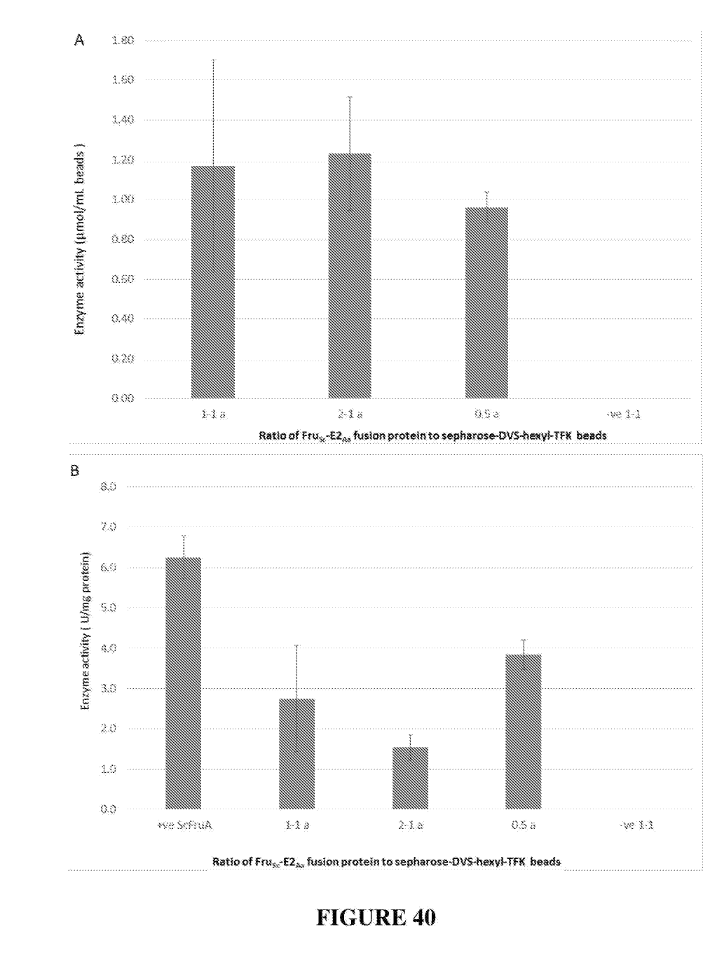

Using the optimised immobilisation conditions, 20 mg of BiF4 protein was reacted with 20 g of sepharose-hexyl-DVS-TFK beads. The resultant immobilised aldolase nanomachine beads were then packed into a 150 mm*15 mm Benchmark column (Kinesis, Australia) to a final length of 10 cm (17.7 mL packed bead volume) and assessed in a flow reactor system. Optimal flow rate was assessed for the aldol reactor and found to be 0.1 mL/min, with approximately 86% and 98% conversion of 5 mM Cbz-aminopropanal and 5 mM DHAP under these conditions (FIG. 40).

This resulted in a putative space time yield of 28.48 mg Cbz-dihydroxyketophosphate product L⁻¹hr⁻¹mg⁻¹ protein (noting that this is based on loss of substrate and not actual quantification of product) for the aldol condensation reactor under these conditions. The bioreactor stability was further confirmed by continuing to run the aldol condensation reactor for a total time of 840 minutes.

### Production of Aminocyclitol Via Serial Enzymatic Reactors

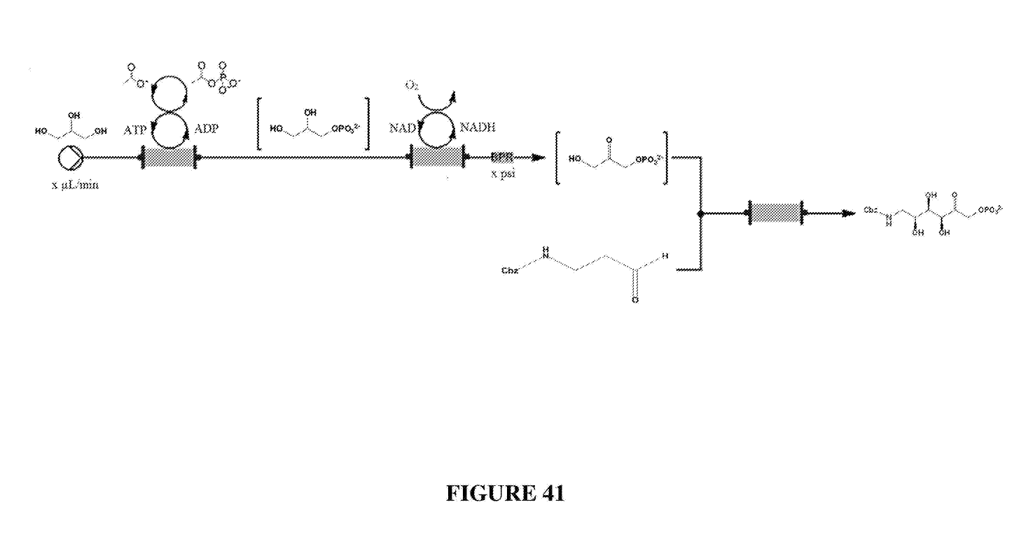

In order to demonstrate the combinatorial use of modular, hierarchical nanomachines to produce a commercially relevant fine chemical, the phosphotransfer, oxidation and aldol condensation reactors described above were combined to convert glycerol and Cbz-aminopropanal into the precursor for D-fagomine, a commercially relevant anti-diabetic drug as illustrated in FIG. 41.

The reactors were fed with 5 mM glycerol in 50 mM citrate buffer pH8.0 with 50 μM TCEP and systematically coupled together sequentially e.g. phosphotransfer reactor was run for at 0.25 mL/min for 40 mins, before adding the oxidation reactor in series at 0.25 ml/min and running both for 200 minutes, then including 5 mM Cbz-aminopropanal in 50 mM citrate pH 7.0 by a parallel pumping system and adding the aldol condensation reactor in series after this. The multienzyme reactor cascade was then run at 0.25 mL/min in this configuration for 1200 minutes (total volume 300 mL, hrs) and the fractions analysed for loss of substrate and detection of products over time.

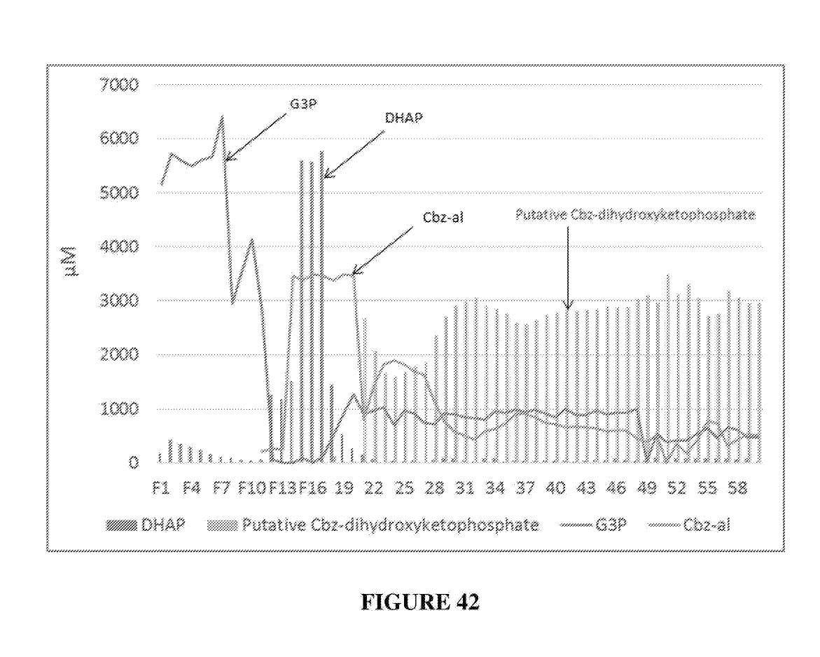

Analysis of the fractions collected during the operation of the serial reactor, demonstrates that the phosphotransfer reactor initially converted glycerol into glycerol-3-phosphate (F1-F7), then the sequential inclusion of the oxidation reactor resulted in conversion of the glycerol-3-phosphate into DHAP (F12-F17). The inclusion of the parallel pump feeding 5 mM Cbz-aminopropanal results in the appearance of this in F15-21 before the inclusion of the third and final aldol condensation reactor results in the loss of both glycerol-3-phopshate and DHAP, and the loss of the Cbz-aminopropanal substrate. The expected Cbz-dihydroxyketophosphate product was detected in fractions F18-60, but could not be accurately quantified due to the lack of a known standard for calibration curve. Thus the putative yield derived from loss of the Cbz-aminopropanal substrate as been illustrated in FIG. 42, but the exact yield will require confirmation with a known amount of a standard Cbz-dihydroxyketophosphate.

From the data it can be seen that the three reactors were not in perfect molar balance (Table 16) in this experiment, as there is some excess glycerol-3-phosphate and Cbz-aminopropanal produced. However, finer correction of the flow rates to balance the reactors using a more sophisticated flow reactor system should enable complete conversion of all starting glycerol substrate into the D-fagomine precursor.

Overall the metrics of the serial reactors for the production of the aminocyclitol precursor are very promising, with space time yields between 10 and 70 mg L⁻¹hr⁻¹ mg⁻¹ protein for each of the component reactors, and total turnover numbers for the tethered cofactors in the range of 104, making this system a viable demonstration of the production of a commercially relevant fine chemical.

TABLE 16
Summary of the serial reactor overall performance
characteristics for the biocatalytic continuous flow reactors.

| | | | | Total | Space Time |
|---|---|---|---|---|---|
| | | | Total | Turnover | Yield |
| | Flow rate | Rₜ | nMoles | Number | (mg L⁻¹ |
| Nanomachine | (mL/min) | (min) | Product | (cofactor) | hr⁻¹mg⁻¹) |
| Phosphoreactor | 0.25 | 84.8 | 1170997 | 16848 | 69.95 |
| TriF1 | | | | | |
| Oxidation | 0.25 | 113.2 | 953301 | 10839 | 10.75 |
| Reactor | | | | | |
| TriF2 | | | | | |
| Aldol | 0.1 | 177 | 4670395 | na | 28.58 |
| Condensation | | | | | |
| Reactor | | | | | |
| BiF4 | | | | | |

### Example 5—Materials and Methods

### Cloning, Expression and Purification of Enzymes

With two exceptions, enzymes were obtained by cloning, expression and purification from E. coli cells. Briefly, synthetic genes were transferred into either pDEST17 or pETCC2, transformed into E. coli BL21AI or E. coli BL21DE3* (Invitrogen) cells respectively. Cells were then induced for 2, 4, 6 or 24 hours with either 0.2M arabinose or 1 mM IPTG (respectively) and then harvested, resuspended in one tenth volume and lysed with Bugbuster (Novagen). Protein expression was analysed by SDS-PAGE separation stained with NuBlue (Novagen). The optimal expression time was selected and large scale expression cultures of 1-2 L prepared in the same way as above, followed by purification of HIS-tagged protein by elution with increasing concentration of imidazole from NiNTA column. If necessary the desired protein fractions were further purified using a GE 200 size exclusion column for elution. Pooled fractions were then concentrated and stored at 4° C., or −80° C. as required.

### Enzymic Activity Assays

Glycerol kinase assays were performed at room temperature in 1 mL volume essentially as described by (Pettigrew 2009), but with direct detection of ADP and ATP by HPLC analysis of reaction supernatant. A typical reaction contained 1 mM glycerol, 10 mM MgCl2, 50 mM NaHCO3 buffer pH 9.0, 1 mM ATP with approximately 2 μg/mL enzyme (35 nM). Kinetics were determined by varying the concentrations of ATP or glycerol whilst maintaining the other in excess, and kinetic determinants calculated using Hyper (J. S. Easterby, Liverpool University). Substrate and cofactor concentrations ranged from 0.1 to 10×Km.

Acetate kinase assays were conducted in the same manner, replacing ATP with ADP and glycerol with acetyl phosphate or phosphoenol pyruvate. Kinetics were determined by varying the concentrations of ADP or acetyl phosphate or phosphoenol pyruvate whilst maintaining the other components in excess, and kinetic determinants calculated using Hyper (J. S. Easterby, Liverpool University). Substrate and cofactor concentrations ranged from 0.1 to 10×Km.

Glycerol-3-phosphate dehydrogenase assays were conducted essentially as described by (Sakasegawa et al., 2004). Kinetics were determined by varying the concentrations of NAD/NADP or glycerol-3-phosphate, whilst maintaining the other components in excess, and kinetic determinants were calculated using Hyper (J. S. Easterby, Liverpool University). Substrate and cofactor concentrations ranged from 0.1 to 10×Km.

### LCMS Analysis of Ketones and Alcohols.

Octanone and octanol were separated using a modification of the method described in (Prieto-Blanc et al., 2010). Chromatographic conditions were SIELC ObeliscN column (250 mm) with 50% mobile phase A, 50% mobile phase B for 30 minutes. Mobile phase A: 20% ammonium formate pH 4.0; mobile phase b: acetonitrile. Mass spectrophotometric detection was conducted using API-ES mode (positive or negative as required) with an Agilent 6120 Quadropole LCMS. Compounds were quantified by selected ion monitoring of 113.19-m/z (heptanone) and 115.20-m/z (heptanol). R- and S-enantiomers of octanol were separated by chiral HPLC using 250 mm Chirobiotic column (Sigma-Aldrich), 1 mL/min with mobile phase methanol:water:triethylamine (25:65:10). Retention times at a flow rate of 1 mL/min were 3.73 min (S—) and 4.20 min (R—).

### Chiral GC Analysis of (R)- and (S)-Enantiomers of Octanol and Heptanol

Enantiomers were separated and detected after extraction into hexane. Chiral GC separation was performed with Chiraldex Astec ATA column (Sigma-Aldrich) using the following program on Agilent GC. 1 mL/min He at 100° C., hold for 0.2 min then ramp at 10° C./min to 250° C. and hold for 10 min. Injector temperature: 280° C. 1 μL sample was injected and products were detected by FID

### HPLC Separation of ATP and ADP

HPLC separation was conducted using an Agilent Eclipse XDB column (50 mm) with an isocratic gradient of 25% solvent A and 75% solvent B. Solvent A: acetonitrile; solvent B: 20 mM tetrabutylammonium phosphate (TBAP) in 10 mM ammonium phosphate buffer.

### LCMS Analysis of Glycerol-3-Phosphate (G3P), DHAP and Aldol Condensation Products

G3P and DHAP were separated using a modification of the method described in Prieto-Blanc et al., (2010). Chromatographic conditions were SIELC ObeliscN column (250 mm) with 50% mobile phase A, 50% mobile phase B for 30 minutes. Mobile phase A: 0.1% formic acid; mobile phase b: methanol with 0.1% acetic acid. Mass spectrophotometric detection was conducted using API-ES mode with an Agilent 6120 Quadroploe LCMS. Glycerol-3-phosphate was quantified by selected ion monitoring of ion 171, DHAP quantified by selected ion monitoring of ion 169, the three aldol condensation products fructose-1,6-biphosphate, “AP” and “XP” were quantified by selected ion monitoring of GCMS analysis of glycerol, glycerol-3-phosphate (G3P) and DHAP.

All three analytes can be separated and detected after derivatisation with MSTFA in pyridine. Samples were snap frozen in liquid nitrogen and then freeze-dried overnight. The resultant freeze-dried powder was resuspended in 50 μL 240 mM methoxyamine-HCl in pyridine. After incubation at 65° C. for 50 minutes, 80 μL of MSTFA was added and the samples incubated at 65° C. for a further 50 minutes. Centrifuge at 10,000 g for 10 mins. Samples can be stored at −20° C. for up to 5 days. GC-MS separation was performed with HP5-MS column (Agilent) using the following program. 1 mL/min He at 100° C., hold for 0.2 min then ramp at 10° C./min to 250° C. and hold for 10 min. Injector temperature: 280° C. 1 μL sample was injected and after 4 mM, products were detected by selected ion monitoring for DHAP (m/z 400, 315, 299, 73), G3P (m/z 357, 299, 73) and glycerol (m/z 205, 147, 73).

Peak area range disparity makes this method most useful for glycerol and glycerol-3-phosphate, and not useful for DHAP at concentrations less than 100 μM.

### Synthesis of N⁶-2AE-NAD

To a solution of NAD (1 g, 1.505 mmol) dissolved in 2 mL deionised water was added dropwise ethyleneimine (4.25 mmol) with the solution maintained at a pH of 3.2 with the addition of 70% perchloric acid. The reaction mixture was stirred at room temperature for 50 h with the pH maintained from 2-3, before the addition of 1.75 mL deionised water to solubilise precipitate. The product was precipitated by the addition of ice-cold ethanol and the precipitate washed with ethanol. The resulting mix of N1-2AE-NAD and NAD was dissolved in water (10 mL) and adjusted to pH 6.5 with 0.1 M LiOH. The solution was stirred at 50° C. for 7 h with the pH maintained at 6.5 before being lyophilised To yield the product, as a mixture of N⁶-2AE-NAD and NAD.

### Synthesis of NAD-2AE-PEG₂₄-MAL

To a stirred solution of N⁶-2AE-NAD/NAD (14.7 mg mix, approximately 0.0104 mmol N⁶-2AE-NAD) in PBS (pH 7.4, 1.0 mL) was added a solution of Mal-PEG₂₄-NHS (17.4 mg, 0.0124 mmol) in PBS (1 mL). The solution was stirred at R/T, O/N. The mixture was analysed by HPLC (0→50% MeCN+0.1% TFA over 18 mins). Rt 17.8 mins ESI+ found 662.62 (M/3, calcd 662.65) and 993.42 (M/2, calcd 993.98). The mixture was purified by pHPLC and fractions at Rt 17.8 mins combined and lyophilised to yield pure NAD-2AE-PEG₂₄-MAL (5.4 mg, 26%).

### Conjugation of NAD-2AE-PEG₂₄-MAL to BiF2

The NTA-purified BiF2 was further purified by gel filtration on a Superdex S200 2660 column equilibrated with PBS containing 0.1 mM TCEP. The major peak eluting at 177 mL (the expected volume for dimeric BiF2) was collected and desalted into degassed PBS. The protein was collected and to the BiF2 solution (60 mL, 7.8 μM) was added 0.58 mL 0.8 mM NAD-2AE-PEG₂₄-MAL (equimolar amounts). The reaction proceeded at 4° C. for 1 h before the addition of TCEP to a final concentration of 1 mM. The protein conjugate was purified by gel filtration in PBS containing 0.1 mM TCEP as described above with monitoring of the absorbance at 259, 280 and 450 nm. The main peak of protein eluting at 177 mL was collected and concentrated (Amicon 10 kDa MWCO concentrator). The protein was analysed by SDS-PAGE on an Invitrogen 4-12% gradient gel under reducing conditions. The UV-vis spectrum of the protein was determined on a Varian Cary Bio 50 Spectrophotometer. To 0.5 mL of protein was added 1 mL 7 M GuHCl and the mixture incubated for 30 min at room temperature before being concentrated through a Pall Nanosep 10 kDa MWCO concentrator. The retentate (100 μl) was removed and the membrane washed 2×0.5 mL 7 M GuHCl then 0.5 mL PBS containing 0.1 mM TCEP. The washings were combined with the retentate and the UV-vis spectrum of retentates and filtrates recorded.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from AU 2015902880 filed 20 Jul. 2015 and 2015902961 filed 24 Jul. 2015, the disclosures of which are incorporated herein by reference.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Albayrak et al. (2002) Enzyme and Microbial Technology 31:371-383.
Aplin and Wriston (1981) CRC Crit. Rev. Biochem. 10:259-306.
Ausubel et al. (editors) (1988), Current Protocols in Molecular Biology, Greene Pub. Associates and Wiley-Interscience, including all updates until present.
Brown (editor) (1991) Essential Molecular Biology: A Practical Approach, Volumes 1 and 2, IRL Press.
Buckmann et al. (1989) Adv. Biochem. Engin./Biotech. 69:97-152.
Buckman and Wray (1992) Biotechnol. Appl. Biochem. 15:303-310.
Bueckmann (1993) Eur. J. Biochem. 213:947-56.
Bueckmann (1996) Eur. J. Biochem. 238:519.
Bueckmann (2002) JACS 124:6487.
Butt (2000) Reaction Kinetics and Reactor Design, CRC Press 2nd Ed., p. 332.
Copeland et al. (1995) Bioorg. Med. Chem. Lett. 17:1947-1952.
Coligan et al. (editors) Current Protocols in Immunology, John Wiley & Sons (including all updates until present).
Coligan et al. (editors) (2013) Current Protocols in Protein Science, John Wiley & Sons (including all updates until present).
Cybulski and Moulijn (2005) Structured Catalysts and Reactors, Taylor & Francis 2nd Ed., p. 51.
Damborsky and Brezovsky (2014) Current Opinion in Chemical Biology 19(8):8-16.
Edwards et al. (2011) Cellulose 18:1239-1249.
Fuller and Bright (1980) Eur. J. Biochem. 103:421.
Glover and Hames (editors) (1995 and 1996) DNA Cloning: A Practical Approach,
Volumes 1-4, IRL Press.
Gundersen et al. (2014) Appl Microbiol Biotechnol. 98(1):219-230.
Harayama (1998) Trends Biotech. 16:76-82.
Harlow and Lane (editors) (1988) Antibodies: A Laboratory Manual, Cold Spring Harbour Laboratory.
Hessel et al. (2015) Novel Process Windows: Innovative Gates to Intensified and Sustainable Chemical Processes, Wiley VCH, 248.
Hermanson (2013) Bioconjugate Techniques, Third Edition Elsevier.
Hettwer et al. (2002) Catalysis B Enzymic 19-20:215-222.
Huang et al. (2007) Protein Expression and Purification 54:94-100.
Kim et al. (2007) Enzyme and Microbial Technology 40:1782-1787.
Kolb et al. (2001) Angew Chem Int Ed Engl. 40:2004-2021.
Lee et al. (2006) Biophys. Res. Comm. 347:616-625.
Leonida et al. (2001) Curr. Med. Chem. 8:345-369.
Li et al. (2009) Tetrahedron 65:7935-7941.
Malkoch et al. (2005) J. Am. Chem. Soc. 127:14942-14949.
Manco et al. (1998) Biochem J. 332 (Pt 1):203-12.
Mazid et al. (1993) Biotechnology 11:690-695.
Mosbach (1991) Biotechnology 9:280.
Murphy (2004) Analytical Biochemistry, 327:61-7.
Perbal (1984) Practical Guide to Molecular Cloning, John Wiley and Sons.
Pettigrew (2009) Arch. Biochem Biophys. 492:29-39.
Prieto-Blanc et al. (2010) Talanta 80:2083-2092.
Roberts et al. (2002) Advanced Drug Delivery Reviews 54:459-476.
Rocha-Martin et al. (2012) ChemCatChem. 4:1279-1288.
Sambrook et al. (1989) Molecular Cloning: A Laboratory Manual, Cold Spring
Harbour Laboratory Press.
Sakasegawa et al. (2004) Protein Science 13:3161-3171.
Sauve (2011) Org. & Biomol. Chem. 9:987.
Veronese et al. (1985) Applied Biochem. and Biotech. 11:141-152.
Wang et al. (2004) Biotech. And Bioeng. 87:178.
Williams and Morrison (1979) Methods Ezymol. 63:437-467.
Willner et al. (2002) JACS. 124:14724
Willner et al. (2009) JACS. 131:5028.
Willner et al. (2009) Nature Nanotech. 4:249.
Witke and Gotz (1993) J. Bacteriol. 175:7495-7499.
Zalipsky (1995) Bioconjugate Chem. 6:150-165.
Zhao et al. (2003) Curr Opin Biotechnol. 14:421-426.

1. An isolated enzyme complex comprising;
a) a cofactor,
b) a first enzyme that requires the cofactor to perform an enzymatic reaction, and
c) a second enzyme that recycles the cofactor,
wherein the first enzyme, second enzyme and cofactor form the enzyme complex through covalent attachments, and wherein the cofactor is covalently attached via a tether that allows the cofactor to be used by the first enzyme and recycled by the second enzyme.

2. The enzyme complex of claim 1, wherein the cofactor is selected from the group consisting of ATP/ADP, NAD+/NADH, NADP+/NADPH, and FAD+/FADH₂.

3. The enzyme complex of claim 1 or claim 2, wherein the cofactor has a ribonucleotide core.

4. The enzyme complex of claim 2 or claim 3, wherein the tether is covalently attached to the ribonucleotide core via a C—N bond to the base portion of the ribonucleotide core.

5. The enzyme complex according to any one of claims 1 to 3, wherein the tether comprises a polyethylene glycol (PEG) chain, hydrocarbon chain, a polypeptide, polynucleotide.

6. The enzyme complex of claim 5, wherein the length of the polyethylene glycol chain is PEG₂-PEG₄₈ (i.e. (—CH₂CH₂O—)₂ to (—CH₂CH₂O—)₄₈).

7. The enzyme complex of claim 5, wherein the length of the hydrocarbon chain is C₁₂-C₁₈.

8. The enzyme complex according to any one of claims 1 to 7, wherein the cofactor is tethered to one of the enzymes.

9. The enzyme complex according to any one of claims 1 to 8, wherein the first and second enzymes are covalently attached by a linker.

10. The enzyme complex of claim 9, wherein the cofactor is tethered to the linker.

11. The enzyme complex of claim 9 or claim 10, wherein the linker is an amino acid linker.

12. The enzyme complex of claim 11, wherein the linker comprises a Cys, a Thr, a Glu or a Lys amino acid residue.

13. The enzyme complex of claim 11 or claim 12, wherein the linker comprises GlySerSer amino acid residue repeats (GlySerSer)ₙ.

14. The enzyme complex of claim 13, wherein the linker comprises (GlySerSer)₃Cys(GlySerSer)₃.

15. The enzyme complex according to any one of claims 1 to 14, wherein the first enzyme is selected from the group consisting of:
i) a kinase;
ii) a dehydrogenase;
iii) an oxygenase;
iv) an aldolase;
v) a reductase;
vi) a synthase.

16. The enzyme complex according to any one of claims 1 to 15, wherein the second enzyme is selected from the group consisting of:
i) a kinase;
ii) a dehydrogenase;
iii) an oxidase;
iv) a reductase;
v) a peroxidase.

17. The enzyme complex according to any one of claims 1 to 16, wherein the complex comprises:
i) Thermococcus kodakarensis glycerol kinase, Mycobacterium smegmatis ATP kinase, ATP/ADP;
ii) Escherichia coli glycerol-3-phosphate dehydrogenase, Clostridium aminoverlaricum NADH oxidase, NAD/NADH;
iii) Shewanella yellow enzyme, Geobacillus thermodenitrificans alcohol dehydrogenase, NAD/NADH;
iv) Geobacillus thermodenitrificans alcohol dehydrogenase, C. boidinii formate dehydrogenase, NADP/NADPH; or
v) Bacillus subtilis yellow enzyme, C. boidinii formate dehydrogenase, NADP/NADPH.

18. The enzyme complex according to any one of claims 1 to 17 further comprising a covalently attached conjugation module for conjugating the complex to a solid support.

19. The enzyme complex of claim 18, wherein the conjugation module is covalently attached to the first enzyme or the second enzyme by a linker.

20. The enzyme complex of claim 18 or claim 19, wherein the conjugation module is a protein.

21. The enzyme complex of claim 20, wherein the protein is selected from the group consisting of:
i) an esterase;
ii) streptavidin;
iii) glutathione S-transferase;
iv) a metal binding protein;
v) a cellulose binding protein;
vi) a maltose binding protein; and
vii) an antibody or antigen binding fragment thereof.

22. The enzyme complex of claim 21 or claim 22, wherein the linker is a linker as defined in any one of claims 11 to 14.

23. The enzyme complex according to any one of claims 18 to 22, wherein the complex comprises:
i) Thermococcus kodakarensis glycerol kinase, Mycobacterium smegmatis ATP kinase, ATP/ADP, Alicyclobacillus acidophilus esterase; or
ii) Escherichia coli glycerol-3-phosphate dehydrogenase, Clostridium aminoverlaricum NADH oxidase, NAD/NADH, Alicyclobacillus acidophilus esterase.

24. The enzyme complex according to any one of claims 18 to 23 which is covalently or non-covalently attached to the solid support.

25. The enzyme complex of claim 24, wherein the solid support is a functionalised polymer.

26. The enzyme complex of claim 25, wherein the functionalised polymer is selected from the group consisting of: agarose, cotton, polyacrylonitrile, polyester, polyamide, protein, nucleic acids, polysaccharides, carbon fibre, graphene, glass, silica and polyurethane.

27. The enzyme complex according to any one of claims 24 to 26, wherein the solid support is in the form of a bead, a matrix, a woven fibre or a gel.

28. A method for producing the enzyme complex according to any one of claims 1 to 17, the method comprising:
i) expressing a polynucleotide encoding a chimeric protein comprising the first enzyme and the second enzyme in a host cell or cell-free expression system; and
ii) attaching the cofactor to the chimeric protein via the tether.

29. The method of claim 28, wherein the first enzyme and the second enzyme are separated by a linker and step ii) comprises covalently attaching the tether to the linker.

30. The method of claim 28 or claim 29, wherein the chimeric protein further comprises the conjugation module protein of claim 20 or claim 21.

31. The method of claim 30 which further comprises conjugating the enzyme complex to a solid support.

32. The method according to any one of claims 28 to 32, wherein the host cell is a bacterial cell, a yeast cell, a plant cell or an animal cell.

33. A method for producing a product, the method comprising,
i) providing an enzyme complex according to any one of claims 1 to 27 and a substrate of the first enzyme, and
ii) incubating the enzyme complex and substrate for a time and under conditions sufficient for the first enzyme to convert the substrate to the product and for the second enzyme to recycle the cofactor for use by the first enzyme.

34. The method of claim 33 which comprises two or more enzymatic steps and at least two of the enzymatic steps are performed using two different enzyme complexes according to any one of claims 1 to 27.

35. The method of claim 33 or claim 34 which is performed in a bioreactor.

36. The method of claim 35, wherein the bioreactor is a continuous flow bioreactor.

37. A bioreactor comprising at least one enzyme complex according to any one of claims 1 to 27.

38. A composition comprising at least one enzyme complex according to any one of claims 1 to 27.
